SEQUENCE LISTING (8 sequences)

SEQ ID NO: 1 - DNA, 22 bp, synthetic construct
  source 1..22: mol_type = other DNA
cccgaaacgccgaatataatcc

SEQ ID NO: 2 - DNA, 21 bp, synthetic construct
  source 1..21: mol_type = other DNA
ctcctgtgcacaccattttcc

SEQ ID NO: 3 - DNA, 23 bp, synthetic construct
  source 1..23: mol_type = other DNA
ccacgaaccacggcactgatttt

SEQ ID NO: 4 - DNA, 2520 bp, Homo sapiens
  source 1..2520: mol_type = other DNA
atagaccagaagaatgaatttcttacggctacctcttggctcctgtgcacaccattttcccagaactgaaaatcagtgccgtggttcgtggctctcttatcctcatgattaccgcagcaaaccgctacaatggtcagagagaatcattagtgaggtcatgccaaactgctcaatgttgttaggcgaaccacccattgacaaaaaaatcacaactggtaggaaggacatagtacatctagttatcaaaatatttgttctgtatttctgtatatctgtaaaggatttgcatacaccacatgtaacaaatattcctttgcataaggagcagaaaataatcaattgtgactaagttataccaaatctgtatgttctttcacatgagcattccatcaagagaaatgtttgcaaaatttggttatgttctcaataaatacaagtgcttctgtttctactttatatttgctgcttatcatgactatagcttctctgatacattttgttttattaatatgaattttgaggcaaataaaccaataaaccttttctaccttaatggaattaataaatcagattacaagcctatcaatacgtaatgaagtaaaatcatgctttcaattggtctttagaaacaatattaatgtattaaacacataaatgcaaattaggaaaaaaacacagttttaacttttattttctgctttttatgcttgaaacatgttaattctttcagaaaattttcttgctaaggtggtaactcaaggggaaaatatttcaactgtgggttgaattaatggcctcagttattaggtacatgataaaaagcaaataattactttgaaacaacttaaatattttaatacatttaattatacaataaaaacctacatgtttctaattaagacgactctttgacaaactagattgctttaacaattttatttttttattttattaatttttttttttagagacaagagtctcactttgttgcccagactggagtgcagtggtgcaatcatggctcactgcagcctcgaattcctgagctcaagagatccttctgtcttggcctcccaagtagctgggattataggtgtgtgccaccaaccccagataattttttaaattttctgatgaaacaaggccttgctatgttgcccaggctcgtcttgaactgttggcctcaagtgatcctcccaccttggcctcccaaagtgctggaattacaggtctgagtcaccatgcctggcttttaacaattttagtttaaaacctgctctatgtcttccctgtgattttatcaacatctaaaaaagctagcagtatatagtagtttatatacgtcccaggaaaagcctagctattttgaatttccagaatttcctctactatttttattggttaaatatcacttaagaacgtaaagtaataaaattatgtattcaactaaatgtgattataatagatgaaagattaattcttagaatgctacatgtcatacagactcattatgagtggttaaaaggtgattttcagacccttagctgtctttcagatctcaggcattatgtttcagggattttaaaatgcaccctaagtccattacttaacatttctctcaacccagctaatgtttatccaaattactttgcctactatagctggctaacatcatccctatacatatacaaaattcttcacatgatcccaatacaatacatatataacttagcttattttatagactgctgtactcccagggattcttatatcacagacactcaataaaagatacaaaacaatgcacattttactttagatcttcattttattggtgtcaaggaataagcataacacaaactctttgatatacagattttttttttgaggcggagtctcgctctgttgccaggctggaatgcaatggcgcaatctccgctcactgcaacctctgcctcccaggatcaagtgattctcctgcctcagcctcccaagtagccgggattacaggcgcatgccaccactcccggcaaatttttgtatttttagtagagacggggtttcactatgttggccaggatggtcttgaactcctgacctcagatgatccgcccacctcggcctcccaaagtgctgggattacacgtgtgagccaccaagcccggccaattttttttttttttttaagagacagggtcttgccatgttgcctgggctggtggtgaactcctaggttcaagaaattctccttcctcagcctcccaaagtgctgggattacaggcattagccactgtgcctggcctgatctacagacatttgttttatagcgtgaaatacattaaaatatagagacacggcagaggagacaaaaaagaaaaaaaaaagaaatacattaaaatagatctaaccttgggattatattcggcgtttcgggcacgaagtgcaatggtctttaggtcaagtttacaaccaagattcactgtggatacaatatttctgtaagagatttaaaaagaaaa

SEQ ID NO: 5 - DNA, 2445 bp, Homo sapiens
  source 1..2445: mol_type = other DNA
gtcgacgggcccgtttaaacttactctgaaagaattacatatttctttctttttggttgagcttttgttatttaaatacatttgatgagaggatattgaaataattaaatagcactgagctttaaattatttacaatcccctaatggaaattttcactaatgagatatcataatgaatgtgaattttatttctgaaatctctaataaatcagtcttctccctggttttcccagctcagcgcccattacgtttctgttctctttcccttagtggcattatttgtatcactgtgcatgtgcgctgccaacctgctgcccgtgcagaaactctagggtaagagctggctcctggagtcccacccaggctgcgtgtccctcacagtctgctctgtgtctatgtgtgtgtgttcctaggtacgtaatgcatggccaaagcaaggtagtgatcaaagagttcaaaatttcaatttgccccgtctgtgtatacagaagttctttccaaaaaagaaatgctttatctttgacttacctgctcaccaaaaaaagcttgcccaacttgaaacactgcctgatgatgagctagagcctgaatttgtgcaacaagtgacagaattctgttcctacatctttagccattctatgaccaagactcttccaggtggcatcatggtcaatggatctcgtctaaagaacctggtgctgacctatgtcaatgccatcagcagtggggatctgccttgcatagagaatgcagtcctggccttggctcagagagagaactcagctgcagtgcaaaaggccattgcccactatgaccagcaaatgggccagaaagtgcagctgcccatggaaaccctccaggagctgctggacctgcacaggaccagtgagagggaggccattgaagtcttcatgaaaaactctttcaaggatgtagaccaaagtttccagaaagaattggagactctactagatgcaaaacagaatgacatttgtaaacggaacctggaagcatcctcggattattgctcggctttacttaaggatatttttggtcctctagaagaagcagtgaagcagggaatttattctaagccaggaggccataatctcttcattcagaaaacagaagaactgaaggcaaagtactatcgggagcctcggaaaggaatacaggctgaagaagttctgcagaaatatttaaagtccaaggagtctgtgagtcatgcaatattacagactgaccaggctctcacagagacggaaaaaaagaagaaagaggcacaagtgaaagcagaagctgaaaaggctgaagcgcaaaggttggcggcgattcaaaggcagaacgagcaaatgatgcaggagagggagagactccatcaggaacaagtgagacaaatggagatagccaaacaaaattggctggcagagcaacagaaaatgcaggaacaacagatgcaggaacaggctgcacagctcagcacaacattccaagctcaaaatagaagccttctcagtgagctccagcacgcccagaggactgttaataacgatgatccatgtgttttactctaaagtgctaaatatgggagtttcctttttttactctttgtcactgatgacacaacagaaaagaaactgtagaccttgggacaatcaacatttaaataaactttataattattttttcaaactttcatatagagttataagattatgatgctggtatctggtaaaatgtacatcccagtagtccaatagtttaaatgtttattgcttcctttaagagattataaattgtataagggacattgtatcactgccttcatttatgcgtgatattgggatggtttcatcaggagatgctttccttgcatctcaatgtcatctgtctaatttctcataaggggattatgttacctagagcagggcttcccaaccctcaggccatagactagctctgatctgtggcctcttaggaacccggccacacagcaggaggtgagcagcaggtagcggccgcttaattaagggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagatggctcgttcggaacaccacctggatacggctgcgctgcagaccgggcagaggagcagcgccggcaccaagatgggctgccctacattgatgactcgccctcctcatcgccccacctcagcagcaagggcaggggcagccgggatgcgctggtctcgggagccctggagtccactaaagcgagtgagctggacttggaaaagggcttcacgtgacgcgtggatcc

SEQ ID NO: 6 - DNA, 1081 bp, Homo sapiens
  source 1..1081: mol_type = other DNA
gcaacaagtgacagaattctgttcctacatctttagccattctatgaccaagactcttccaggtggcatcatggtcaatggatctcgtctaaagaacctggtgctgacctatgtcaatgccatcagcagtggggatctgccttgcatagagaatgcagtcctggccttggctcagagagagaactcagctgcagtgcaaaaggccattgcccactatgaccagcaaatgggccagaaagtgcagctgcccatggaaaccctccaggagctgctggacctgcacaggaccagtgagagggaggccattgaagtcttcatgaaaaactctttcaaggatgtagaccaaagtttccagaaagaattggagactctactagatgcaaaacagaatgacatttgtaaacggaacctggaagcatcctcggattattgctcggctttacttaaggatatttttggtcctctagaagaagcagtgaagcagggaatttattctaagccaggaggccataatctcttcattcagaaaacagaagaactgaaggcaaagtactatcgggagcctcggaaaggaatacaggctgaagaagttctgcagaaatatttaaagtccaaggagtctgtgagtcatgcaatattacagactgaccaggctctcacagagacggaaaaaaagaagaaagaggcacaagtgaaagcagaagctgaaaaggctgaagcgcaaaggttggcggcgattcaaaggcagaacgagcaaatgatgcaggagagggagagactccatcaggaacaagtgagacaaatggagatagccaaacaaaattggctggcagagcaacagaaaatgcaggaacaacagatgcaggaacaggctgcacagctcagcacaacattccaagctcaaaatagaagccttctcagtgagctccagcacgcccagaggactgttaataacgatgatccatgtgttttactctaaagtgctaaatatgggagtttcctttttttactctttgtcactgatgacacaacagaaaagaaactgtagaccttgggacaatcaacatttaaataaacttta

SEQ ID NO: 7 - DNA, 2277 bp, Homo sapiens
  source 1..2277: mol_type = other DNA
gtcgaccacgtgtctagtgtgataacattttgttatcttattcattgtcttcatccctgaaatacactctgctctctcctatctctgctctgaaaggcagaaagagggcagccctctccaaggcaaaatggggctcctgtggggaacagaggggtgcctctgtcaacaaaggtgatgccacatccctttcaaccatgctgacacctctggtttttgtaaaggtgcccactcctgtgcacggtgatgcataggcacctgcaccccgtgcctaaaccctgcagctggcacgggccctgtctgctcttcattcaccgttctcacgagttgcaataagttcagccctccatgtcacactgtgttttcctaggaccggtaatgccaacttctacaaggactccggcatcacatacctgggcatcaaggccaacgacacacaggagttcaacctcagcgcttactttgaaagggctgccgacttcattgaccaggctttggctcaaaagaatggccgggtgctcgtccactgccgggaaggttatagccgctccccaacgctagttatcgcctacctcatgatgcggcagaagatggacgtcaagtctgccctgagcatcgtgaggcagaaccgtgagatcggccccaacgatggcttcctggcccagctctgccagctcaatgacagactagccaaggatttgaagttgaaaccctagttcatttcaaccaaatctgctcgagatttccgtaagttaggccgttagcaaaggtgtcaagagctgccatgtttaggaaacacactgtatactgctcccagcatcacaaggcacttgtctacaagtgtgtcccaacacagtcctgggccactttaaccaccctggggagcacataaagaagcttgccaattaggatgtccttgctctcggattattgctcggctttacttaaggatatttttggtcctaaagaagaagcagtgaagcagggaatttattctaagccaggaggccataatctcttcattcagaaaacagaagaactgaaggcaaagtactatcgggagcctcggaaaggaatacaggctgaagaagttctgcagaaatatttaaagtccaaggagtctgtgagtcatgcaatattacagactgaccaggctctcacagagacggaaaaaaagaagaaagaggcacaagtgaaagcagaagctgaaaaggctgaagcgcaaaggttggcggcgattcaaaggcagaacgagcaaatgatgcaggagagggagagactccatcaggaacaagtgagacaaatggagatagccaaacaaaattggctggcagagcaacagaaaatgcaggaacaacagatgcaggaacaggctgcacagctcagcacaacattccaagctcaaaatagaagaacggtaacagcgtgtttacagagaatctaactcgatgatgaatacaactacgccattaccaaagcaaacgtctgtgactgaagtttcggtgttaaaagaccacgatcctccttgacgagttttgtttttcaaaatggtgcaataatttaagtggcatcttctctcccaccgggtctagcttcggagagttctgggattgtaccgcagctgcaaaatattgtatccacagtgaatcttggttgtaaacttgacctaaagaccattgcacttcgtgcccgaaacgccgaatataatcccaagcggtttgctgcggtaatcatgaggataagagagccacgaaccacggcactgattttcagttctgggaaaatggtgtgcacaggagccaagagtgaagaacagtccagactggcagcaagaaaatatgctagagttgtacagaagttgggttttccagctaagttcttggacttcaagattcagaatatggtggggagctgtgatgtgaagtttgctagcgcggccgctgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggagtttaaaccatatgggatcc

SEQ ID NO: 8 - DNA, 1480 bp, Homo sapiens
  source 1..1480: mol_type = other DNA
cctgggcatcaaggccaacgacacacaggagttcaacctcagcgcttactttgaaagggctgccgacttcattgaccaggctttggctcaaaagaatggccgggtgctcgtccactgccgggaaggttatagccgctccccaacgctagttatcgcctacctcatgatgcggcagaagatggacgtcaagtctgccctgagcatcgtgaggcagaaccgtgagatcggccccaacgatggcttcctggcccagctctgccagctcaatgacagactagccaaggatttgaagttgaaaccctagttcatttcaaccaaatctgctcgagatttccgtaagttaggccgttagcaaaggtgtcaagagctgccatgtttaggaaacacactgtatactgctcccagcatcacaaggcacttgtctacaagtgtgtcccaacacagtcctgggccactttaaccaccctggggagcacataaagaagcttgccaattaggatgtccttgctctcggattattgctcggctttacttaaggatatttttggtcctaaagaagaagcagtgaagcagggaatttattctaagccaggaggccataatctcttcattcagaaaacagaagaactgaaggcaaagtactatcgggagcctcggaaaggaatacaggctgaagaagttctgcagaaatatttaaagtccaaggagtctgtgagtcatgcaatattacagactgaccaggctctcacagagacggaaaaaaagaagaaagaggcacaagtgaaagcagaagctgaaaaggctgaagcgcaaaggttggcggcgattcaaaggcagaacgagcaaatgatgcaggagagggagagactccatcaggaacaagtgagacaaatggagatagccaaacaaaattggctggcagagcaacagaaaatgcaggaacaacagatgcaggaacaggctgcacagctcagcacaacattccaagctcaaaatagaagaacggtaacagcgtgtttacagagaatctaactcgatgatgaatacaactacgccattaccaaagcaaacgtctgtgactgaagtttcggtgttaaaagaccacgatcctccttgacgagttttgtttttcaaaatggtgcaataatttaagtggcatcttctctcccaccgggtctagcttcggagagttctgggattgtaccgcagctgcaaaatattgtatccacagtgaatcttggttgtaaacttgacctaaagaccattgcacttcgtgcccgaaacgccgaatataatcccaagcggtttgctgcggtaatcatgaggataagagagccacgaaccacggcactgattttcagttctgggaaaatggtgtgcacaggagccaagagtgaagaacagtccagactggcagcaagaaaatatgctagagttgtacagaagttgggttttccagctaagttcttggacttcaaga

(19) United States
(12) Patent Application Publication
(10) Pub. No.: US 2023/0140360 A1
(43) Pub. Date: May 4, 2023

(54) COMPOSITIONS AND METHODS OF DIAGNOSING AND TREATING TUBERCULOSIS

(71) Applicant: Cepheid, Sunnyvale, CA (US)

(72) Inventor: Erik SÖDERSTEN, Solna (SE)

(21) Appl. No.: 18/049,165

(22) Filed: Oct. 24, 2022

Related U.S. Application Data
(60) Provisional application No. 63/270,720, filed on Oct. 22, 2021.

(51) Int. Cl. G01N 33/569

(52) U.S. Cl. CPC G01N 33/5695

(57) ABSTRACT

Compositions and methods for detecting Mycobacterium tuberculosis (MTB) infection in a patient suspected of being infected with MTB and for distinguishing active tuberculosis (ATB), incipient tuberculosis (ITB) or subclinical tuberculosis (STB) from latent tuberculosis and other pulmonary and infectious diseases are provided. The methods may also be used to monitor treatment responses of MTB infected patients. Changes in the expression level of genes are used to aid in the diagnosis, prognosis, and treatment of tuberculosis.

### CROSS-REFERENCE TO RELATED APPLICATIONS

This application claims priority to U.S. Provisional Application No. 63/270,720, filed Oct. 22, 2021, the disclosure of which is incorporated herein by reference.

### SEQUENCE LISTING

The Sequence Listing XML associated with this application is provided electronically in XML format and is hereby incorporated by reference into the specification. The name of the XML file containing the Sequence Listing XML is “CEPH-002_001US_SeqList”. The XML file is 17,758 bytes in size, created on Oct. 24, 2022, and is being submitted electronically via USPTO Patent Center.

### FIELD OF THE DISCLOSURE

Compositions and methods for aiding diagnosis, prognosis, and treatment of tuberculosis (TB) are provided. In particular, the disclosure relates to markers and panels of markers useful to detect patients with an active infection of Mycobacterium tuberculosis (MTB) and also distinguish active tuberculosis (ATB) from latent tuberculosis and other pulmonary and infectious diseases, for monitoring responses to anti-TB treatment and predict progression to ATB from incipient TB.

### BACKGROUND

Tuberculosis (TB) is a worldwide public health issue, with 9 million new infections and 1.5 million deaths in 2018 (Global Tuberculosis Programme, World Health Organization. Global tuberculosis report. Geneva, Switzerland: World Health Organization; 2019). Host response type polymerase chain reaction (PCR) tests that detect a patient's specific transcriptional response hold promise for detecting active tuberculosis infection. The use of gene expression data as a biomarker to improve diagnosis and prognosis of diseases is dependent on a number of factors. For example, where the expression levels of multiple target genes are combined in a defined manner to provide an expression signature or an expression score for a biomarker, accurate measurement by RT-PCR relies on consistent levels of transcript stability for each target gene. If one or more target genes behave differently relative to the other target genes, the resulting signature or score will be affected. Variation in experimental conditions caused by preanalytical factors can substantially and independently influence transcript stability of genes and hence gene expression data. This is particularly noteworthy in the clinical setting, as differences in sample collection, sample processing, and assay performance in the different clinical centers have previously been shown to influence the accuracy of the gene expression score. However, verification that transcript stability for multiple genes are adequately consistent among samples to generate a signature is generally not performed.

There is a need for diagnostic test methods and kits that utilize biomarkers that are stable and can be used in gene expression studies. Particularly, there is a need for diagnostic test methods and kits with improved accuracy and reliability for detecting tuberculosis infections. The present disclosure addresses these and other needs.

### SUMMARY

Compositions and methods for identifying in individuals the presence or absence of tuberculosis (TB) and further determining the disease stage of those individuals that are infected with TB are disclosed. The disclosed compositions and methods utilize a combination of biomarkers that exhibit low (or similar) variability in expression levels over time and various sample conditions. The compositions and methods are particularly useful in locations where variability in sample collection is high or where there is a need for transport and/or storage of the sample. Particularly disclosed herein are biomarkers for detecting active tuberculosis (ATB), incipient tuberculosis (ITB), subclinical tuberculosis (STB), latent tuberculosis (LTB) or negative for TB; distinguishing ATB from LTB and other pulmonary and infectious diseases; for monitoring responses to tuberculosis treatment; predicting progression to ATB from ITB; and predicting low risk or high risk of developing ATB. Methods for treating a patient identified as having ATB, identified as having ITB, identified as having LTB, identified as being at risk of progression or developing ATB, or who are being monitored for treatment using the methods described herein are also disclosed. The combination of biomarkers herein have surprisingly similar transcript stability at room temperature, at elevated temperatures (such as 45° C. or greater, 40° C. or greater, 35° C. or greater, 30° C. or greater, or 27° C. or greater), or at lower temperatures (such as 23° C. or less, 20° C. or less, 15° C. or less, 10° C. or less, or 5° C. or less). The biomarkers also exhibit surprisingly similar transcript stability at the aforesaid temperatures over time (such as over a period of 1 hr or greater, up to 2 hrs or greater, up to 5 hrs or greater, up to 8 hrs or greater, up to 12 hrs or greater, up to 18 hrs or greater, or up to 24 hrs or greater).

In certain aspects, the disclosure provides methods for treating a patient for tuberculosis, comprising: (a) identifying the patient as having active tuberculosis based on the expression levels of the biomarkers GBP5, DUSP3, and TBP in a biological sample; and (b) administering an effective amount of at least one antibiotic to the patient.

In certain other aspects, the disclosure provides methods for diagnosing and treating different stages of tuberculosis infection in a patient comprising: (a) obtaining a first biological sample from the patient; (b) measuring levels of expression of the biomarkers DUSP3, GBP5, and TBP in the first biological sample; (c) comparing the level of expression of each of the biomarkers to a reference value for that biomarker or to a control; (d) diagnosing the patient as having active tuberculosis or incipient tuberculosis by analyzing the expression levels of each biomarker in conjunction with respective reference value ranges for each biomarker; and (e) administering an effective amount of at least one antibiotic to the patient.

In certain other aspects, the disclosure provides a method of diagnosing different stages of tuberculosis infection in a patient comprising: (a) measuring levels of expression of the biomarkers DUSP3, GBP5, and TBP in a biological sample obtained from the subject; (c) comparing the level of expression of each of the biomarkers to a reference value for that biomarker or to a control; (d) diagnosing the patient as having active tuberculosis or incipient tuberculosis by analyzing the expression levels of each biomarker in conjunction with respective reference value ranges for each biomarker.

In certain other aspects, the disclosure provides a method of treating different stages of tuberculosis infection in a patient comprising: (a) measuring levels of expression of the biomarkers DUSP3, GBP5, and TBP in a biological sample obtained from the subject; (c) comparing the level of expression of each of the biomarkers to a reference value for that biomarker or to a control; (d) diagnosing the patient as having active tuberculosis or incipient tuberculosis by analyzing the expression levels of each biomarker in conjunction with respective reference value ranges for each biomarker; and (e) administering an effective amount of at least one antibiotic to the patient.

In further aspects, the disclosure provides methods for monitoring a tuberculosis infection in a subject, particularly after tuberculosis treatment comprising: (a) measuring levels of expression of the biomarkers DUSP3, GBP5, and TBP in a first biological sample obtained from the patient; (b) measuring levels of expression of the biomarkers GBP5, DUSP3, and TBP in a second biological sample from the patient, wherein the second biological sample is obtained from the patient at a second time point after tuberculosis treatment; (c) comparing the levels of expression of the biomarkers in the first biological sample to the levels of expression of the biomarkers in the second biological sample, or calculating TB scores based on the levels of expression of the biomarkers in the first biological sample and the second biological sample, to determine if the tuberculosis infection in the patient is improving or worsening; and (d) optionally administering a second treatment regimen that results in improved health conditions of the patient.

The patient described herein may be (i) suspected of being infected with TB, (ii) suspected of having ATB, incipient TB or subclinical TB, (iii) at risk of having ATB (for example HIV co-infected, household contacts of patient with ATB), (iv) actively being treated for ATB and being tested to monitor treatment response or (v) being treated with TPT and being tested to monitor treatment response. As described herein, conventional TB tests require coughing up mucus from the lower respiratory tract (sputum sample), which can be unsafe to collect and handle for healthcare workers. Sputum is also difficult and invasive to collect from children and is not produced by many HIV+patients. In some examples, the patient described herein can be one that is suspected of not producing sputum or of being difficult to collect a sufficient sputum sample from, for example, children, HIV+, and/or has extrapulmonary TB.

The biological sample collected from the patient can be whole blood, sputum, saliva, nasal swab, peripheral blood mononuclear cells (PBMCs), monocytes, or macrophages. Preferably, the sample is not a sputum sample. In some instances, the biological sample is whole blood collected from the patient by capillary (e.g., from a finger-stick) or venous blood draw. When the sample is whole blood, the blood does not require processing/spinning but can be supplemented with anticoagulant agents (e.g., EDTA) or RNA-stabilization buffers. Prior to analysis of the selected biomarkers (DUSP3, GBP5, and TBP) such as by PCR, the biological sample can be transported and/or stored at room temperature, at elevated temperatures (such as 45° C. or greater, 40° C. or greater, 35° C. or greater, 30° C. or greater, or 27° C. or greater), or lower temperatures (such as 23° C. or less, 20° C. or less, 15° C. or less, 10° C. or less, or 5° C. or less), and still provide accurate and reliable gene expression data. In some cases, the biological sample can be maintained at said temperatures for a period of 1 hr or greater, 2 hrs or greater, 5 hrs or greater, 8 hrs or greater, 12 hrs or greater, 18 hrs or greater, or 24 hrs or greater. Accordingly, the target combination of biomarkers (DUSP3, GBP5, and TBP) enables longer hold time of biological samples in room temperature or elevated temperatures compared to other known combinations of biomarkers for use in the methods herein.

The biomarkers DUSP3 and GBP5 show changes in expression in response to active tuberculosis infection, and importantly relative to the infection stage. The change in expression may be overexpression or under expression and may vary from gene to gene. In certain embodiments, GBP5 and DUSP3 are overexpressed in patients who have a tuberculosis infection. In certain embodiments, GBP5 and DUSP3 are overexpressed in patients who have an active tuberculosis infection. In certain embodiments, GBP5 and DUSP3 are overexpressed in patients who have an incipient tuberculosis infection. In certain embodiments, GBP5 and DUSP3 are overexpressed in patients who have a subclinical tuberculosis infection. TBP may be under-expressed or have a constant level of expression in patients who have active or incipient tuberculosis infection. In certain embodiments, TBP exhibits a constitutive expression throughout different conditions of temperature and over time and its expression level is similar relative to the expression levels of DUSP3 and GBP5. Particularly, the expression stability of TBP relative to DUSP3 and GBP5 is analogous at low temperatures, room temperature, or elevated temperatures over time, and therefore a resulting expression score from the 3-gene equation exemplified will not be affected.

The expression levels of the biomarkers can be measured using PCR. For example, the method can comprise quantitative PCR or real time RT-PCR wherein RNA is reverse transcribed to create cDNA and cDNA is amplified by PCR. The RT-PCR reaction takes less than 2 hours from an initial denaturation step through a final extension step. In some embodiments, the reaction takes less than 2 hours, less than 1 hour, less than 45 minutes, less than 40 minutes, less than 35 minutes, less than 30 minutes, or less than 25 minutes from initial denaturation through the last extension.

The method can comprise contacting nucleic acids from the sample with a primer pair for detecting each of the biomarkers. In some embodiments, the primer pair comprises a first primer and a second primer, wherein the first primer comprises a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of each biomarker, and wherein the second primer comprises a sequence that is at least 85%, at least 90%, at least 95%, or 100% complementary to at least 15, at least 16, at least 17, at least 18, at least 19, at least 20, at least 21, at least 22, at least 23, at least 24, or at least 25 contiguous nucleotides of each biomarker.

The method can comprise forming an amplicon from each primer pair when the target of the primer pair is present. In some embodiments, each primer pair produces an amplicon that is 50 to 500 nucleotides long, 50 to 400 nucleotides long, 50 to 300 nucleotides long, 50 to 200 nucleotides long, or 50 to 150 nucleotides long. The amplicons can be contacted with at least one probe. In some embodiments, the probe comprises a sequence that is at least 85%, at least 90%, at least 95%, or 100% identical or complementary to at least 13, at least 14, at least 15, at least 16, at least 17, at least 18, at least 19, at least 20, at least 21, at least 22, at least 23, at least 24, or at least 25 contiguous nucleotides of the biomarker. In some embodiments the method comprises contacting the amplicons with a probe for each biomarker to be analyzed.

Each probe can comprise a detectable label. In some embodiments, each probe comprises a fluorescent dye and a quencher molecule. In some instances, the probes comprise detectable labels that are detectably different. In other instances, the probes comprise detectable labels that are not detectably different. In some embodiments, each probe consists of 13 to 30 nucleotides.

The methods disclosed herein may comprise forming an exogenous control amplicon. In some embodiments, the method comprises contacting the exogenous control amplicon with a control probe capable of selectively hybridizing with the exogenous control amplicon.

As described above, the patient may be diagnosed as either having ATB, ITB, LTB, or not ATB, risk for progression into ATB from ITB, low risk or high risk of developing ATB, or monitored for determining efficacy of a therapy for treating tuberculosis progression to ATB. After a diagnosis is made an effective amount of at least one antibiotic may be administered to the patient if the patient is diagnosed with ATB or ITB tuberculosis. The selection of antibiotic and the duration of treatment may be selected based on the diagnosis. In some cases, the patient can be administered at least one antibiotic selected from the group consisting of rifampicin, isoniazid, pyrazinamide, ethambutol, rifapentine, ethionamide, moxifloxacin, and streptomycin. Patients with active tuberculosis can be further administered an effective amount of a corticosteroid. For patients being monitored for efficacy of tuberculosis treatment, if the tuberculosis infection in the patient is worsening, a second treatment regimen can be administered to the patient that results in improved health conditions of the patient. If the tuberculosis infection in the patient is improving, the patient can continue with their current tuberculosis treatment. In some instances, the methods described herein may be used as part of a tuberculosis triage test. A TB triage test should stratify individuals for either confirmatory TB diagnostic testing (for triage test-positive patients) or further investigation of likely non-TB aetiologies (for triage test-negative patients).

Kits are also provided herein. The kits can comprise primers and probes for detecting and/or measuring expression levels of the biomarkers GBP5, DUSP3, and TBP, wherein the primers comprise a first PCR primer pair for detecting the biomarker GBP5, a second PCR primer pair for detecting the biomarker DUSP3, and a third PCR primer pair for detecting the biomarker TBP; and wherein the probes comprise at least one PCR probe for detecting the biomarker GBP5, at least one PCR probe for detecting the biomarker DUSP3, and at least one PCR probe for detecting the biomarker TBP. Each probe can comprise a detectable label. For example, each probe comprises a fluorescent dye and a quencher molecule. In some embodiments, the probe is a fluorescence resonance energy transfer (FRET) probe.

The kit can comprise an exogenous control. In some embodiments, the exogenous control is an RNA control. In some embodiments, the RNA control is packaged in a bacteriophage protective coat (e.g., ARMORED® RNA). In some embodiments, the kit comprises dNTPs and/or a thermostable polymerase. In some embodiments, the kit comprises a reverse transcriptase. In some embodiments the kit contains primers and probes for detecting an endogenous control RNA.

The methods, compositions, and kits disclosed herein provide a blood-based, rapid point-of-care host response test for active, incipient, and subclinical tuberculosis; for distinguishing active tuberculosis (ATB) from latent tuberculosis and other pulmonary and infectious diseases; for monitoring responses to tuberculosis treatment and predict progression to ATB. The methods, compositions, and kits are suitable for linking more patients to appropriate care in both remote areas as well as for key patient populations.

### BRIEF DESCRIPTION OF THE FIGURES

FIG. 1A shows graphs of the change in stability of KLF2, DUSP3, and GBP5 biomarkers and corresponding delta TB-score at room temperature (RT) after blood sampling.

FIG. 1B shows a graph of delta TBP-score over time as calculated from stability scores of TBP, DUSP3, and GBP5 at room temperature (RT) after blood sampling.

FIG. 2A shows graphs of the change in stability of KLF2, DUSP3, and GBP5 biomarkers and corresponding delta TB-score at 35° C. after blood sampling.

FIG. 2B shows a graph of delta TBP-score over time as calculated from stability scores of TBP, DUSP3, and GBP5 at 35° C. after blood sampling.

FIG. 3A, FIG. 3B and FIG. 3C are graphs showing performance of the XPERT TB Host Response RUO prototype cartridge evaluated against Mtb culture using receiver operating characteristic (ROC) analysis for different score equations; delta TB-score=(GBP5+DUSP3)/2−KLF2, delta TBP-score=(GBP5+DUSP3)/2−TBP or delta TBPKLF2score=(GBP5+DUSP3)/2−(TBP+KLF2)/2.

FIG. 3D, FIG. 3E and FIG. 3F are graphs showing performance of the XPERT TB Host Response RUO prototype cartridge evaluated against the XPERT MTB/RIF cartridge assay using receiver operating characteristic (ROC) analysis for different score equations; delta TB-score=(GBP5+DUSP3)/2−KLF2, delta TBP-score=(GBP5+DUSP3)/2−TBP or delta TBPKLF2score=(GBP5+DUSP3)/2−(TBP+KLF2)/2.

FIG. 4A, FIG. 4B, and FIG. 4C are graphs showing changes in delta TB-score (FIG. 4A), delta TBP-score (FIG. 4B), and delta TBPKLF2-score (FIG. 4C) over time. FIG. 4D is a graph showing changes in delta TB-score and delta TBP-score during an accelerated kit stability study at different temperatures using pooled frozen donor blood. No score drift was observed after 13 months at 35° C. FIG. 4E is a graph showing changes in delta TBP-score during an accelerated kit stability study at different temperatures using pooled frozen donor blood. No score drift was observed up to 8 weeks at 35° C., up to 6 weeks at 50° C., and up to 4 weeks at 55° C.

FIG. 5 is a graph showing results from a semi-quantitative TB Fingerstick assay on GeneXpert.

FIG. 6 depicts an oligonucleotide sequence of TBP comprising exons 3 and 4 (SEQ ID NO: 4).

FIG. 7 shows a graph of delta TB-scores and TBP-scores in venous blood samples from 6 donors analyzed on a prototype cartridge at 0, 1, 3, 5 and 7 hours after draw and at 21° C., 25° C., 28° C. and 35° C. Data showing average (8 technical replicates at t=0, 6 technical replicates at other time points) delta TB-score and TBP-score per donor from t=0. Delta TBP score showed a lower variation from t=0 at all temperatures and time-points.

FIG. 8A and FIG. 8B depict oligonucleotide sequences of GBP5.

FIG. 9A and FIG. 9B depict oligonucleotide sequences of DUSP3.

### DETAILED DESCRIPTION

Unless otherwise defined, all technical and scientific terms used herein have the same meaning as commonly understood by one of ordinary skill in the art to which this disclosure belongs. In the specification, the singular forms also include the plural unless the context clearly dictates otherwise; as examples, the terms “a,” “an,” and “the” are understood to be singular or plural and the term “or” is understood to be inclusive. By way of example, “an infection” means one or more infections. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.” Unless specifically stated or obvious from context, as used herein, the term “or” is understood to be inclusive and covers both “or” and “and”.

### Definitions

To facilitate an understanding of the present disclosure, a number of terms and phrases are defined below:

As used herein, the terms “detect”, “detecting” or “detection” may describe either the general act of discovering or discerning or the specific observation of a detectably labeled composition.

As used herein, the term “detectably different” refers to a set of labels (such as dyes) that can be detected and distinguished simultaneously.

As used herein, the terms “patient” and “subject” are used interchangeably to refer to a human. In some embodiments, the methods described herein may be used on samples from non-human animals.

As used herein, the terms “latent tuberculosis”, “latent TB”, “LTB” or “LTBI” refer to an infection with viable M. tuberculosis for which progression to TB disease is not expected to occur in the near future in the absence of any significant immunological compromise. This represents a more conceptual analogue to the current WHO definition, which considers LTBI “as having evidence of TB infection and no clinical, radiological or microbiological evidence of active TB disease”.

As used herein, the terms “incipient tuberculosis” or “incipient TB” or “ITB” refer to an infection with viable M. tuberculosis bacteria that is likely to progress to active disease in the absence of further intervention but has not yet induced clinical symptoms, radiographic abnormalities, or microbiologic evidence consistent with active TB disease.

As used herein, the terms “subclinical tuberculosis” or “subclinical TB” or “STB” refer to a disease due to viable M. tuberculosis bacteria that does not cause clinical TB-related symptoms but causes other abnormalities that can be detected using existing radiologic or microbiologic assays.

As used herein, the terms “active tuberculosis” or “ATB” refer to a disease due to viable M. tuberculosis that causes clinical symptoms with radiographic abnormalities or microbiologic evidence consistent with active TB disease. This would remain consistent with the current WHO definition, which considers active TB disease as “symptomatic patients with radiological or microbiological evidence of M. tuberculosis”.

As used herein, the terms “oligonucleotide,” “polynucleotide,” “nucleic acid molecule,” and the like, refer to nucleic acid-containing molecules, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “oligonucleotide,” refers to a single-stranded polynucleotide having fewer than 500 nucleotides. In some embodiments, an oligonucleotide is 8 to 200, 8 to 100, 12 to 200, 12 to 100, 12 to 75, or 12 to 50 nucleotides long. Oligonucleotides may be referred to by their length, for example, a 24-residue oligonucleotide may be referred to as a “24-mer.”

As used herein, the term “complementary” to a target RNA (or target region thereof), and the percentage of “complementarity” of the probe sequence to that of the target RNA sequence is the percentage “identity” to the sequence of target RNA or to the reverse complement of the sequence of the target RNA. In determining the degree of “complementarity” between probes used in the compositions described herein (or regions thereof) and a target RNA, such as those disclosed herein, the degree of “complementarity” is expressed as the percentage identity between the sequence of the probe (or region thereof) and sequence of the target RNA or the reverse complement of the sequence of the target RNA that best aligns therewith. The percentage is calculated by counting the number of aligned bases that are identical as between the 2 sequences, dividing by the total number of contiguous nucleotides in the probe, and multiplying by 100. When the term “complementary” is used, the subject oligonucleotide is at least 90% complementary to the target molecule, unless indicated otherwise. In some embodiments, the subject oligonucleotide is at least 91%, at least 92%, at least 93%, at least 94%, at least 95%, at least 96%, at least 97%, at least 98%, at least 99%, or 100% complementary to the target molecule.

A “primer” or “probe” as used herein, refers to an oligonucleotide that comprises a region that is complementary to a sequence of at least 8 contiguous nucleotides of a target nucleic acid molecule, such as DNA (e.g., a target gene) or an mRNA (or a DNA reverse-transcribed from an mRNA). In some embodiments, a primer or probe comprises a region that is complementary to a sequence of at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides of a target molecule. When a primer or probe comprises a region that is “complementary to at least x contiguous nucleotides of a target molecule,” the primer or probe is at least 95% complementary to at least x contiguous nucleotides of the target molecule. In some embodiments, the primer or probe is at least 96%, at least 97%, at least 98%, at least 99%, or 100% complementary to the target molecule.

The term “primer pair” refers to a set of primers including a 5′ “upstream primer” or “forward primer” that hybridizes with the complement of the 5′ end of the DNA sequence to be amplified and a 3′ “downstream primer” or “reverse primer” that hybridizes with the 3′ end of the sequence to be amplified. As will be recognized by those of skill in the art, the terms “upstream” and “downstream” or “forward” and “reverse” are not intended to be limiting, but rather provide illustrative orientations in some embodiments.

The term “nucleic acid amplification,” encompasses any means by which at least a part of at least one target nucleic acid is reproduced, typically in a template-dependent manner, including without limitation, a broad range of techniques for amplifying nucleic acid sequences, either linearly or exponentially. Exemplary means for performing an amplifying step include polymerase chain reaction (PCR), ligase .chain reaction (LCR), ligase detection reaction (LDR), multiplex ligation-dependent probe amplification (MLPA), ligation followed by Q-replicase amplification, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), recombinase polymerase amplification and the like, including multiplex versions and combinations thereof, for example but not limited to, OLA/PCR, PCR/OLA, LDR/PCR, PCR/PCR/LDR, PCR/LDR, LCR/PCR, PCR/LCR (also known as combined chain reaction—CCR), digital amplification, and the like. Descriptions of such techniques can be found in, among other sources, Ausbel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February; 4(1):41-7, U.S. Pat. Nos. 6,027,998; 6,605,451, Barany et al., PCT Publication No. WO 97/31256; Wenz et al., PCT Publication No. WO 01/92579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); Lage et al., Genome Res. 2003 February; 13(2):294-307, and Landegren et al., Science 241:1077-80 (1988), Demidov, V., Expert Rev Mol Diagn. 2002 November; 2(6):542-8., Cook et al., J Microbiol Methods. 2003 May; 53(2):165-74, Schweitzer et al., Curr Opin Biotechnol. 2001 February; 12(1):21-7, U.S. Pat. Nos. 5,830,711, 6,027,889, 5,686,243, PCT Publication No. WO0056927A3, and PCT Publication No. WO9803673A1.

In some embodiments, amplification comprises at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can comprise thermocycling or can be performed isothermally.

Unless otherwise indicated, the term “hybridize” is used herein refer to “specific hybridization” which is the binding, duplexing, or hybridizing of a nucleic acid molecule preferentially to a particular nucleotide sequence, in some embodiments, under stringent conditions. The term “stringent conditions” refers to conditions under which a probe will hybridize preferentially to its target sequence, and to a lesser extent to, or not at all to, other sequences. A “stringent hybridization” and “stringent hybridization wash conditions” in the context of nucleic acid hybridization (e.g., as in array, Southern, or Northern hybridization) are sequence-dependent and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part I, Ch. 2, “Overview of principles of hybridization and the strategy of nucleic acid probe assays,” Elsevier, NY (“Tijssen”). Generally, highly stringent hybridization and wash conditions for filter hybridizations are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe. Dependency of hybridization stringency on buffer composition, temperature, and probe length are well known to those of skill in the art (see, e.g., Sambrook and Russell (2001) Molecular Cloning: A Laboratory Manual (3rd ed.) Vol. 1-3, Cold Spring Harbor Laboratory, Cold Spring Harbor Press, NY).

A “sample,” or “biological sample” as used herein, includes various samples of tissue, cells, or fluid isolated from a subject, including but not limited to, for example, whole blood, buffy coat, plasma, serum, immune cells (e.g., monocytes or macrophages), and sputa. In some embodiments, the sample comprises a buffer, such as an anticoagulant, and/or a preservative. In some embodiments whole blood is mixed with heparin in a lithium heparin blood collection tube. The sample can be from any bodily fluid, tissue or cells that contain the expressed biomarker. A biological sample can be obtained from a subject by conventional techniques. For example, blood can be obtained by venipuncture or a finger-prick capillary, and solid tissue samples can be obtained by surgical techniques according to methods well known in the art. In some aspects, the blood sample is placed into a tube that is specifically designed for the assay.

An “endogenous control,” as used herein refers to a moiety that is naturally present in the sample to be used for detection. In some embodiments, an endogenous control is a “sample adequacy control” (SAC), which may be used to determine whether there was sufficient sample used in the assay, or whether the sample comprised sufficient biological material, such as cells. In some embodiments, an endogenous control is an RNA (such as an mRNA, tRNA, ribosomal RNA, etc.), such as a human RNA. Nonlimiting exemplary endogenous controls include CD3E, TBP, CD4, CD8B, B2M, ABL mRNA, GUSB mRNA, GAPDH mRNA, TUBB mRNA, and UPK1a mRNA. In some embodiments, an endogenous control, such as a SAC, is selected that can be detected in the same manner as the target RNA is detected and, in some embodiments, simultaneously with the target RNA. Controls may be used both for relative quantitation, for example, normalizing gene expression levels of a marker and to establish a Ct cut-off value for specimen stability.

An “exogenous control,” as used herein, refers to a moiety that is added to a sample or to an assay, such as a “sample processing control” (SPC). In some embodiments, an exogenous control is included with the assay reagents. An exogenous control is typically selected that is not expected to be present in the sample to be used for detection, or is present at very low levels in the sample such that the amount of the moiety naturally present in the sample is either undetectable or is detectable at a much lower level than the amount added to the sample as an exogenous control. In some embodiments, an exogenous control comprises a nucleotide sequence that is not expected to be present in the sample type used for detection of the target RNA. In some embodiments, an exogenous control comprises a nucleotide sequence that is not known to be present in the species from whom the sample is taken. In some embodiments, an exogenous control comprises a nucleotide sequence from a different species than the subject from whom the sample was taken. In some embodiments, an exogenous control comprises a nucleotide sequence that is not known to be present in any species. In some embodiments, an exogenous control is selected that can be detected in the same manner as the target RNA is detected and, in some embodiments, simultaneously with the target RNA. In some embodiments, the exogenous control is an RNA. In some such embodiments, the exogenous control is an ARMORED® RNA, which comprises RNA packaged in a bacteriophage protective coat. See, e.g., WalkerPeach et al., Clin. Chem. 45:12: 2079-2085 (1999).

In the present disclosure, the terms “target RNA” and “target gene” are used interchangeably to refer to any of the biomarker genes described herein, as well as to exogenous and/or endogenous controls. Thus, it is to be understood that when a discussion is presented in terms of a target gene, that discussion is specifically intended to encompass the biomarker genes, any endogenous control(s) (e.g., SAC), and any exogenous control(s) (e.g., SPC).

In the sequences herein, “U” and “T” are used interchangeably, such that both letters indicate a uracil or thymine at that position. One skilled in the art will understand from the context and/or intended use whether a uracil or thymine is intended and/or should be used at that position in the sequence. For example, one skilled in the art would understand that native RNA molecules typically include uracil, while native DNA molecules typically include thymine. Thus, where an RNA sequence includes “T”, one skilled in the art would understand that that position in the native RNA is likely a uracil.

In the present disclosure, “a sequence selected from” encompasses both “one sequence selected from” and “one or more sequences selected from.” Thus, when “a sequence selected from” is used, it is to be understood that one, or more than one, of the listed sequences may be chosen.

In the present disclosure, the phrase “level of expression”, “expression level” and “amount” are used interchangeably to refer to the amount of a specific molecule (e.g. a specific RNA transcript) present in a given biological sample or from biological material extracted form a biological sample. The “level of expression”, or “expression level” can refer to expression of either mRNA or protein whose abundance is measured quantitatively.

The phrase “differentially expressed” refers to differences in the quantity and/or the frequency of a biomarker present in a sample taken from patients having, for example, tuberculosis as compared to a control subject or non-infected subject. For example, a biomarker can be a polynucleotide which is present at an elevated level or at a decreased level in samples of patients with MTB, ATB, incipient TB, subclinical TB or LTBI compared to samples of control subjects. Alternatively, a biomarker can be a polynucleotide which is detected at a higher frequency or at a lower frequency in samples of patients with tuberculosis compared to samples of control subjects. A biomarker can be differentially present in terms of quantity, frequency or both.

A polynucleotide is differentially expressed between two samples if the amount of the polynucleotide in one sample is statistically significantly different from the amount of the polynucleotide in the other sample. For example, a polynucleotide is differentially expressed in two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, a polynucleotide is differentially expressed in two sets of samples if the frequency of detecting the polynucleotide in samples of patients infected with MTB, is statistically significantly higher or lower than in the control samples or if patients having ATB is statistically significantly higher or lower than in the samples with incipient TB, subclinical TB or LTBI. For example, a polynucleotide is differentially expressed in two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

A “biomarker” in the context of the present disclosure refers to a biological compound, such as a polynucleotide or polypeptide which is differentially expressed in a sample taken from patients having ATB, ITB or STB as compared to a comparable sample taken from control subjects (e.g., patients with latent tuberculosis or other pulmonary and infectious diseases or non-infected subject). The biomarker can be a nucleic acid, a fragment of a nucleic acid, a polynucleotide, or an oligonucleotide that can be detected and/or quantified. Tuberculosis biomarkers include polynucleotides comprising nucleotide sequences from genes or RNA transcripts of genes, including but not limited to, DUSP3, GBP5, TBP, and their expression products.

The terms “diagnosis” and “diagnostics” also encompass the terms “prognosis” and “prognostics”, respectively, as well as the applications of such procedures over two or more time points to monitor the diagnosis and/or prognosis over time, and statistical modeling based thereupon. Furthermore the term diagnosis includes: a. prediction (determining if a patient will likely develop aggressive disease), b. prognosis (predicting whether a patient will likely have a better or worse outcome at a pre-selected time in the future), c. therapy selection, d. therapeutic drug monitoring, and e. relapse monitoring.

As used herein, the term “treating” or “treat” describes the management and care of a patient for the purpose of combating a disease, condition, or disorder and includes the administration of any of the tuberculosis treatments described herein or any other tuberculosis treatments known in the art, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) preventing the appearance of clinical symptoms of the state or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

As used herein, the term “preventing,” “prevent,” or “protecting against” describes reducing or eliminating the onset of the symptoms or complications of such disease, condition or disorder.

The terms “effective amount” and “therapeutically effective amount” of an agent or compound are used in the broadest sense to refer to a nontoxic but sufficient amount of an active agent or compound to provide the desired effect or benefit.

The term “benefit” is used in the broadest sense and refers to any desirable effect and specifically includes clinical benefit as defined herein. Clinical benefit can be measured by assessing various endpoints, e.g., inhibition, to some extent, of disease progression, including slowing down and complete arrest; reduction in the number of disease episodes and/or symptoms; reduction in lesion size; inhibition (i.e., reduction, slowing down or complete stopping) of disease cell infiltration into adjacent peripheral organs and/or tissues; inhibition (i.e. reduction, slowing down or complete stopping) of disease spread; inhibition (i.e. reduction, slowing down or complete stopping) of the spread of an infection in a subject; decrease of auto-immune response, which may, but does not have to, result in the regression or ablation of the disease lesion; relief, to some extent, of one or more symptoms associated with the disorder; increase in the length of disease-free presentation following treatment, e.g., progression-free survival; increased overall survival; higher response rate; and/or decreased mortality at a given point of time following treatment.

As would be appreciated by the skilled artisan, the term “GBP5” refers to guanylate binding protein 5 and any isoforms thereof. An example of the nucleotide sequence of GBP5 is published in the NCBI database under the accession number CH471097 or AC099063.

As would be appreciated by the skilled artisan, the term “DUSP3” refers to dual specificity phosphatase 3 and any isoforms thereof. An example of the nucleotide sequence of DUSP3 is published in the NCBI database under the accession number CH471178.2 or AC003098.

As would be appreciated by the skilled artisan, the term “TBP” refers to TATA-box binding protein and any isoforms thereof. TBP is a key component of the eukaryotic transcription initiation machinery. It functions in several complexes involved in core promoter recognition and assembly of the pre-initiation complex. Through gene duplication eukaryotes have expanded their repertoire of TATA binding proteins, leading to a variable composition of the transcription machinery. An example of the nucleotide sequence of TBP is published in the NCBI database under the accession number AL031259.1 or AY368204.1.

Identifying Active Tuberculosis Infections

PCR tests have become a widely applied diagnostic technology for detecting active TB infection. But there are numerous critical components in the workflow that need to be accounted for in order to reach biologically meaningful and trustworthy conclusions. Particularly, it is well established that differences in pre-analytical conditions due to inconsistent specimen collection handling, processing, and extraction (including, the type and size of the biological samples, vessel used, duration and temperature of a delay to processing, preservation method, temperature and duration of storage, number of freeze-thaw cycles, and normalization) introduce a pronounced source of bias in any laboratory testing. The problems associated with the collection and handling of biological specimens from which it is desirable to obtain nucleic acids are further exacerbated when the desired nucleic acids for downstream analysis include ribonucleic acid (RNA), which may at times is susceptible to degradation by endogenous or exogenous nuclease activity. Identifying and minimizing effects introduced by preanalytical variability is difficult as such effects are often not global in nature but instead can be specific to the type of biological samples used, the gene, or transcript affected. To avoid pitfalls in data analysis and interpretation of biomarkers in PCR diagnostic tests, the selection of biomarkers and internal controls are crucial.

Herein are disclosed host response type PCR tests that detect a patient's specific transcriptional response for detecting active, subclinical or incipient tuberculosis (ATB, ITB or STB) infection, distinguishing active tuberculosis (ATB) from latent tuberculosis and other pulmonary and infectious diseases, predicting low risk or high risk of developing ATB, monitoring responses to tuberculosis treatment, and predicting progression to ATB from ITB.

The present inventors have developed methods for accurately detecting individuals that are infected with ATB, ITB or STB, and distinguishing active tuberculosis infections from other diseases. The methods disclosed herein utilize a combination of biomarkers having surprisingly similar transcript stability at room temperature, at elevated temperatures, or at lower temperatures over time. The methods comprise measuring expression levels of a set of biomarkers, DUSP3 (dual specificity phosphatase 3), GBP5 (guanylate binding protein 5), and TBP (TATA-box binding protein) and analyzing the set of biomarkers to generate a first signature to diagnose the presence or absence of a tuberculosis infection. The GBP5, DUSP3, and TBP is a self-normalizing host-response signature. Thus, stable expression of the gene signature under investigated conditions is critical in qRT-PCR analysis. For test conditions requiring long stability times, reduction in enzyme activity is tolerated if all targets are equally affected and sample degradation is tolerated if all targets decay with similar kinetics. DUSP3, GBP5, and TBP exhibit low variability in expression stability over time and at different conditions of temperature, which is important in providing accurate and reliable gene expression data. The expression stability for each gene can be determined as delta Ct values from RT-qPCR normalized to a housekeeping gene. The expression stability for the 3 genes (DUSP3, GBP5, and TBP) can be determined as delta TBP score (the terms “delta TBP score” and “TBP score” are used interchangeably herein) according to the equation (GBP5+DUSP3)/2−TBP and as described in the examples. A positive TB-score drift indicates reduced sensitivity (and increased specificity) while a negative TB-score drift indicates reduced specificity (and increased sensitivity). A low variability in expression corresponds to a delta TBP-score that is constant or drifts less than +/−0.5 (less than +/−0.4, less than +/−0.3, or less than +/−0.2) as a function of time and/or temperature.

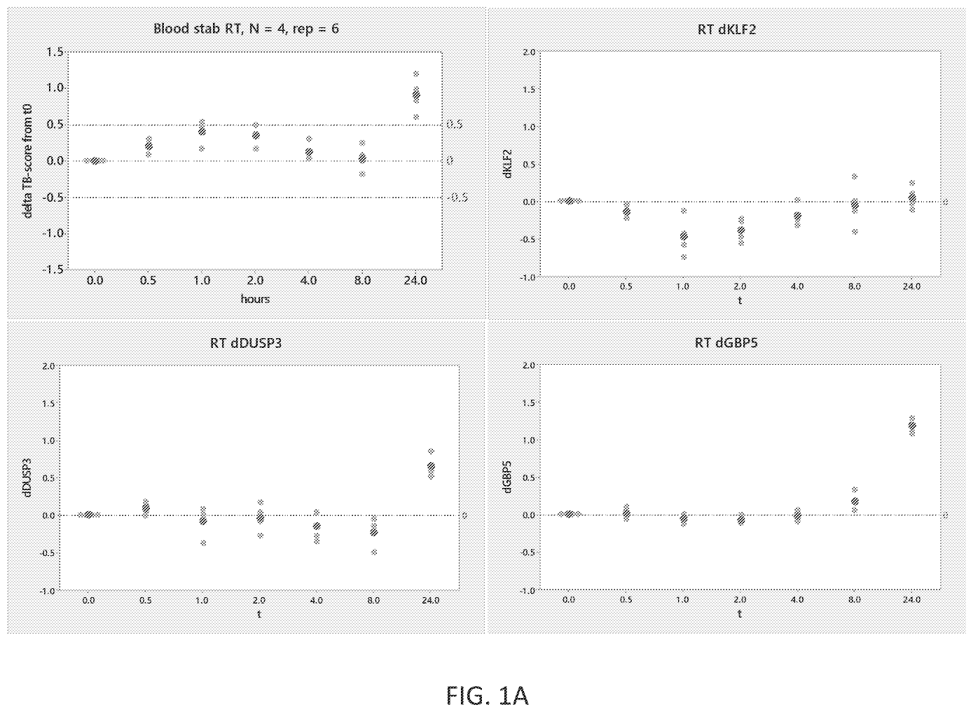

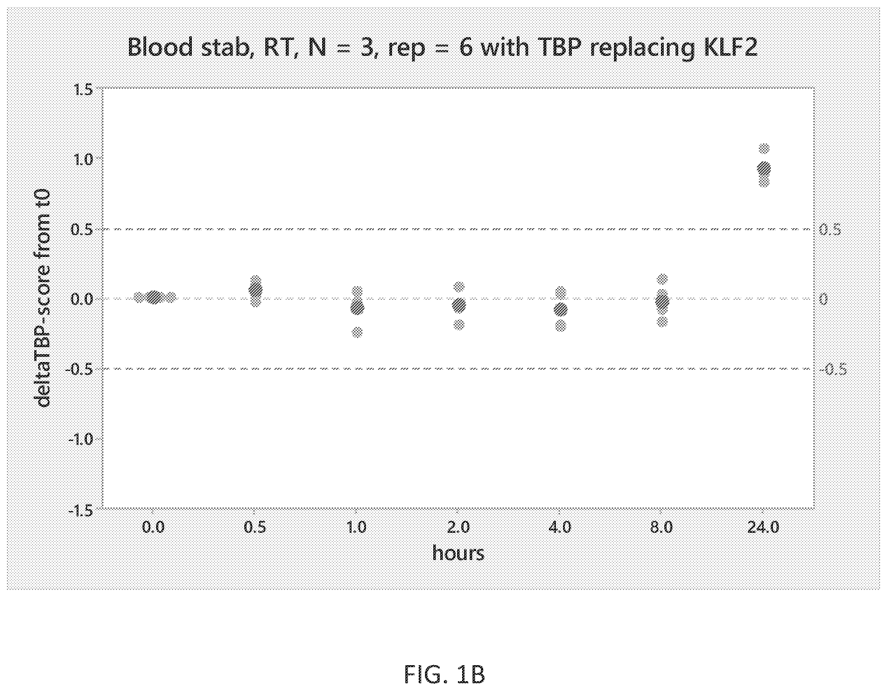

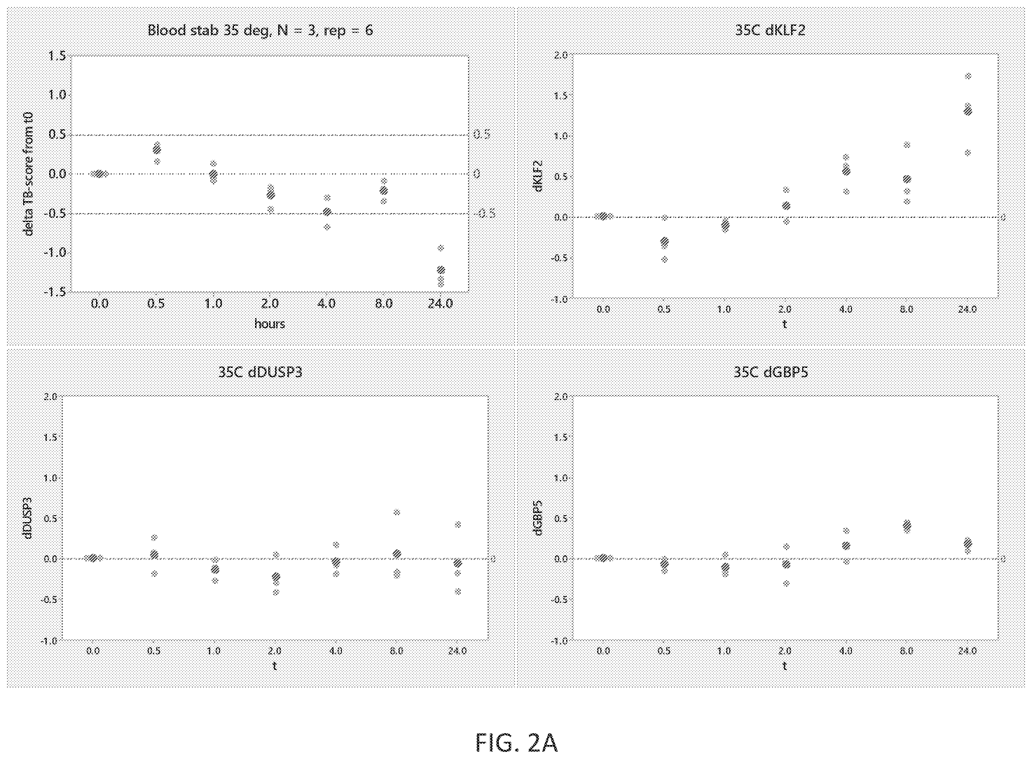

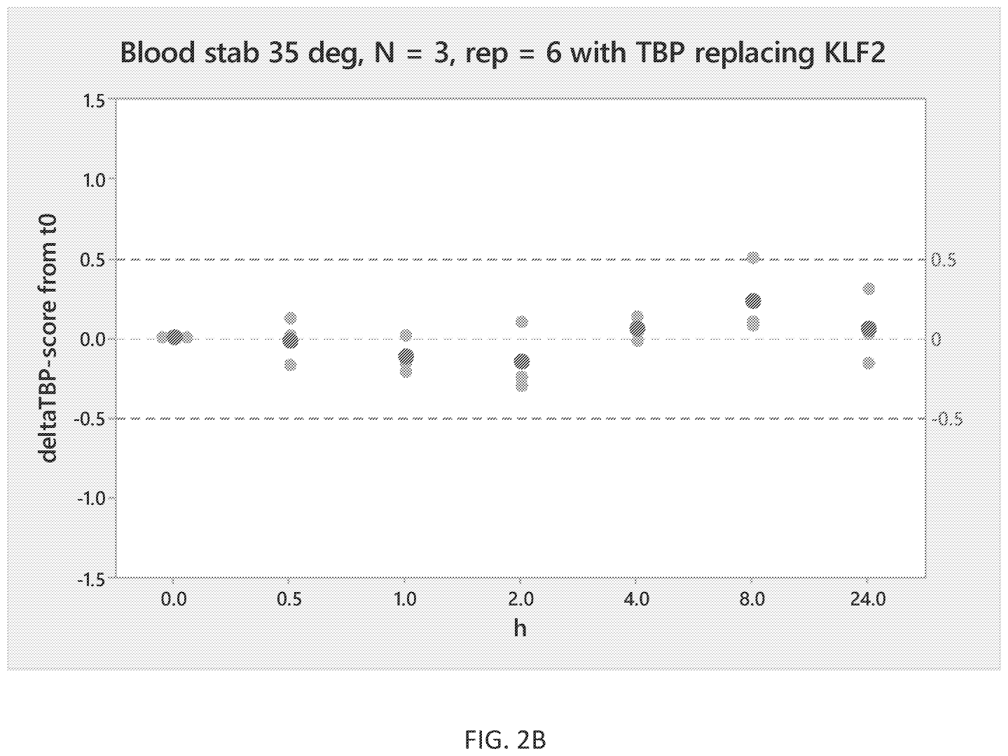

DUSP3, GBP5, and TBP exhibit low variability of expression (a delta TBP-score that is constant or drifts less than +/−0.5) in samples maintained at room temperature, at elevated temperatures (such as 45° C. or greater, 40° C. or greater, 35° C. or greater, 30° C. or greater, or 27° C. or greater), or lower temperatures (such as 23° C. or less, 20° C. or less, 15° C. or less, 10° C. or less, or 5° C. or less). DUSP3, GBP5, and TBP also exhibit low variability of expression in samples maintained at said temperatures for a period of 1 hr or greater, 2 hrs or greater, 5 hrs or greater, 8 hrs or greater, 12 hrs or greater, 18 hrs or greater, or 24 hrs or greater. The changes in expression levels of each biomarker described herein relative to reference value ranges of the biomarker for a control subject are indicative of tuberculosis. For example, an increased level of expression of GBP5 or DUSP3 compared to the reference value ranges for the biomarkers for a control subject indicates that the patient has active tuberculosis. The expression level of TBP relative to reference value ranges of the biomarker for a control subject can be constant or decreased in patients that has tuberculosis. TBP has surprisingly shown a similar variability of expression (or similar degradation rates) as GBP5 and DUSP3 over time and temperature compared to other biomarkers. The combination of GBP5, DUSP3, and KLF2 is a self-normalizing signature, since KLF2 can act as a housekeeping gene. Thus, stable expression of the housekeeping gene under investigated conditions is critical in qRT-PCR analysis. For test conditions requiring long stability times, reduction in enzyme activity is tolerated if all targets are equally affected and sample degradation is tolerated if all targets decay with similar kinetics. FIGS. 1 and 2, for example, show delta TB-score ((GBP5+DUSP3)/2−KLF2) varied significantly with time at RT and at 35° C. while delta TBP-score ((GBP5+DUSP3)/2−TBP) was more stable with time at the same temperatures. All targets in the GBP5, DUSP3, and TBP gene-signature decay with the same kinetics. In contrast, the targets in the GBP5, DUSP3, and KLF2 gene-signature do not decay with same kinetics. KLF2 and other biomarkers (data not shown for other markers) relative to GBP5 and DUSP3 exhibit variability in expression likely due to differences in degradation rates when a sample is held at room or elevated temperature over time. Overall, KLF2 mRNA decay with a different kinetics relative to GBP5 and DUSP3 compared to TBP mRNA which decay with a similar kinetics relative to GBP5 and DUSP3, with time or temperature. The variability in degradation significantly impact the sensitivity and/or specificity of the resulting analysis.

As described herein, the methods comprise measuring levels of mRNA expression of DUSP3, GBP5, and TBP biomarkers and analyzing the expression levels to generate a first signature to diagnose the presence or absence of a tuberculosis infection. The biomarkers can be a nucleic acid, a fragment of a nucleic acid, a polynucleotide, or an oligonucleotide that can be detected and/or quantified. Biomarkers that can be used in the practice of the disclosure include polynucleotides comprising nucleotide sequences from genes or RNA transcripts of genes, including but not limited to, DUSP3, GBP5, and TBP, and their expression products. Differential expression of these biomarkers is associated with tuberculosis and therefore expression profiles of these biomarkers are useful for diagnosing tuberculosis infection and determining the disease stage of those individuals that are infected with tuberculosis.

Differential expression may be measured by comparing the Ct of the biomarker to the Ct of a control or reference marker to obtain a ΔCt value. When analyzing the levels of biomarkers in a biological sample, the reference value ranges used for comparison can represent the levels of one or more biomarkers found in one or more samples of one or more subjects without active tuberculosis (e.g., healthy subject, non-infected subject, or subject with latent tuberculosis). Alternatively, the reference value ranges can represent the levels of one or more biomarkers found in one or more samples of one or more subjects with active tuberculosis. In certain embodiments, the levels of the biomarkers in a biological sample from a subject are compared to reference values for subjects with active tuberculosis, incipient tuberculosis, or subclinical tuberculosis.

In addition to the biomarkers GBB5, DUSP3, and TBP, diagnosis of tuberculosis can further comprise measuring and analyzing expression of at least 1 additional biomarker and up to 30 total biomarkers, including any number of biomarkers in between, such as 2, 3, 4, 5, 6, 7, 8, 9, 10, 11, 12, 13, 14, 15, 16, 17, 18, 19, 20, 21, 22, 23, 24, 25, 26, 27, 28, 29, or 30 biomarkers. In certain embodiments, the disclosure includes a biomarker panel comprising at least 2, at least 3, or at least 4, or at least 5, or at least 6, or at least 7, or at least 8, or at least 9, or at least 10, or at least 11 or more biomarkers. Although smaller biomarker panels are usually more economical, larger biomarker panels (i.e., greater than 30 biomarkers) have the advantage of providing more detailed information and can also be used in the practice of the disclosure.

In some embodiments, the expression levels of each of a first panel of biomarkers is analyzed to diagnose the patient as having active tuberculosis or not infected and optionally, the expression levels of each of a second panel of biomarkers can be analyzed to diagnose the patient as having active tuberculosis, incipient tuberculosis, or subclinical tuberculosis. The first and second panel may share 1 or more biomarkers in common or there may be not overlap between the panels. For example, GBP5, DUSP3, and/or TBP may be included in both panels. For biomarkers that are common to both signatures they may be weighted differently in the first and second signatures. To provide the signature, the methods can include comparing the expression level of the biomarkers to a reference value for that biomarker or to a control.

In certain embodiments, the combined set of biomarkers that are measured and analyzed for expression level include DUSP3, GBP5, and TBP. The additional biomarkers that can be analyzed (in the first signature or the second signature) can be selected from ACTB, ANKRD22, B2M, CDC37, CISH, CCL7, DECR1, DUSP3, EEF1A1, FAM48A, FLT1, FoxP3, GAPDH, GBP1P1, GBP5, HPRT1, IFNg, IP10, IL2, IL10, IL2RA, IL8, IL12B, KLF2, LINC01093, MIG, PLAU, PRDX1, PTGS2, RAB8B, RPLP0, SERPING1, SIRT5, SLPI, TBP, TNFA, TGFA, TRAP1, UBC, UBE2D2, VEGFA, or YWHAZ. In some embodiments, only a first panel of biomarkers is measured and analyzed to diagnose the patient as having active tuberculosis, incipient tuberculosis, subclinical tuberculosis, distinguishing among active tuberculosis infections from other diseases, monitor progression of tuberculosis, or monitor tuberculosis treatment and the biomarkers include DUSP3, GBP5, and TBP. Optionally, a second panel of biomarkers can be measured and analyzed to provide a second signature, wherein the biomarkers can be selected from one or more of DUSP3, GBP5, TBP, IFNg, MIG, IP10, IL2, FoxP3, PLAU, SLPI, VEGFA, GBP1P1, ANKRD22, SERPING1, PTGS2, IL10, TNFA, TGFA, IL2RA, IL8, IL12B, CISH, FLT1, LINC01093, KLF2, PRDX1, or CCL7.

In some embodiments, a single assay is performed to measure the expression levels of each biomarker and the data is used to generate a value for a gene signature. A cut off value or “score” from the signature may be used to diagnose the patient as having ATB or not infected, a second cutoff value may be used to diagnose the patient as having ITB or not, and/or a third cutoff value may be used to diagnose the patient as having STB or not. Markers within the signature may be given different weights to calculate the “score”. Additional clinical data such as risk assessment, radiography and other clinical and laboratory findings may also be incorporated into the determination of the score. In some aspects, the scores may be reported in different ways depending on the clinical setting. In some aspects the score may be differentially weighted depending on the clinical setting from which the sample was collected. For example, the analysis may vary depending on whether a clinician or a self-collected sample is used, and based on the availability of treatment and follow-up provided by the clinic.

The cut-off values may be modified depending on the need. For example, if the intent is to rule out ATB for TB preventive treatment or to rule in ATB for full-course TB treatment different cut-off values may be selected. In some embodiments, the methods are used to monitor treatment response. A shift in the results of the signature analysis from the ATB group toward the not ATB group could be used as an indication of patient improvement.

In some aspects, the methods described herein may be used to determine if a patient should receive a full-course of treatment for ATB or another treatment appropriate for the status of the patient's infection. For example, a patient is selected for treatment for tuberculosis if the patient has a diagnosis of ATB based on a biomarker expression profile as described herein. The methods described herein may be used to monitor progression and predict likelihood of progression to ATB for patients identified as having ITB or STB or to predict/monitor treatment response to determine when infection has been eliminated/quiescent or when ATB has reverted to stable LTBI or has been eliminated/quiescent. Patients with ITB can progress to STB and then to ATB through increased disease burden.

Accordingly, the disclosure includes a method of treating a subject having TB (ATB, STB or ITB), the method comprising: diagnosing the subject with TB according to a method described herein; and administering a therapeutically effective amount of at least one tuberculosis treatment to the subject if the subject has a positive tuberculosis diagnosis. In another embodiment, the disclosure includes a method of treating a subject suspected of having an ATB infection, the method comprising: receiving information regarding the diagnosis of the subject according to a method described herein; and administering a therapeutically effective amount of at least one tuberculosis treatment to the subject if the patient has a positive ATB infection.

In some aspects, the at least one tuberculosis treatment comprises at least one antibiotic, at least one corticosteroid, or any combination thereof.

Accordingly, the disclosure includes a method of treating a subject having TB (ATB, STB or ITB), the method comprising: diagnosing the subject with TB according to a method described herein; and administering a therapeutically effective amount of at least one antibiotic to the subject if the subject has a positive tuberculosis diagnosis. In another embodiment, the disclosure includes a method of treating a subject suspected of having an ATB infection, the method comprising: receiving information regarding the diagnosis of the subject according to a method described herein; and administering a therapeutically effective amount of at least one antibiotic to the subject if the patient has a positive ATB infection.

Antibiotics that may be used in treating tuberculosis include, but are not limited to, ethambutol, isoniazid, pyrazinamide, rifabutin, rifampicin, rifapentine, amikacin, capreomycin, cycloserine, ethionamide, levofloxacin, moxifloxacin, para-aminosalicylic acid, and streptomycin. Typically, several antibiotics are administered simultaneously to treat active tuberculosis, whereas typically a single antibiotic is administered to treat latent tuberculosis. Treatment may continue for at least a month or several months, up to one or two years, or longer, depending on whether the tuberculosis infection is active, subclinical, incipient or latent. Longer treatment is generally required for severe tuberculosis infection, particularly if the infection becomes antibiotic resistant. Subjects, whose infection is antibiotic resistant, may be screened to determine antibiotic sensitivity in order to identify antibiotics that will eradicate the tuberculosis infection. In addition, corticosteroid medicines also may be administered to reduce inflammation caused by active tuberculosis.

A recommended method to treat ITB can include the conventional regimens for LTBI, which include 6 to 12 months of isoniazid, 3 months of a combination of rifamycin and isoniazid, or 3 to 4 months of a rifamycin alone, may be effective for ITB, assuming a relatively low burden of disease. Other methods to treat ITB regardless of HIV status provides 6 to 9 months of daily isoniazid, or a 3 month regimen of weekly rifapentine plus isoniazid, or a 3 month regiment of daily isoniazid plus rifampicin. A 1 month regimen of daily rifapentine plus isoniazid or 4 months of daily rifampicin alone may also be provided as an alternative. In settings with high TB transmission, for example in adults and adolescents living with HIV who have an unknown or a positive ITB test but not diagnosed with ATB disease, the patient may receive at least 36 months of daily isoniazid therapy. Newer, existing, and repurposed drugs (bedaquiline, delamanid, linezolid, likely sutezolid in the future, and fluoroquinolones) are now being used in clinical practice to treat drug-resistant TB. Another treatment option is providing a high dose of rifamycins, which have the potential to shorten the duration of conventional treatment for active TB and are well tolerated. Clinical trials are in progress to evaluate the utility of different drugs, including delamanid and fluoroquinolones, for the treatment of multidrug-resistant LTBI. If successful, then they may also be appropriate for treating ITB. Other alternative approaches for the treatment of ITB might include host-directed therapies or combining immunosuppressive agents and anti-TB drugs.

Treatment of STB can include concurrent HIV testing and, where possible, obtaining a biological sample for drug susceptibility testing. The presence of HIV coinfection will impact the choice of antiretroviral therapy and raise considerations related to immune reconstitution inflammatory syndrome, while regimens for the treatment of drug-resistant TB will depend on the susceptibility profile of the M. tuberculosis isolate. However, in general, treatment for STB can be identical to that of conventional active TB disease while taking into account comorbidities, potential drug-drug interactions, and other considerations described above. Persons with ITB or STB may be started on isoniazid preventive therapy (IPT).

The methods of the disclosure, as described herein, can also be used for determining the prognosis of a subject and for monitoring treatment of a subject who has tuberculosis. A medical practitioner can monitor the progress of disease by measuring the levels of the biomarkers in biological samples from the patient.

The methods described herein may be used as part of a tuberculosis triage test. A TB triage test is designed to be used in adults and children identified as having symptoms compatible with TB or having risk factors for any form of active TB (or at a minimum for pulmonary TB). Triage testing should stratify individuals for either confirmatory TB diagnostic testing (for triage test-positive patients) or further investigation of likely non-TB aetiologias (for triage test-negative patients). For example, confirmatory TB diagnostic testing for triage test-positive patients can be performed and include the GENEXPERT® system or other WHO endorsed confirmatory tests such as mycobacterial culture. Treatment can be initiated while awaiting results from the confirmatory TB test. For triage test-negative patients, further tests for other respiratory diseases can be performed. The key characteristics defined for a TB triage test at a WHO consensus meeting to develop target product profiles (TPPs) for new TB diagnostic tests in 2014 were that it should be: non-sputum based; easy to use; rapid; accurate (optimally 95% sensitive and 80% specific for any form of active TB when compared with the confirmatory test, or minimally 90% sensitive and 70% specific for pulmonary TB when compared with the confirmatory test); affordable; and usable with only minimal infrastructure and training needs. The compositions, kits, apparatus, and methods disclosed herein provide an optimized triage test for TB.

The methods described herein may be used for diagnosing extrapulmonary tuberculosis. An Mtb infection is often a pulmonary infection. However, dissemination of tuberculosis outside of lungs can lead to the appearance of a number of uncommon findings with characteristic patterns that include skeletal tuberculosis, genital tract tuberculosis, urinary tract tuberculosis, central nervous system (CNS) tuberculosis, gastrointestinal tuberculosis, adrenal tuberculosis, scrofula, and cardiac tuberculosis. Thus, an MtB infection can also be extrapulmonary. Extrapulmonary sites of infection commonly include lymph nodes, pleura, and osteoarticular areas, although any organ can be involved. The diagnosis of extrapulmonary tuberculosis often is elusive. Generally, children and subject who are immunosuppressed are susceptible to extra-pulmonary Mtb infections.

Lymphadenitis is the most commonly occurring form of extrapulmonary tuberculosis. Cervical adenopathy is most common, but inguinal, axillary, mesenteric, mediastinal, and intramammary involvement all have been described. Pleural tuberculosis often is an acute illness with cough, pleuritic chest pain, fever, or dyspnea. Bone and joint tuberculosis may account for up to 35 percent of cases of extrapulmonary tuberculosis. Skeletal tuberculosis most often involves the spine, followed by tuberculous arthritis in weight-bearing joints and extraspinal tuberculous osteomyelitis. Central nervous system tuberculosis includes tuberculous meningitis (the most common presentation), intracranial tuberculomas, and spinal tuberculous arachnoiditis. Abdominal tuberculosis may involve the gastrointestinal tract, peritoneum, mesenteric lymph nodes, or genitourinary tract. Miliary tuberculosis, tuberculous pericarditis, and tuberculosis associated with tumor necrosis factor-alpha (TNF-alpha) inhibitors are additional forms of extra-pulmonary tuberculosis.

A six- to nine-month regimen (two months of isoniazid, rifampin, pyrazinamide, and ethambutol, followed by four to seven months of isoniazid and rifampin) is recommended as initial therapy for all forms of extrapulmonary tuberculosis unless the organisms are known or strongly suspected to be resistant to the first-line drugs.

The methods described herein for prognosis or diagnosis of subjects who have tuberculosis may be used in individuals who have not yet been diagnosed (for example, preventative screening), or who have been diagnosed, or who are suspected of having tuberculosis (e.g., display one or more characteristic symptoms), or who are at risk of developing tuberculosis (e.g., have a genetic predisposition or presence of one or more developmental, environmental, or behavioral risk factors). For example, patients having one or more risk factors including, but not limited to, patients who are immunosuppressed, immunodeficient, elderly, suspected of having had exposure to a subject infected with tuberculosis, or having symptoms of lung disease may be screened by the methods described herein. The methods may also be used to evaluate severity of disease. The methods may also be used to detect the response of tuberculosis to therapeutic treatments or other interventions (e.g., worsening, status-quo, partial recovery, or complete recovery) of the patient, and the appropriate course of action, resulting in either further treatment or observation, or in discharge of the patient from the medical care center.

In one embodiment, the disclosure includes methods for diagnosing and treating a patient suspected of being infected with TB. The methods can comprise obtaining a biological sample from the patient and measuring levels of expression of the biomarkers DUSP3, GBP5, and TBP in the biological sample. The levels of expression of each biomarker can be analyzed in conjunction with respective reference value ranges for each biomarker. Similarity of the levels of expression of the biomarkers to reference value ranges for a subject with active tuberculosis indicates that the patient has active tuberculosis, similarity of the levels of expression of the biomarkers to reference value ranges for a subject with subclinical tuberculosis indicates that the patient has subclinical tuberculosis, and similarity of the levels of expression of the biomarkers to reference value ranges for a subject with incipient tuberculosis indicates that the patient has incipient tuberculosis.

The methods can include measuring levels of expression of additional biomarkers selected from ACTB, ANKRD22, B2M, CDC37, CISH, CCL7, DECR1, DUSP3, EEF1A1, FAM48A, FLT1, FoxP3, GAPDH, GBP1P1, GBP5, HPRT1, IFNg, IP10, IL2, IL10, IL2RA, IL8, IL12B, KLF2, LINC01093, MIG, PLAU, PRDX1, PTGS2, RAB8B, RPLP0, SERPING1, SIRT5, SLPI, TBP, TNFA, TGFA, TRAP1, UBC, UBE2D2, VEGFA, or YWHAZ biomarkers in the biological sample. Different combinations of biomarkers may be analyzed depending on determinations desired.

In one embodiment, DUSP3, GBP5, and TBP are used in a signature to distinguish ATB, ITB, and STB from other diseases. In one embodiment, DUSP3 and GBP5 are used in a signature to distinguish among ATB, ITB and STB from other diseases.

In one embodiment, DUSP3, GBP5, and TBP are used in a signature to monitor treatment response of ATB patients. In one embodiment, DUSP3 and GBP5 are used in a signature to monitor treatment response of ATB patients.

The present disclosure provides methods for diagnosing tuberculosis in a patient, the methods comprising: a) measuring levels of expression of DUSP3, GBP5, and TBP biomarkers in a biological sample from the patient; b) determining a score based on the levels of expression of the DUSP3, GBP5 and TBP biomarkers, wherein the score is calculated using the formula:

$$Score = \frac{( GBP 5 + DUSP 3 )}{2} - TBP$$

wherein GBP5 is the level of expression of the GBP5 biomarker measured in step (a), DUSP3 is the level of expression of the DUSP3 biomarker measured in step (a) and TBP is the level of expression of the TBP biomarker measured in step (a); and c) identifying that the patient has tuberculosis or does not have tuberculosis based on the score. In some aspects, the methods can further comprise administering to a patient identified as having tuberculosis an effective amount of at least one tuberculosis treatment, wherein the at least one tuberculosis treatment comprises at least one antibiotic, at least one corticosteroid or any combination thereof.

The present disclosure provides methods for treating tuberculosis in a patient, the methods comprising: a) measuring levels of expression of DUSP3, GBP5, and TBP biomarkers in a biological sample from the patient; b) determining a score based on the levels of expression of the DUSP3, GBP5 and TBP biomarkers, wherein the score is calculated using the formula:

$$Score = \frac{( GBP 5 + DUSP 3 )}{2} - TBP$$

wherein GBP5 is the level of expression of the GBP5 biomarker measured in step (a), DUSP3 is the level of expression of the DUSP3 biomarker measured in step (a) and TBP is the level of expression of the TBP biomarker measured in step (a); and c) identifying that the patient has tuberculosis or does not have tuberculosis based on the score; and d) administering to a patient identified as having tuberculosis an effective amount of at least one tuberculosis treatment, wherein the at least one tuberculosis treatment comprises at least one antibiotic, at least one corticosteroid or any combination thereof.

In some aspects of the preceding methods, the at least one antibiotic is selected from the group consisting of rifampicin, isoniazid, pyrazinamide, ethambutol, rifapentine, ethionamide, moxifloxacin, and streptomycin.

In some aspects of the preceding methods, step (c) can comprise comparing the score to a predetermined cutoff value. In some aspects, the patient is identified as having tuberculosis when the score is greater than or equal to the predetermined cutoff value and the patient is identified as not having tuberculosis when the score is less than the predetermined cutoff value. In some aspects, the patient is identified as having tuberculosis when the score is less than or equal to the predetermined cutoff value and the patient is identified as not having tuberculosis when the score is greater than the predetermined cutoff value.

In some aspects, a predetermined cutoff value can distinguish between active tuberculosis, incipient tuberculosis, and subclinical tuberculosis infected patients, high risk for tuberculosis, low risk for tuberculosis and TB negative.

In some aspects, a predetermined cutoff value can have a specificity of at least 85%, or at least 90%, or at least 95%, or at least 99.5%.

In some aspects, a predetermined cutoff value can have a sensitivity of at least 85%, or at least 90%, or at least 95%, or at least 99.5%.

In some aspects, a predetermined cutoff value can have a positive predictive value of at least 85%, or at least 90%, or at least 95%, or at least 99.5%.

In some aspects, a predetermined cutoff value can have a negative predictive value of at least 85%, or at least 90%, or at least 95%, or at least 99.5%.

Exemplary predetermined cutoff values can range from about −6 to about 2, including, but not limited to, about −6, about −5.5, about −5, about −4.5, about −4, about −3.5, about −3, about −2.5, about −2, about −1.5, about −1, about −0.5, about 0, about 0.5, about 1, about 1.5, or about 2.

Biomarker data may be analyzed by a variety of methods to identify biomarkers and determine the statistical significance of differences in observed levels of expression of the biomarkers between test and reference expression profiles in order to distinguish a patient with ATB, ITB or STB from having other diseases.

In certain embodiments, patient data is analyzed by one or more methods including, but not limited to, multivariate linear discriminant analysis (LDA), receiver operating characteristic (ROC) analysis, principal component analysis (PCA), random forest, support vector machines, elastic net methods, ensemble data mining methods, significance analysis of microarrays (SAM), cell specific significance analysis of microarrays (csSAM), spanning-tree progression analysis of density-normalized events (SPADE), and multi-dimensional protein identification technology (MUDPIT) analysis. (See, e.g., Hilbe (2009) Logistic Regression Models, Chapman & Hall/CRC Press; McLachlan (2004) Discriminant Analysis and Statistical Pattern Recognition. Wiley Interscience; Zweig et al. (1993) Clin. Chem. 39:561-577; Breiman (2001) Random forests, Machine Learning 45:5032; Pepe (2003) The statistical evaluation of medical tests for classification and prediction, New York, N.Y.: Oxford; Sing et al. (2005) Bioinformatics 21:3940-3941; Tusher et al. (2001) Proc. Natl. Acad. Sci. U.S.A. 98:5116-5121; Oza (2006) Ensemble data mining, NASA Ames Research Center, Moffett Field, Calif., USA; English et al. (2009) J. Biomed. Inform. 42(2):287-295; Zhang (2007) Bioinformatics 8: 230; Shen-Orr et al. (2010) Journal of Immunology 184:144-130; Qiu et al. (2011) Nat. Biotechnol. 29(10):886-891; Ru et al. (2006) J. Chromatogr. A. 1111(2): 166-174, Jolliffe Principal Component Analysis (Springer Series in Statistics, 2.sup.nd edition, Springer, N Y, 2002), Koren et al. (2004) IEEE Trans Vis Comput Graph 10:459-470; herein incorporated by reference in their entireties.)

The present assay relies on the polymerase chain reaction (PCR) and can be carried out in a substantially automated manner using a commercially available nucleic acid amplification system. Exemplary nonlimiting nucleic acid amplification systems that can be used to carry out the methods of the disclosure include the GENEXPERT® system, GENEXPERT® Infinity system, and GENEXPERT® Xpress System (Cepheid, Sunnyvale, Calif.). The amplification system may be available at the same location as the individual to be tested, such as a health care provider's office, a clinic or a community hospital, so processing is not delayed by a requirement to transport the sample to another facility. The present assay can be completed in under 3 hours, in some embodiments, under 2 hours, in some embodiments, under 1 hour, in some embodiments, under 45 minutes, in some embodiments, under 35 minutes, and in some embodiments, under 30 minutes, using an automated system, for example, the GENEXPERT® system.

General Methods

In some aspects of the methods of the present disclosure, determining the expression level of a biomarker (e.g. DUSP3, GBP5, and TBP) can comprise performing quantitative PCR (qPCR) on nucleic acids extracted from a biological sample from a subject. As would be appreciated by the skilled artisan, in aspects wherein quantitative PCR is used to quantify the expression level of a biomarker, the expression level of a biomarker can be expressed as a cycle threshold (Ct) value.

As would be appreciated by the skilled artisan, a non-limiting example of a quantitative PCR method includes reverse transcriptase quantitative PCR, RT-qPCR.

As would be appreciated by the skilled artisan, the methods described herein can be used in combination with the GENEXPERT® system (Cepheid, Sunnyvale). The GENEXPERT® system can be used to determine the expression levels of the biomarkers recited herein. As would be appreciated by the skilled artisan, the GENEXPERT® system utilizes a self-contained, single use cartridge. Sample extraction, amplification, and detection may all be carried out within this self-contained “laboratory in a cartridge.” (See e.g., U.S. Pat. Nos. 5,958,349; 6,403,037; 6,440,725; 6,783,736; 6,818,185; 9,873,909; and 10,562,030; each of which is herein incorporated by reference in its entirety.)

Components of the cartridge include, but are not limited to, processing chambers containing reagents, filters, and capture technologies useful to extract, purify, and amplify target nucleic acids. A valve enables fluid transfer from chamber to chamber and contain nucleic acids lysis and filtration components. An optical window enables real-time optical detection. A reaction tube enables very rapid thermal cycling.

In certain aspects, the cartridge can include one or more cartridge bodies comprising a plurality of chambers in fluidic communication, and a nucleic acid-binding substrate for binding nucleic acid in fluidic communication with the processing chambers, wherein the processing chambers comprise reagents for lysing cells from a sample, amplification and detection of nucleic acid from the sample, and a composition comprising sets of primers for detecting GBP5, DUSP3, and TBP. In specific aspects, the plurality of processing chambers comprises a lysis chamber in fluidic communication with the nucleic acid-binding substrate, wherein the lysis chamber comprises one or more reagents for lysing cells, and one or more reaction vessels in fluidic communication with the lysis chamber and configured for amplification of nucleic acid and detection of amplification products. The sets of primers for detecting GBP5, DUSP3, and TBP can be disposed in the one or more reaction vessels.

The plurality of processing chambers may further include a sample chamber for receiving a sample and having at least a fluid outlet in fluid communication with another chamber of the plurality of processing chambers. Optionally, wherein the sample chamber and lysis chamber are the same.

The reaction vessel can be configured to detect a single amplification product or a plurality of amplification products.

In some aspects, after a sample is added to the cartridge, the sample can be optionally contacted with lysis buffer and released nucleic acid, which may be RNA, DNA and/or cDNA can be bound to a nucleic acid-binding substrate such as a silica or glass substrate. The lysis buffer generally comprises a chaotropic agent, a chelating agent, a buffer, a detergent, or a combination thereof. The chaotropic agent can be selected from guanidinium thiocyanate, guanidinium hydrochloride, alkali perchlorate, alkali iodide, urea, formamide, or a combination thereof. The concentration of the chaotropic agent can range from about 1 M to about 10 M, such as from about 2.5 M to about 7.5 M, less than 4.5 M, less than 2 M, or less than 1 M. The chelating agent can be selected from N-acetyl-L-cysteine, ethylenediaminetetraacetic acid (EDTA), diethylene triamine pentaacetic acid (DTPA), ethylenediamine-N,N′-disuccinic acid (EDDS), 1,2-bis(o-aminophenoxy)ethane-N,N,N′,N′-tetraacetic acid (BAPTA), and a phosphonate chelating agent. The concentration of the chelating agent can range from about 10 mM to about 100 mM and/or comprises about 0.5% to about 5% of the lysis reagent. The buffer can be selected from the group consisting of Tris, phosphate buffer, PBS, citrate buffer, TAPS, Bicine, Tricine, TAPSO, HEPES, TES, MOPS, PIPES, Cacodylate, SSC, and MES. The concentration of the buffer can range from about 5 mM to about 100 mM, such as from about 5 mM to about 50 mM. The detergent can be selected from an ionic detergent or a non-ionic detergent. In some examples, the detergent comprises a detergent selected from the group consisting of N-lauroylsarcosine, sodium dodecyl sulfate (SDS), cetyl methyl ammonium bromide (CTAB), TRITON®-X-100, n-octyl-β-D-glucopyranoside, CHAPS, n-octanoylsucrose, n-octyl-β-D-maltopyranoside, n-octyl-β-D-thioglucopyranoside, PLURONIC® F-127, TWEEN® 20, and n-heptyl-β-D-glucopyranoside. The detergent can comprise about 0.1% to about 2% of the lysis reagent, and/or ranges from about 10 mM up to about 100 mM. The lysis reagent can have a pH ranging from about pH 3.0 to about pH 5.5. In some examples, the lysis buffer comprises a guanidinium compound, optionally EDTA, a buffer, and a detergent.

As described herein, the nucleic acid can be bound to a nucleic acid-binding substrate, also referred to herein as a filter. In some examples, the filter comprises glass fibers and optionally a polymeric binder. The glass fibers may be modified with a nucleic acid binding ligand such as an alkylamine, a cycloalkylamine, an alkyloxy amine, a polyamine moiety, an arylamine, an intercalating agent, a DNA groove binder, a peptide, an amino acid, a protein, or a combination thereof. In some examples, the filter comprises a 500 micron to 2000 microns thick glass fiber disk having a pore size of 0.2 microns to 1 micron. In some aspects, the sample can be contacted with a binding reagent, wash reagent, or a combination during or after lysis. The binding reagent can promote binding of nucleic acids to the filter, facilitating the removal of non-target material. In some embodiments, the binding reagent can include a binding polymer such as polyacrylic acid (PAA), polyacrylamide (PAM), polyethylene glycol (PEG), poly(sulfobetaine), or a salt, or combinations thereof. In some embodiments, the filtering reagent and/or the washing reagent can include the binding reagent. For example, the binding reagent, the filtering reagent, and/or the washing reagent can include a binding polymer (e.g., PEG 200), buffer, inorganic salt(s), antioxidant and/or chelating agent, antifoam SE15, sodium azide, disaccharide or disaccharide derivative, carrier protein, a chaotropic agent (such as guanidium hydrochloride) detergent, DMSO, or a combination thereof. The binding polymer can be present in an amount of at least 10% v/v, at least 20% v/v, at least 30% v/v, and/or less than 60% v/v, less than 40% v/v, less than 30% v/v, less than 20% v/v, or less than 10% v/v or can fall within any range bounded by any of these values, e.g., from 10% to 60% v/v, of the binding reagent, filtering reagent, and/or the washing reagent. The buffer can be selected from the group consisting of Tris, 2-amino-2-hydroxymethyl-1,3-propanediol, HEPES, phosphate buffer, PBS, citrate buffer, TAPS, Bicine, Tricine, TAPSO, HEPES, TES, MOPS, PIPES, Cacodylate, SSC, and MES. The concentration of the buffer can range from about 5 mM to about 100 mM, such as from about 5 mM to about 50 mM. The salt, such as NaCl, KCl, or MgCl₂, can be present at a concentration from about 0.05 M to about 1 M, such as from about 0.1 M to about 0.5 M. The antioxidant and/or chelating agent comprises an agent selected from the group consisting of N-acetyl-L-cysteine, ethylenediaminetetraacetic acid (EDTA), diethylene triamine pentaacetic acid (DTPA), ethylenediamine-N,N′-disuccinic acid (EDDS), 1,2-bis(o-aminophenoxy)ethane-N,N,N′,N′-tetraacetic acid (BAPTA), and a phosphonate chelating agent. In some embodiments the antioxidant and/or chelating agent comprises EDTA. In certain embodiments the antioxidant and/or chelating agent comprise 0.2% to about 5%, about 0.2% to about 3%, or about 0.5% to about 2%, or about 0.5% of the binding reagent, filtering reagent, and/or the washing reagent. In some embodiments the concentration of the antioxidant and/or chelating agent in the binding reagent, filtering reagent, or the washing reagent ranges from about 2 mM to about 50 mM or about 5 mM to about 20 mM. In some embodiments, the detergent is an ionic detergent or a non-ionic detergent. The detergent can be selected from an ionic detergent or a non-ionic detergent. In some examples, the detergent comprises a detergent selected from the group consisting of N-lauroylsarcosine, sodium dodecyl sulfate (SDS), cetyl methyl ammonium bromide (CTAB), TRITON®-X-100, n-octyl-β-D-glucopyranoside, CHAPS, n-octanoylsucrose, n-octyl-β-D-maltopyranoside, n-octyl-β-D-thioglucopyranoside, PLURONIC® F-127, TWEEN® 20, Brij-35, and n-heptyl-β-D-glucopyranoside. The detergent can comprise about 0.1% to about 2% of the binding reagent, filtering reagent, and/or the washing reagent, and/or ranges from about 10 mM up to about 100 mM. The binding reagent, filtering reagent and/or the washing reagent can have a pH ranging from about pH 6.0 to about pH 8.0 (such as from about 6.5 to about 7.5).

The sample supernatant is then removed and the nucleic acid is eluted in an elution buffer such as a Tris/EDTA buffer. The elution buffer can comprise ammonia or an alkali metal hydroxide. In general, the elution buffer has a pH above about 9, above about 10, or above about 11. The elution buffer can further comprise a polyanion, optionally a carrageenan, a carrier nucleic acid, or i-carrageenan and KOH. The eluate may then be processed in the cartridge to detect target genes as described herein. In some embodiments, the eluate is used to reconstitute at least some of the PCR reagents, which are present in the cartridge as lyophilized particles. Particularly, the lyophilized particles can be in the form of beads and comprise primers, probes, a salt, dNTPs, a thermostable polymerase, a reverse transcriptase, or a combination thereof. The lyophilized can be present in the reaction vessel of the cartridge.

As would be appreciated by the skilled artisan, a Ct value is the number of cycles in a quantitative PCR experiment that are required for the fluorescent signal associated with the amplification of a specific target nucleic acid to exceed a predetermined threshold value. As would be appreciated by the skilled artisan, this threshold value can be the background fluorescence levels measured in the experiment.

A biological sample can be any type of biological material isolated from a subject. In some aspects, a biological sample comprises blood. In some aspects, a biological sample can comprise saliva. In some aspects, a biological sample can comprise a nasal swab sample. In some aspects, a biological sample can comprise blood, plasma, serum, urine, breast milk, cerebrospinal fluid, mucus, gastric juice, peritoneal fluid, pleural fluid, saliva, sebum, semen, sweat, tears, vaginal secretion, vomit, endolymph, perilymph or any combination thereof.

In some aspects, a biological sample in a training set can comprise blood. In some aspects, a biological sample in a training set can comprise saliva. In some aspects, a biological sample in a training set can comprise a nasal swab sample. In some aspect, a biological sample in a training set can comprise blood, plasma, serum, urine, breast milk, cerebrospinal fluid, mucus, gastric juice, peritoneal fluid, pleural fluid, saliva, sebum, semen, sweat, tears, vaginal secretion, vomit, endolymph, perilymph or any combination thereof.

Biological samples used in the methods of the present disclosure can be collected from a subject using appropriate methods known in the art, as would be appreciated by the skilled artisan. In some examples, the biological sample can be collected in a Minivette® point of care test (POCT; Sarstedt, Germany). The Minivette® POCT can hold volumes from 10 μL to 200 μL, preferably from 10 μL to 50 μL. and 50 ul volumes. The Minivette® POCT may have neutral, heparin or EDTA preparations.

In some aspects, the biological sample is whole blood collected from a patient. Where a biological sample comprises blood, the blood can be collected from a subject using any blood collection method known in the art, as would be appreciated by the skilled artisan. For example, the whole blood can be collected from the patient by capillary (e.g., from a finger-prick aka finger-stick) or venous puncture blood draw. Accordingly, the present disclosure is used to detect biomarkers of tuberculosis in finger prick volumes (5-50 μL) or greater, of whole blood. In some embodiments, a volume of blood used in the methods disclosed herein can be 1000 μL or less, 500 μL or less, 400 μL or less, 300 μL or less, 200 μL or less, 100 μL or less, or 50 μL or less. The volume of blood is typically about 50 μL or less (e.g. 40 μL or less, 25 μL or less, 15 μL or less, 10 μL or less, or 5 μL or less). In some embodiments, the volume of blood used in the methods is at least 0.5 μL, at least 1 μL, at least 2 μL, at least 5 μL, at least 15 μL, at least 25 μL, or at least 50 μL.

In aspects wherein a biological sample comprises blood, the blood can be collected from a subject using the PAXgene® (QIAGEN/BD, Hombrechtikon, Switzerland) collection method, as would be appreciated by the skilled artisan.

In aspects wherein a biological sample comprises blood, the blood can be collected from a subject using EDTA sample collection tube, as would be appreciated by the skilled artisan.

In some aspects wherein a biological sample comprises blood, the blood can be collected from a subject and subsequently mixed with a stabilization solution. In some examples, the biological sample can comprise whole blood supplemented with anticoagulant agents or with RNA-stabilization buffers. As would be appreciated by the skilled artisan, a non-limiting example of a stabilization solution is RNAlater™ (Thermo Fisher Scientific, US).

In some aspects, biomarkers, including DNA and RNA, can be extracted from biological samples using any method known in the art, including, but not limited to, methods described in U.S. Pat. No. 10,465,182, which is herein incorporated by reference in its entirety.

In aspects wherein the biomarkers to be measured are RNA transcripts, RNA can be extracted from the biological sample using any suitable RNA extraction method known in the art, as would be appreciated by the skilled artisan.

In aspects wherein the biomarkers to be measured are proteins, protein can be extracted from the biological sample using any suitable protein extraction method known in the art, as would be appreciated by the skilled artisan.

In some embodiments, the presence of the biomarkers can be measured in samples collected at one or more times from a subject to monitor treatment for tuberculosis in the subject. In some embodiments, the assay may be used in a subject suspected of respiratory tract infection, e.g., after consultation with their healthcare provider. In some embodiments, the present assay may be used as part of routine and/or preventative healthcare for a subject. In some embodiments, the present assay may be used seasonally as part of routine and/or preventative healthcare for a subject. In some embodiments, the present assay may be used as part of routine and/or preventative healthcare for subjects who are at particular risk from tuberculosis.

In some embodiments, the sample to be tested is obtained from an individual who has one or more symptoms of tuberculosis.

In some embodiments, methods described herein can be used for routine screening of healthy individuals with no risk factors. In some embodiments, methods described herein are used to screen asymptomatic individuals, for example, during routine or preventative care. In some embodiments, methods described herein are used to screen women who are pregnant or who are attempting to become pregnant. In some embodiments the method is used to test patients prior to immunosuppressive therapy.

In some embodiments, the methods described herein can be used to assess the effectiveness of a treatment for tuberculosis infection in a patient.

The methods described herein can be carried out at the same facility where the biological sample was collected from a subject. For example, the method can be a point-of-care method. In other instances, the method can be carried out in a hospital, an urgent care center, an emergency room, a physician's office, a health clinic, or a home. In further instances, the method is a Clinical Laboratory Improvement Amendments (CLIA)-waived test. In some embodiments, information concerning the diagnosis of tuberculosis in the subject is communicated to a medical practitioner. A “medical practitioner,” as used herein, refers to an individual or entity that diagnoses and/or treats patients, such as a hospital, a clinic, a physician's office, a physician, a nurse, or an agent of any of the aforementioned entities and individuals. In some embodiments, the methods are carried out at a laboratory that has received the subject's sample from the medical practitioner or agent of the medical practitioner. The laboratory carries out the detection by any method, including those described herein, and then communicates the results to the medical practitioner. A result is “communicated,” as used herein, when it is provided by any means to the medical practitioner. In some embodiments, such communication may be oral or written, may be by telephone, in person, by e-mail, by mail or other courier, or may be made by directly depositing the information into, e.g., a database accessible by the medical practitioner, including databases not controlled by the medical practitioner. In some embodiments the result of the assay is combined with clinical parameters, data, or information about other risk factors, for example a chest x-ray, to make a diagnosis. In some embodiments, the information is maintained in electronic form. In some embodiments, the information can be stored in a memory or other computer readable medium, such as RAM, ROM, EEPROM, flash memory, computer chips, digital video discs (DVD), compact discs (CDs), hard disk drives (HDD), magnetic tape, etc. The results may also be provided using a web-based application that may be provided to the health care practitioner or to the patient on a smart phone or other mobile device. In some aspects, results may be provided to the patient via a mobile device.

In some embodiments, the method further comprises receiving a communication from the laboratory that indicates the diagnosis of tuberculosis in the sample. A “laboratory,” as used herein, is any facility that detects the target gene in a sample by any method, including the methods described herein, and communicates the result to a medical practitioner. In some embodiments, a laboratory is under the control of a medical practitioner. In some embodiments, a laboratory is not under the control of the medical practitioner.

As used herein, when a method relates to diagnosing tuberculosis infection, the method includes activities in which the steps of the method are carried out, but the result is negative for the presence of tuberculosis infection. That is, detecting, determining, monitoring, and diagnosing tuberculosis infection include instances of carrying out the methods that result in either positive or negative results.

Exemplary Controls

The assays described herein can comprise detecting the biomarkers and at least one endogenous control. The endogenous control can be a sample adequacy control (SAC). In some such embodiments, if none of the biomarkers are detected in a sample, and the SAC is also not detected in the sample, the assay result is considered “invalid” because the sample may have been insufficient. While not intending to be bound by any particular theory, an insufficient sample may be too dilute, contain too little cellular material, contain an assay inhibitor, etc. In some embodiments, the failure to detect an SAC may indicate that the assay reaction failed. In some embodiments, an endogenous control is an RNA (such as an mRNA, tRNA, ribosomal RNA, etc.).

TBP can be used as a positive control biomarker that is indicative of the quality of the sample. Without wishing to be bound by theory, the use of TBP as a positive control means that when TBP expression is detected at a sufficient level, the sample is deemed to be of high enough quality to continue with analysis, and/or, when TBP expression is not detected at a sufficient level, the sample is deemed to be of low quality and further analysis is not performed using that sample. Accordingly, in some aspects, the first predetermined cutoff value in the preceding methods can be a threshold value of measured TBP expression that indicates that TBP is present in the biological sample. As would be appreciated by the skilled artisan, said threshold value can be derived by the user performing the preceding methods based on the experimental conditions being used to measure the expression levels of the recited biomarkers.

An exogenous control (such as an SPC) can be added during performance of an assay, such as with one or more buffers or reagents. In some embodiments, when a GENEXPERT® system is to be used, the SPC is included in the GENEXPERT® cartridge. In some embodiments, an exogenous control (such as an SPC) is an ARMORED® RNA, which is protected by a bacteriophage coat.

The endogenous control and/or an exogenous control may be detected contemporaneously, such as in the same assay, as detection of the biomarkers. In some embodiments, an assay comprises reagents for detecting the biomarkers and an exogenous control simultaneously in the same assay reaction. For example, an assay reaction can comprise a primer set for amplifying each of the biomarkers, and a primer set for amplifying an exogenous control, and labeled probes for detecting the amplification products (such as, for example, TAQMAN® probes).

The level of a target RNA can be normalized to the endogenous control RNA. Normalization may comprise, for example, determination of the difference of the level of the target RNA to the level of the endogenous control RNA. In some such embodiments, the level of the RNAs are represented by a Ct value obtained from quantitative PCR. In some such embodiments, the difference between two measurements is expressed as ΔCt. ΔCt may be calculated as Ct[target RNA]−Ct[endogenous control] or Ct[endogenous control]−Ct[target RNA]. In certain embodiments, ΔCt=Ct[endogenous control]−Ct[biomarker]. In some embodiments, a threshold ΔCt value is set, above or below which a particular diagnosis is indicated. In some such embodiments, the ΔCt threshold is set as the ΔCt value below which 75% of normal samples are correctly characterized. Different thresholds may be applicable to different assays so in some the threshold may be higher, 80%, 90%, 95%, or 97% for example and in some the threshold may be lower, 50%, 60%, or 70% for example. In some such embodiments, a ΔCt value that is higher than the threshold ΔCt value is indicative of a particular disease diagnosis.

A threshold Ct (or a “cutoff Ct”) value for a target RNA, that is indicative of ATB, ITB or STB may be previously determined. In such embodiments, a control sample may not be assayed concurrently with the test sample. In some embodiments, as discussed herein, a ΔCt threshold value is determined, above which TB is indicated or which differentiates ATB, ITB, and STB, has previously been determined.

In some embodiments, linear discriminant analysis (LDA) is used, for example, to combine two or more of the markers into a single combined scale. In some such embodiments, a single threshold value is used for the markers included in the LDA for each of the signatures, e.g. there is a separate threshold value for each set of markers or each signature analysis.

Exemplary RNA Preparation

Target RNA can be prepared by any appropriate method. Total RNA can be isolated by any method, including, but not limited to, the protocols set forth in Wilkinson, M. (1988) Nucl. Acids Res. 16(22):10,933; and Wilkinson, M. (1988) Nucl. Acids Res. 16(22): 10934, or by using commercially-available kits or reagents, such as the TRIzol® reagent (Invitrogen), Total RNA Extraction Kit (iNtRON Biotechnology), Total RNA Purification Kit (Norgen Biotek Corp.), RNAqueous™ (Invitrogen), MagMAX™ (Applied Biosystems), RecoverAll™ (Invitrogen), RNAeasy (Qiagen), etc.

In some embodiments, RNA levels are measured in a sample in which RNA has not first been purified from the cells. In some such embodiments, the cells are subject to a lysis step to release the RNA. Nonlimiting exemplary lysis methods include sonication (for example, for 2-15 seconds, 8-18 μm at 36 kHz); chemical lysis, for example, using a detergent; and various commercially available lysis reagents (such as RNAeasy lysis buffer, Qiagen). In some embodiments, RNA levels are measured in a sample in which RNA has been isolated.

In some embodiments, RNA is modified before a target RNA is detected. In some embodiments, all of the RNA in the sample is modified. In some embodiments, just the particular target RNAs to be analyzed are modified, e.g., in a sequence-specific manner. In some embodiments, RNA is reverse transcribed. In some such embodiments, RNA is reverse transcribed using a reverse transcriptase enzyme such as MMLV, AMV or variants thereof that have been engineered to have features such as reduced RNAse H activity and increased processivity, sensitivity, and thermostability. Nonlimiting exemplary conditions for reverse transcribing RNA using MMLV reverse transcriptase include incubation from 5 to 20 minutes at 40° C. to 50° C.

When a target RNA is reverse transcribed, a DNA complement of the target RNA is formed. In some embodiments, the complement of a target RNA is detected rather than a target RNA itself (or a DNA copy of the RNA itself). Thus, when the methods discussed herein indicate that a target RNA is detected, or the level of a target RNA is determined, such detection or determination may be carried out on a complement of a target RNA instead of, or in addition to, the target RNA itself. In some embodiments, when the complement of a target RNA is detected rather than the target RNA, a polynucleotide for detection is used that is complementary to the complement of the target RNA. In some such embodiments, a polynucleotide for detection comprises at least a portion that is identical in sequence to the target RNA, although it may contain thymidine in place of uridine, and/or comprise other modified nucleotides.

Exemplary Analytical Methods

Any analytical procedure capable of permitting specific detection of a target gene may be used in the methods herein presented. Exemplary nonlimiting analytical procedures include, but are not limited to, nucleic acid amplification methods, PCR methods, isothermal amplification methods, and other analytical detection methods known to those skilled in the art.

In some embodiments, the method of detecting a target gene comprises amplifying the gene and/or a complement thereof. Such amplification can be accomplished by any method. Exemplary methods include, but are not limited to, isothermal amplification, real time RT-PCR, endpoint RT-PCR, and amplification using T7 polymerase from a T7 promoter annealed to a DNA, such as provided by the SenseAmp Plus™ Kit available at Implen, Germany.

When a target gene is amplified, in some embodiments, an amplicon of the target gene is formed. An amplicon may be single stranded or double-stranded. In some embodiments, when an amplicon is single-stranded, the sequence of the amplicon is related to the target gene in either the sense or antisense orientation. In some embodiments, an amplicon of a target gene is detected rather than the target gene itself. Thus, when the methods discussed herein indicate that a target gene is detected, such detection may be carried out on an amplicon of the target gene instead of, or in addition to, the target gene itself. In some embodiments, when the amplicon of the target gene is detected rather than the target gene, a polynucleotide for detection is used that is complementary to the complement of the target gene. In some embodiments, when the amplicon of the target gene is detected rather than the target gene, a polynucleotide for detection is used that is complementary to the target gene. Further, in some embodiments, multiple polynucleotides for detection may be used, and some polynucleotides may be complementary to the target gene and some polynucleotides may be complementary to the complement of the target gene.

In some embodiments, the method of detecting a target gene comprises PCR, as described below. In some embodiments, detecting one or more target genes comprises real-time monitoring of a PCR reaction, which can be accomplished by any method. Such methods include, but are not limited to, the use of TAQMAN®, molecular beacons, or Scorpions probes (i.e., energy transfer (ET) probes, such as FRET probes) and the use of intercalating dyes, such as SYBR green, EvaGreen, thiazole orange, YO-PRO, TO-PRO, etc.

Nonlimiting exemplary conditions for amplifying a cDNA that has been reverse transcribed from the target RNA are as follows. An exemplary cycle comprises an initial denaturation at 90° C. to 100° C. for 20 seconds to 5 minutes, followed by cycling that comprises denaturation at 90° C. to 100° C. for 1 to 10 seconds, followed by annealing and amplification at 60° C. to 75° C. for 10 to 40 seconds. A further exemplary cycle comprises 20 seconds at 94° C., followed by up to 3 cycles of 1 second at 95° C., 35 seconds at 62° C., 20 cycles of 1 second at 95° C., 20 seconds at 62° C., and 14 cycles of 1 second at 95° C., 35 seconds at 62° C. In some embodiments, for the first cycle following the initial denaturation step, the cycle denaturation step is omitted. In some embodiments, Taq polymerase is used for amplification. In some embodiments, the cycle is carried out at least 10 times, at least 15 times, at least 20 times, at least 25 times, at least 30 times, at least 35 times, at least 40 times, or at least 45 times. In some embodiments, Taq is used with a hot start function. In some embodiments, the amplification reaction occurs in a GENEXPERT® cartridge, and amplification of the target genes and an exogenous control occurs in the same reaction. In some embodiments, detection of the target genes occurs in less than 3 hours, less than 2.5 hours, less than 2 hours, less than 1 hour, less than 45 minutes, less than 40 minutes, less than 35 minutes, or less than 30 minutes from initial denaturation through the last extension.

In some embodiments, detection of a target gene comprises forming a complex comprising a polynucleotide that is complementary to a target gene or to a complement thereof, and a nucleic acid selected from the target gene, a DNA amplicon of the target gene, and a complement of the target gene. Thus, in some embodiments, the polynucleotide forms a complex with a target gene. In some embodiments, the polynucleotide forms a complex with a complement of the target RNA, such as a cDNA that has been reverse transcribed from the target RNA. In some embodiments, the polynucleotide forms a complex with a DNA amplicon of the target gene. When a double-stranded DNA amplicon is part of a complex, as used herein, the complex may comprise one or both strands of the DNA amplicon. Thus, in some embodiments, a complex comprises only one strand of the DNA amplicon. In some embodiments, a complex is a triplex and comprises the polynucleotide and both strands of the DNA amplicon. In some embodiments, the complex is formed by hybridization between the polynucleotide and the target gene, complement of the target gene, or DNA amplicon of the target gene. The polynucleotide, in some embodiments, is a primer or probe.

In some embodiments, the complex is detected. In some embodiments, the complex does not have to be associated at the time of detection. That is, in some embodiments, a complex is formed, the complex is then dissociated or destroyed in some manner, and components from the complex are detected. An example of such a system is a TAQMAN® assay. In some embodiments, when the polynucleotide is a primer, detection of the complex may comprise amplification of the target gene, a complement of the target gene, or a DNA amplicon of the target gene.

In some embodiments the analytical method used for detecting at least one target gene in the methods set forth herein includes real-time quantitative PCR. In some embodiments, the analytical method used for detecting at least one target gene includes the use of a TAQMAN® probe. The assay uses energy transfer (“ET”), such as fluorescence resonance energy transfer (“FRET”), to detect and quantitate the synthesized PCR product. Typically, the TAQMAN® probe comprises a fluorescent dye molecule coupled to the 5′-end and a quencher molecule coupled to the 3′-end, such that the dye and the quencher are in close proximity, allowing the quencher to suppress the fluorescence signal of the dye via FRET. When the polymerase replicates the chimeric amplicon template to which the TAQMAN® probe is bound, the 5′-nuclease of the polymerase cleaves the probe, decoupling the dye and the quencher so that the dye signal (such as fluorescence) is detected. Signal (such as fluorescence) increases with each PCR cycle proportionally to the amount of probe that is cleaved.

In some embodiments, a target gene is considered to be detected if any signal is generated from the TAQMAN® probe during the PCR cycling. For example, in some embodiments, if the PCR includes 40 cycles, if a signal is generated at any cycle during the amplification, the target gene is considered to be present and detected. In some embodiments, if no signal is generated by the end of the PCR cycling, the target gene is considered to be absent and not detected.

In some embodiments, quantitation of the results of real-time PCR assays is done by constructing a standard curve from a nucleic acid of known concentration and then extrapolating quantitative information for target genes of unknown concentration. In some embodiments, the nucleic acid used for generating a standard curve is a DNA (for example, an endogenous control, or an exogenous control). In some embodiments, the nucleic acid used for generating a standard curve is a purified double-stranded plasmid DNA or a single-stranded DNA generated in vitro.

In some embodiments, in order for an assay to distinguish ATB, STB or ITB from other diseases, the Ct values for an endogenous control (such as an SAC) and/or an exogenous control (such as an SPC) must be within a previously-determined valid range. That is, in some embodiments, the absence of TB cannot be confirmed unless the controls are detected, indicating that the assay was successful. In some embodiments, the assay includes an exogenous control. Ct values are inversely proportional to the amount of nucleic acid target in a sample.

In some embodiments, a threshold Ct (or a “cutoff Ct”) value for a target gene (including an endogenous control and/or exogenous control), below which the gene is considered to be detected, has previously been determined. In some embodiments, a threshold Ct is determined using substantially the same assay conditions and system (such as a GENEXPERT®) on which the samples will be tested. In some embodiments a ΔCt value is determined

In addition to the TAQMAN® assays, other real-time PCR chemistries useful for detecting and quantitating PCR products in the methods presented herein include, but are not limited to, Molecular Beacons, Scorpions probes and intercalating dyes, such as SYBR Green, EvaGreen, thiazole orange, YO-PRO, TO-PRO, etc., which are discussed below.

In various embodiments, real-time PCR detection is utilized to detect, in a single multiplex reaction, the biomarkers, and optionally an endogenous control, and an exogenous control. In some multiplex embodiments, a plurality of probes, such as TAQMAN® probes, each specific for a different target, is used. In some embodiments, each target gene-specific probe is spectrally distinguishable from the other probes used in the same multiplex reaction. A nonlimiting exemplary seven-color multiplex system is described, e.g., in Lee et al., BioTechniques, 27: 342-349 and a ten-color multiplex system has been described, e.g., in Xie et al. N Engl J Med 2017; 377:1043-1054 and Chakravorty et al. J Clin Microbiol 2016; 55:183-198.

In some embodiments, quantitation of real-time RT PCR products is accomplished using a dye that binds to double-stranded DNA products, such as SYBR Green, EvaGreen, thiazole orange, YO-PRO, TO-PRO, etc. In some embodiments, the assay is the QuantiTect SYBR Green PCR assay from Qiagen. In this assay, total RNA is first isolated from a sample. Total RNA is subsequently poly-adenylated at the 3′-end and reverse transcribed using a universal primer with poly-dT at the 5′-end. In some embodiments, a single reverse transcription reaction is sufficient to assay multiple target RNAs. Real-time RT-PCR is then accomplished using target RNA-specific primers and an miScript Universal Primer, which comprises a poly-dT sequence at the 5′-end. SYBR Green dye binds non-specifically to double-stranded DNA and upon excitation, emits light. In some embodiments, buffer conditions that promote highly-specific annealing of primers to the PCR template (e.g., available in the QuantiTect SYBR Green PCR Kit from Qiagen) can be used to avoid the formation of non-specific DNA duplexes and primer dimers that will bind SYBR Green and negatively affect quantitation. Thus, as PCR product accumulates, the signal from SYBR Green increases, allowing quantitation of specific products.

Real-time PCR is performed using any PCR instrumentation available in the art. Typically, instrumentation used in real-time PCR data collection and analysis comprises a thermal cycler, optics for fluorescence excitation and emission collection, and optionally a computer and data acquisition and analysis software.

In some embodiments, detection and/or quantitation of real-time PCR products is accomplished using a dye that binds to double-stranded DNA products, such as SYBR Green, EvaGreen, thiazole orange, YO-PRO, TO-PRO, etc. In some embodiments, the analytical method used in the methods described herein is a DASL® (DNA-mediated Annealing, Selection, Extension, and Ligation) Assay. In some embodiments, total RNA is isolated from a sample to be analyzed by any method. Total RNA may then be polyadenylated (>18 A residues are added to the 3′-ends of the RNAs in the reaction mixture). The RNA is reverse transcribed using a biotin-labeled DNA primer that comprises from the 5′ to the 3′ end, a sequence that includes a PCR primer site and a poly-dT region that binds to the poly-dA tail of the sample RNA. The resulting biotinylated cDNA transcripts are then hybridized to a solid support via a biotin-streptavidin interaction and contacted with one or more target RNA-specific polynucleotides. The target RNA-specific polynucleotides comprise, from the 5′-end to the 3′-end, a region comprising a PCR primer site, region comprising an address sequence, and a target RNA-specific sequence.

In some DASL® embodiments, the target RNA-specific sequence comprises at least 8, at least 9, at least 10, at least 11, at least 12, at least 13, at least 14, at least 15, at least 16, at least 17, at least 18, at least 19 contiguous nucleotides having a sequence that is the same as, or complementary to, at least 8, at least 9, at least 10, at least 11, at least 12, at least 13, at least 14, at least 15, at least 16, at least 17, at least 18, at least 19 contiguous nucleotides of a target RNA, an endogenous control RNA, or an exogenous control RNA.

After hybridization, the target RNA-specific polynucleotide is extended, and the extended products are then eluted from the immobilized cDNA array. A second PCR reaction using a fluorescently-labeled universal primer generates a fluorescently-labeled DNA comprising the target RNA-specific sequence. The labeled PCR products are then hybridized to a microbead array for detection and quantitation.

In some embodiments, the analytical method used for detecting and quantifying the target genes in the methods described herein is a bead-based flow cytometric assay. See Lu J. et al. (2005) Nature 435:834-838, which is incorporated herein by reference in its entirety. An example of a bead-based flow cytometric assay is the xMAP® technology of Luminex, Inc. See luminexcorp.com. In some embodiments, total RNA is isolated from a sample and is then labeled with biotin. The labeled RNA is then hybridized to target RNA-specific capture probes (e.g., FlexmiR™ products sold by Luminex, Inc. at luminexcorp.com) that are covalently bound to microbeads, each of which is labeled with 2 dyes having different fluorescence intensities. A streptavidin-bound reporter molecule (e.g., streptavidin-phycoerythrin, also known as “SAPE”) is attached to the captured target RNA and the unique signal of each bead is read using flow cytometry. In some embodiments, the RNA sample is first polyadenylated, and is subsequently labeled with a biotinylated 3DNA™ dendrimer (i.e., a multiple-arm DNA with numerous biotin molecules bound thereto), using a bridging polynucleotide that is complementary to the 3′-end of the poly-dA tail of the sample RNA and to the 5′-end of the polynucleotide attached to the biotinylated dendrimer. The streptavidin-bound reporter molecule is then attached to the biotinylated dendrimer before analysis by flow cytometry. In some embodiments, biotin-labeled RNA is first exposed to SAPE, and the RNA/SAPE complex is subsequently exposed to an anti-phycoerythrin antibody attached to a DNA dendrimer, which can be bound to as many as 900 biotin molecules. This allows multiple SAPE molecules to bind to the biotinylated dendrimer through the biotin-streptavidin interaction, thus increasing the signal from the assay.

In some embodiments, the analytical method used for detecting and quantifying the levels of the at least one target gene in the methods described herein is by gel electrophoresis and detection with labeled probes (e.g., probes labeled with a radioactive or chemiluminescent label), such as by northern blotting. In some embodiments, total RNA is isolated from the sample, and then is size-separated by SDS polyacrylamide gel electrophoresis. The separated RNA is then blotted onto a membrane and hybridized to radiolabeled complementary probes. In some embodiments, exemplary probes contain one or more affinity-enhancing nucleotide analogs as discussed below, such as locked nucleic acid (“LNA”) analogs, which contain a bicyclic sugar moiety instead of deoxyribose or ribose sugars. See, e.g., Varallyay, E. et al. (2008) Nature Protocols 3(2):190-196, which is incorporated herein by reference in its entirety.

In some embodiments, detection and quantification of one or more target genes is accomplished using microfluidic devices and single-molecule detection. In some embodiments, target RNAs in a sample of isolated total RNA are hybridized to two probes, one which is complementary to nucleic acids at the 5′-end of the target RNA and the second which is complementary to the 3′-end of the target RNA. Each probe comprises, in some embodiments, one or more affinity-enhancing nucleotide analogs, such as LNA nucleotide analogs and each is labeled with a different fluorescent dye having different fluorescence emission spectra (i.e., detectably different dyes). The sample is then flowed through a microfluidic capillary in which multiple lasers excite the fluorescent probes, such that a unique coincident burst of photons identifies a particular target RNA, and the number of particular unique coincident bursts of photons can be counted to quantify the amount of the target RNA in the sample. In some alternative embodiments, a target RNA-specific probe can be labeled with 3 or more distinct labels selected from, e.g., fluorophores, electron spin labels, etc., and then hybridized to an RNA sample.

Exemplary Automation and Systems

In some embodiments, gene expression is detected using an automated sample handling and/or analysis platform. In some embodiments, commercially available automated analysis platforms are utilized. For example, in some embodiments, the GENEXPERT® system (Cepheid, Sunnyvale, Calif.) is utilized.

The present disclosure is illustrated for use with the GENEXPERT® system. Exemplary sample preparation and analysis methods are described below. However, the present disclosure is not limited to a particular detection method or analysis platform. One of skill in the art recognizes that any number of platforms and methods may be utilized.

The GENEXPERT® utilizes a self-contained, single use cartridge. Sample extraction, amplification, and detection may all carried out within this self-contained “laboratory in a cartridge.” (See e.g., U.S. Pat. Nos. 5,958,349; 6,403,037; 6,440,725; 6,783,736; 6,818,185; each of which is herein incorporated by reference in its entirety.) The cartridge can be a Clinical Laboratory Improvement Amendments (CLIA)-compliant cartridge, operated in compliance with CLIA, is operated by a CLIA-compliant laboratory, or is operated in a CLIA-compliant location.

Components of the cartridge include, but are not limited to, processing chambers containing reagents, filters, and capture technologies useful to extract, purify, and amplify target nucleic acids. A valve enables fluid transfer from chamber to chamber and contain nucleic acids lysis and filtration components. An optical window enables real-time optical detection. A reaction tube enables very rapid thermal cycling.

In some embodiments, the GENEXPERT® system includes a plurality of modules for scalability. Each module includes a plurality of cartridges, along with sample handling and analysis components.

After the sample is added to the cartridge, the sample is contacted with lysis buffer and released nucleic acid (NA) is bound to an NA-binding substrate such as a silica or glass substrate. The sample supernatant is then removed and the NA eluted in an elution buffer such as a Tris/EDTA buffer. The eluate may then be processed in the cartridge to detect target genes as described herein. In some embodiments, the eluate is used to reconstitute at least some of the PCR reagents, which are present in the cartridge as lyophilized particles.

In some embodiments, RT-PCR is used to amplify and analyze the presence of the target genes. In some embodiments, the reverse transcription uses MMLV RT enzyme and an incubation of 5 to 20 minutes at 40° C. to 50° C. In some embodiments, the PCR uses Taq polymerase with hot start function, such as AptaTaq (Roche). In some embodiments, the initial denaturation is at 90° C. to 100° C. for 20 seconds to 5 minutes; the cycling denaturation temperature is 90° C. to 100° C. for 1 to 10 seconds; the cycling anneal and amplification temperature is 60° C. to 75° C. for 10 to 40 seconds; and up to 50 cycles are performed. In some embodiments a different RT may be used. It may be from another organism or may be a natural or engineered variant of an RT enzyme that may be optimized for different temperature incubations.

The present disclosure is not limited to particular primer and/or probe sequences.

Exemplary Data Analysis

In some embodiments, a computer-based analysis program is used to translate the raw data generated by the detection assay into data of predictive value for a clinician. The clinician can access the predictive data using any suitable means. Thus, in some embodiments, the present disclosure provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present disclosure contemplates any method capable of receiving, processing, and transmitting the information to and from laboratories conducting the assays, information provides, medical personal, and subjects. For example, in some embodiments of the present disclosure, a sample (e.g., a blood sample) is obtained from a subject and submitted to a profiling (e.g., clinical lab at a medical facility), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample or sputum sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment for the subject, with or without recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may choose further intervention or counseling based on the results. In some embodiments, the data is used for research use. For example, the data may be used to further optimize the inclusion or elimination of markers as useful indicators of a particular condition or stage of disease or as a companion diagnostic to determine a treatment course of action.

Exemplary Polynucleotides

In some embodiments, polynucleotides are provided. In some embodiments, synthetic polynucleotides are provided. Synthetic polynucleotides, as used herein, refer to polynucleotides that have been synthesized in vitro either chemically or enzymatically. Chemical synthesis of polynucleotides includes, but is not limited to, synthesis using polynucleotide synthesizers, such as OligoPilot (Cytiva), ABI 3900 DNA Synthesizer (Applied Biosystems), and the like. Enzymatic synthesis includes, but is not limited, to producing polynucleotides by enzymatic amplification, e.g., PCR. A polynucleotide may comprise one or more nucleotide analogs (i.e., modified nucleotides) discussed herein.

In various embodiments, a polynucleotide comprises fewer than 500, fewer than 300, fewer than 200, fewer than 150, fewer than 100, fewer than 75, fewer than 50, fewer than 40, or fewer than 30 nucleotides. In various embodiments, a polynucleotide is between 6 and 200, between 8 and 200, between 8 and 150, between 8 and 100, between 8 and 75, between 8 and 50, between 8 and 40, between 8 and 30, between 15 and 100, between 15 and 75, between 15 and 50, between 15 and 40, or between 15 and 30 nucleotides long.

The polynucleotide can be a primer. In some embodiments, the primer is labeled with a detectable moiety. In some embodiments, a primer is not labeled. A primer, as used herein, is a polynucleotide that is capable of selectively hybridizing to a target RNA or to a cDNA reverse transcribed from the target RNA or to an amplicon that has been amplified from a target RNA or a cDNA (collectively referred to as “template”), and, in the presence of the template, a polymerase and suitable buffers and reagents, can be extended to form a primer extension product.

The polynucleotide can be a probe. In some embodiments, the probe is labeled with a detectable moiety. A detectable moiety, as used herein, includes both directly detectable moieties, such as fluorescent dyes, and indirectly detectable moieties, such as members of binding pairs. When the detectable moiety is a member of a binding pair, in some embodiments, the probe can be detectable by incubating the probe with a detectable label bound to the second member of the binding pair. In some embodiments, a probe is not labeled, such as when a probe is a capture probe, e.g., on a microarray or bead. In some embodiments, a probe is not extendable, e.g., by a polymerase. In other embodiments, a probe is extendable.

In some embodiments, the polynucleotide is a FRET probe that in some embodiments is labeled at the 5′-end with a fluorescent dye (donor) and at the 3′-end with a quencher (acceptor), a chemical group that absorbs (i.e., suppresses) fluorescence emission from the dye when the groups are in close proximity (i.e., attached to the same probe). Thus, in some embodiments, the emission spectrum of the dye should overlap considerably with the absorption spectrum of the quencher. In other embodiments, the dye and quencher are not at the ends of the FRET probe.

Exemplary Polynucleotide Modifications

In some embodiments, the methods of detecting at least one target gene described herein employ one or more polynucleotides that have been modified, such as polynucleotides comprising one or more affinity-enhancing nucleotide analogs. Modified polynucleotides useful in the methods described herein include primers for reverse transcription, PCR amplification primers, and probes. In some embodiments, the incorporation of affinity-enhancing nucleotides increases the binding affinity and specificity of a polynucleotide for its target nucleic acid as compared to polynucleotides that contain only deoxyribonucleotides, and allows for the use of shorter polynucleotides or for shorter regions of complementarity between the polynucleotide and the target nucleic acid.

In some embodiments, affinity-enhancing nucleotide analogs include nucleotides comprising one or more base modifications, sugar modifications and/or backbone modifications.

In some embodiments, modified bases for use in affinity-enhancing nucleotide analogs include 5-methylcytosine, isocytosine, pseudoisocytosine, 5-bromouracil, 5-propynyluracil, 6-aminopurine, 2-aminopurine, inosine, diaminopurine, 2-chloro-6-aminopurine, xanthine and hypoxanthine.

In some embodiments, affinity-enhancing nucleotide analogs include nucleotides having modified sugars such as 2′-substituted sugars, such as 2′-O-alkyl-ribose sugars, 2′-amino-deoxyribose sugars, 2′-fluoro-deoxyribose sugars, 2′-fluoro-arabinose sugars, and 2′-O-methoxyethyl-ribose (2′MOE) sugars. In some embodiments, modified sugars are arabinose sugars, or d-arabino-hexitol sugars.

In some embodiments, affinity-enhancing nucleotide analogs include backbone modifications such as the use of peptide nucleic acids (PNA; e.g., an oligomer including nucleobases linked together by an amino acid backbone). Other backbone modifications include phosphorothioate linkages, phosphodiester modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy, and combinations thereof.

In some embodiments, a polynucleotide includes at least one affinity-enhancing nucleotide analog that has a modified base, at least nucleotide (which may be the same nucleotide) that has a modified sugar, and/or at least one internucleotide linkage that is non-naturally occurring.

In some embodiments, an affinity-enhancing nucleotide analog contains a locked nucleic acid (“LNA”) sugar, which is a bicyclic sugar. In some embodiments, a polynucleotide for use in the methods described herein comprises one or more nucleotides having an LNA sugar. In some embodiments, a polynucleotide contains one or more regions consisting of nucleotides with LNA sugars. In other embodiments, a polynucleotide contains nucleotides with LNA sugars interspersed with deoxyribonucleotides. See, e.g., Frieden, M. et al. (2008) Curr. Pharm. Des. 14(11):1138-1142.

Exemplary Primers

In some embodiments, a primer and primer pairs are used. In some embodiments, a primer is at least 85%, at least 90%, at least 95%, or 100% identical to, or at least 85%, at least 90%, at least 95%, or 100% complementary to, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides of the biomarker targets.

In some embodiments, a primer may also comprise portions or regions that are not identical or complementary to the target gene. In some embodiments, a region of a primer that is at least 85%, at least 90%, at least 95%, or 100% identical or complementary to a target gene is contiguous, such that any region of a primer that is not identical or complementary to the target gene does not disrupt the identical or complementary region.

In some embodiments, a primer comprises a portion that is at least 85%, at least 90%, at least 95%, or 100% identical to a region of a target gene. In some such embodiments, a primer that comprises a region that is at least 85%, at least 90%, at least 95%, or 100% identical to a region of the target gene is capable of selectively hybridizing to a cDNA that has been reverse transcribed from the RNA, or to an amplicon that has been produced by amplification of the target gene. In some embodiments, the primer is complementary to a sufficient portion of the cDNA or amplicon such that it selectively hybridizes to the cDNA or amplicon under the conditions of the particular assay being used.

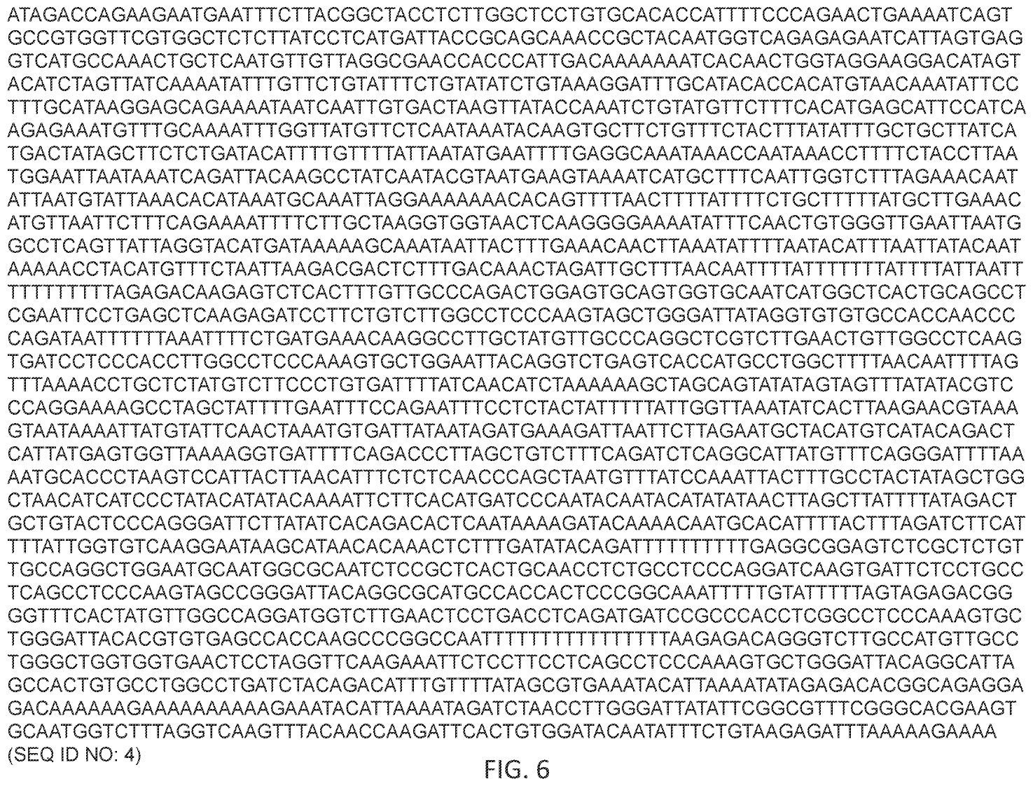

In specific examples, a primer pair for DUSP3 produces an amplicon that is 50 to 500 nucleotides long, 50 to 400 nucleotides long, 50 to 300 nucleotides long, 50 to 200 nucleotides long, 90 to 150 nucleotides long, or 50 to 150 nucleotides long. An example of the nucleotide sequence of DUSP3 is published in the NCBI database under the accession number AC003098. More specifically, a primer pair for DUSP3 can produce an amplicon spanning exons 2 and/or 3 of the nucleotide sequence of DUSP3 published in the NCBI database under the accession number AC003098. In other specific examples, a primer pair for GBP5 produces an amplicon that is 50 to 500 nucleotides long, 50 to 400 nucleotides long, 50 to 300 nucleotides long, 50 to 200 nucleotides long, 90 to 150 nucleotides long, or 50 to 150 nucleotides long. An example of the nucleotide sequence of GBP5 is published in the NCBI database under the accession number AC099063. More specifically, a primer pair for DUSP3 can produce an amplicon spanning exons 9 and/or 10 of the nucleotide sequence of GBP5 published in the NCBI database under the accession number AC099063. In further specific examples, a primer pair for TBP produces an amplicon that is 50 to 500 nucleotides long, 50 to 400 nucleotides long, 50 to 300 nucleotides long, 50 to 200 nucleotides long, 90 to 150 nucleotides long, or 50 to 150 nucleotides long. An example of the nucleotide sequence of TBP is published in the NCBI database under the accession number AL031259. More specifically, a primer pair for TBP can produce an amplicon spanning exons 3 and/or 4 of the nucleotide sequence of TBP published in the NCBI database under the accession number AL031259. In some embodiments, at least one primer of the primer pair for detecting TBP can comprise a sequence that is identical or complementary to at least 10 (at least 12, at least 14, or at least 15) contiguous nucleotides of SEQ ID NO: 4 (FIG. 6). In further more specific examples, a primer pair for TBP can include SEQ ID NO: 1: CCCGAAACGCCGAATATAATCC (forward primer) and SEQ ID NO: 2: CTCCTGTGCACACCATTTTCC (reverse primer).

As used herein, “selectively hybridize” means that a polynucleotide, such as a primer or probe, will hybridize to a particular nucleic acid in a sample with at least 5-fold greater affinity than it will hybridize to another nucleic acid present in the same sample that has a different nucleotide sequence in the hybridizing region. Exemplary hybridization conditions are discussed herein, for example, in the context of a reverse transcription reaction or a PCR amplification reaction. In some embodiments, a polynucleotide will hybridize to a particular nucleic acid in a sample with at least 10-fold greater affinity than it will hybridize to another nucleic acid present in the same sample that has a different nucleotide sequence in the hybridizing region.

In some embodiments, a primer is used to reverse transcribe a target RNA, for example, as discussed herein. In some embodiments, a primer is used to amplify a target RNA or a cDNA reverse transcribed therefrom. Such amplification, in some embodiments, is quantitative PCR, for example, as discussed herein.

In some embodiments, a primer comprises a detectable moiety.

In some embodiments, primer pairs are used. Such primer pairs are designed to amplify a portion of a biomarker gene, or an endogenous control such as a sample adequacy control (SAC), or an exogenous control such as a sample processing control (SPC). In some embodiments, a primer pair is designed to produce an amplicon that is 50 to 1500 nucleotides long, 50 to 1000 nucleotides long, 50 to 750 nucleotides long, 50 to 500 nucleotides long, 50 to 400 nucleotides long, 50 to 300 nucleotides long, 50 to 200 nucleotides long, 50 to 150 nucleotides long, 100 to 300 nucleotides long, 100 to 200 nucleotides long, or 100 to 150 nucleotides long.

Design of primers and probes for amplification of RNA fragments may be performed using DNA Software, Inc.'s Visual OMP (Oligonucleotide Modeling Platform). Visual OMP models, in silico, the folding and hybridization of single-stranded nucleic acids by incorporating all public domain thermodynamic parameters as well as proprietary nearest-neighbor and multi-state thermodynamic parameters for DNA, RNA, PNA, and Inosine. This enables the effective design of primers and probes for complex assays such as microarrays, microfluidics applications and multiplex PCR. In silico experiments simulate secondary structures for targets (optimal and suboptimal), primers (optimal and suboptimal), homodimers, and target and primer heterodimers, given specified conditions. Values for melting temperature (Tm), free energy (AG), percent bound, and concentrations for all species are calculated. Additionally, Visual OMP predicts the binding efficiency between primers and probes with target(s) in a single or multiplex reaction.

Using this software tool, predicted interactions between oligonucleotides and the different targets may be evaluated thermodynamically and unwanted interactions minimized.

Exemplary Probes

In various embodiments, methods of measuring the levels of the biomarkers comprise hybridizing nucleic acids of a sample with a probe.

In some embodiments, the probe comprises a portion that is complementary to a target gene, or an endogenous control such as a sample adequacy control (SAC), or an exogenous control such as a sample processing control (SPC). In some embodiments, the probe comprises a portion that is at least 85%, at least 90%, at least 95%, or 100% identical to a region of the target gene.

In some such embodiments, a probe that is at least 85%, at least 90%, at least 95%, or 100% complementary to a target gene is complementary to a sufficient portion of the target gene such that it selectively hybridizes to the target gene under the conditions of the particular assay being used. In some embodiments, a probe that is complementary to a target gene comprises a region that is at least 85%, at least 90%, at least 95%, or 100% complementary to at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides of the target gene.

A probe that is at least 85%, at least 90%, at least 95%, or 100% complementary to a target gene may also comprise portions or regions that are not complementary to the target gene. In some embodiments, a region of a probe that is at least 85%, at least 90%, at least 95%, or 100% complementary to a target gene is contiguous, such that any region of a probe that is not complementary to the target gene does not disrupt the complementary region.

In some embodiments, the probe comprises a portion that is at least 85%, at least 90%, at least 95%, or 100% identical to a region of the target gene, or an endogenous control such as a sample adequacy control (SAC), or an exogenous control such as a sample processing control (SPC). In some such embodiments, a probe that comprises a region that is at least 85%, at least 90%, at least 95%, or 100% identical to a region of the target gene is capable of selectively hybridizing to a cDNA that has been reverse-transcribed from a target gene or to an amplicon that has been produced by amplification of the target gene. In some embodiments, the probe is at least 85%, at least 90%, at least 95%, or 100% complementary to a sufficient portion of the cDNA or amplicon such that it selectively hybridizes to the cDNA or amplicon under the conditions of the particular assay being used. In some embodiments, a probe that is complementary to a cDNA or amplicon comprises a region that is at least 85%, at least 90%, at least 95%, or 100% complementary to at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides of the cDNA or amplicon. A probe that is at least 85%, at least 90%, at least 95%, or 100% complementary to a cDNA or amplicon may also comprise portions or regions that are not complementary to the cDNA or amplicon. In some embodiments, a region of a probe that is at least 85%, at least 90%, at least 95%, or 100% complementary to a cDNA or amplicon is contiguous, such that any region of a probe that is not complementary to the cDNA or amplicon does not disrupt the complementary region.

In specific examples, a probe for DUSP3 can include an oligonucleotide 10 to 30, 12 to 25, or 16 to 22 nucleotides long and complementary to a region within the amplicon produced from its primer pair described herein. More specifically, a probe for DUSP3 can include an oligonucleotide complementary to a region within exons 3 and/or 4 of the nucleotide sequence of DUSP3 published in the NCBI database under the accession number AC003098. In other specific examples, a probe for GBP5 can include an oligonucleotide 10 to 30, 12 to 25, or 16 to 22 nucleotides long and complementary to a region within the amplicon produced from its primer pair described herein. More specifically, a primer pair for GBP5 can include an oligonucleotide complementary to a region within exons 3 and/or 4 of the nucleotide sequence of GBP5 published in the NCBI database under the accession number AC099063. In further specific examples, a probe for TBP can include an oligonucleotide 10 to 30, 12 to 25, or 16 to 22 nucleotides long and complementary to a region within the amplicon produced from its primer pair described herein. More specifically, a primer pair for TBP can include an oligonucleotide complementary to a region within exons 3 and/or 4 of the nucleotide sequence of TBP published in the NCBI database under the accession number AL031259. In some embodiments, the probe for detecting TBP can comprise a sequence that is identical or complementary to at least 10 (at least 12, at least 14, or at least 15) contiguous nucleotides of SEQ ID NO: 4 (FIG. 6). In further more specific examples, a probe for TBP can include SEQ ID NO: 3: CCACGAACCACGGCACTGATTTT.

In some embodiments, the method of detecting one or more target genes comprises: (a) reverse transcribing a target RNA to produce a cDNA that is complementary to the target RNA; (b) amplifying the cDNA from (a); and (c) detecting the amount of a target RNA using real time RT-PCR and a detection probe (which may be simultaneous with the amplification step (b)).

As described above, in some embodiments, real time RT-PCR detection may be performed using a FRET probe, which includes, but is not limited to, a TAQMAN® probe, a Molecular beacon probe and a Scorpions probe. In some embodiments, the real time RT-PCR detection is performed with a TAQMAN® probe, i.e., a linear probe that typically has a fluorescent dye covalently bound at one end of the DNA and a quencher molecule covalently bound elsewhere, such as at the other end of, the DNA. The FRET probe comprises a sequence that is complementary to a region of the cDNA or amplicon such that, when the FRET probe is hybridized to the cDNA or amplicon, the dye fluorescence is quenched, and when the probe is digested during amplification of the cDNA or amplicon, the dye is released from the probe and produces a fluorescence signal. In some embodiments, the amount of target gene in the sample is proportional to the amount of fluorescence measured during amplification.

The TAQMAN® probe typically comprises a region of contiguous nucleotides having a sequence that is at least 85%, at least 90%, at least 95%, or 100% identical or complementary to a region of a target gene or its complementary cDNA that is reverse transcribed from the target RNA template (i.e., the sequence of the probe region is complementary to or identically present in the target RNA to be detected) such that the probe is selectively hybridizable to a PCR amplicon of a region of the target gene. In some embodiments, the probe comprises a region of at least 6 contiguous nucleotides having a sequence that is fully complementary to or identically present in a region of a cDNA that has been reverse transcribed from a target gene. In some embodiments, the probe comprises a region that is at least 85%, at least 90%, at least 95%, or 100% identical or complementary to at least 8 contiguous nucleotides, at least 10 contiguous nucleotides, at least 12 contiguous nucleotides, at least 14 contiguous nucleotides, or at least 16 contiguous nucleotides having a sequence that is complementary to or identically present in a region of a cDNA reverse transcribed from a target gene to be detected.

In some embodiments, the region of the amplicon that has a sequence that is at least 85%, at least 90%, at least 95%, or 100% complementary to the TAQMAN® probe sequence is at or near the center of the amplicon molecule. In some embodiments, there are independently at least 2 nucleotides, such as at least 3 nucleotides, such as at least 4 nucleotides, such as at least 5 nucleotides of the amplicon at the 5′-end and at the 3′-end of the region of complementarity.

In some embodiments, Molecular Beacons can be used to detect PCR products. Like TAQMAN® probes, Molecular Beacons use FRET to detect a PCR product via a probe having a fluorescent dye and a quencher attached at the ends of the probe. Unlike TAQMAN®® probes, Molecular Beacons remain intact during the PCR cycles. Molecular Beacon probes form a stem-loop structure when free in solution, thereby allowing the dye and quencher to be in close enough proximity to cause fluorescence quenching. When the Molecular Beacon hybridizes to a target, the stem-loop structure is abolished so that the dye and the quencher become separated in space and the dye fluoresces. Molecular Beacons are available, e.g., from Gene Link™ (see genelink.com).

In some embodiments, Scorpion probes can be used as both sequence-specific primers and for PCR product detection. Like Molecular Beacons, Scorpions probes form a stem-loop structure when not hybridized to a target nucleic acid. However, unlike Molecular Beacons, a Scorpions probe achieves both sequence-specific priming and PCR product detection. A fluorescent dye molecule is attached to the 5′-end of the Scorpions probe, and a quencher is attached elsewhere, such as to the 3′-end. The 3′ portion of the probe is complementary to the extension product of the PCR primer, and this complementary portion is linked to the 5′-end of the probe by a non-amplifiable moiety. After the Scorpions primer is extended, the target-specific sequence of the probe binds to its complement within the extended amplicon, thus opening up the stem-loop structure and allowing the dye on the 5′-end to fluoresce and generate a signal. Scorpions probes are available from, e.g., Premier Biosoft International (see premierbiosoft.com).

In some embodiments, labels that can be used on the FRET probes include colorimetric and fluorescent dyes such as Alexa Fluor dyes, BODIPY dyes, such as BODIPY FL; Cascade Blue; Cascade Yellow; coumarin and its derivatives, such as 7-amino-4-methylcoumarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dye™; Marina Blue; Oregon Green; rhodamine dyes, such as rhodamine red, tetramethylrhodamine and rhodamine 6G; Texas Red; fluorescent energy transfer dyes, such as thiazole orange-ethidium heterodimer; and, TOTAB.

Specific examples of dyes include, but are not limited to, those identified above and the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′, 4′,5′,7′-Tetrabromosulfonefluorescein, and TET.

Examples of dye/quencher pairs (i.e., donor/acceptor pairs) include, but are not limited to, fluorescein/tetramethylrhodamine; IAEDANS/fluorescein; EDANS/dabcyl; fluorescein/fluorescein; BODIPY FL/BODIPY FL; fluorescein/QSY 7 or QSY 9 dyes. When the donor and acceptor are the same, FRET may be detected, in some embodiments, by fluorescence depolarization. Certain specific examples of dye/quencher pairs (i.e., donor/acceptor pairs) include, but are not limited to, Alexa Fluor 350/Alexa Fluor488; Alexa Fluor 488/Alexa Fluor 546; Alexa Fluor 488/Alexa Fluor 555; Alexa Fluor 488/Alexa Fluor 568; Alexa Fluor 488/Alexa Fluor 594; Alexa Fluor 488/Alexa Fluor 647; Alexa Fluor 546/Alexa Fluor 568; Alexa Fluor 546/Alexa Fluor 594; Alexa Fluor 546/Alexa Fluor 647; Alexa Fluor 555/Alexa Fluor 594; Alexa Fluor 555/Alexa Fluor 647; Alexa Fluor 568/Alexa Fluor 647; Alexa Fluor 594/Alexa Fluor 647; Alexa Fluor 350/QSY35; Alexa Fluor 350/dabcyl; Alexa Fluor 488/QSY 35; Alexa Fluor 488/dabcyl; Alexa Fluor 488/QSY 7 or QSY 9; Alexa Fluor 555/QSY 7 or QSY9; Alexa Fluor 568/QSY 7 or QSY 9; Alexa Fluor 568/QSY 21; Alexa Fluor 594/QSY 21; and Alexa Fluor 647/QSY 21. In some instances, the same quencher may be used for multiple dyes, for example, a broad spectrum quencher, such as an Iowa Black® quencher (Integrated DNA Technologies, Coralville, Iowa) or a Black Hole Quencher™ (BHQ™; Sigma-Aldrich, St. Louis, Mo.).

In some embodiments, for example, in a multiplex reaction in which two or more moieties (such as amplicons) are detected simultaneously, each probe comprises a detectably different dye such that the dyes may be distinguished when detected simultaneously in the same reaction. One skilled in the art can select a set of detectably different dyes for use in a multiplex reaction.

Specific examples of fluorescently labeled ribonucleotides useful in the preparation of PCR probes for use in some embodiments of the methods described herein are available from Molecular Probes (Invitrogen), and these include, Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Cytiva, such as Cy3-UTP and Cy5-UTP.

Examples of fluorescently labeled deoxyribonucleotides useful in the preparation of PCR probes for use in the methods described herein include Dinitrophenyl (DNP)-1′-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, Alexa Fluor 647-12-OBEA-dCTP. Fluorescently labeled nucleotides are commercially available and can be purchased from, e.g., Thermo Fisher.

In some embodiments, the FRET probe can further comprise other non-natural modifications that reduce primer-dimer amplification in a multiplex polymerase chain reaction (PCR). U.S. Pat. Nos. 9,598,456B2 and 9,598,455B2 describe modified bases that provide enhanced base-pairing affinity in hybridization complexes, the disclosures of which are hereby incorporated by reference.

In some embodiments, dyes and other moieties, such as quenchers, are introduced into polynucleotide used in the methods described herein, such as FRET probes, via modified nucleotides. A “modified nucleotide” refers to a nucleotide that has been chemically modified, but still functions as a nucleotide. In some embodiments, the modified nucleotide has a chemical moiety, such as a dye or quencher, covalently attached, and can be introduced into a polynucleotide, for example, by way of solid phase synthesis of the polynucleotide. In other embodiments, the modified nucleotide includes one or more reactive groups that can react with a dye or quencher before, during, or after incorporation of the modified nucleotide into the nucleic acid. In specific embodiments, the modified nucleotide is an amine-modified nucleotide, i.e., a nucleotide that has been modified to have a reactive amine group. In some embodiments, the modified nucleotide comprises a modified base moiety, such as uridine, adenosine, guanosine, and/or cytosine. In specific embodiments, the amine-modified nucleotide is selected from 5-(3-aminoallyl)-UTP; 8-[(4-amino)butyl]-amino-ATP and 8-[(6-amino)butyl]-amino-ATP; N6-(4-amino)butyl-ATP, N6-(6-amino)butyl-ATP, N4-[2,2-oxy-bis-(ethylamine)]-CTP; N6-(6-Amino)hexyl-ATP; 8-[(6-Amino)hexyl]-amino-ATP; 5-propargylamino-CTP, 5-propargylamino-UTP. In some embodiments, nucleotides with different nucleobase moieties are similarly modified, for example, 5-(3-aminoallyl)-GTP instead of 5-(3-aminoallyl)-UTP. Many amine modified nucleotides are commercially available from, e.g., Applied Biosystems, Sigma, Jena Bioscience and TriLink.

Exemplary detectable moieties also include, but are not limited to, members of binding pairs. In some such embodiments, a first member of a binding pair is linked to a polynucleotide. The second member of the binding pair is linked to a detectable label, such as a fluorescent label. When the polynucleotide linked to the first member of the binding pair is incubated with the second member of the binding pair linked to the detectable label, the first and second members of the binding pair associate and the polynucleotide can be detected. Exemplary binding pairs include, but are not limited to, biotin and streptavidin, antibodies and antigens, etc.

In some embodiments, multiple target genes are detected in a single multiplex reaction. In some such embodiments, each probe that is targeted to a unique amplicon is spectrally distinguishable when released from the probe, in which case each target gene is detected by a unique fluorescence signal. In some embodiments, two or more target genes are detected using the same fluorescent signal, in which case detection of that signal indicates the presence of either of the target genes or both.

One skilled in the art can select a suitable detection method for a selected assay, e.g., a real-time RT-PCR assay. The selected detection method need not be a method described above, and may be any method.

Exemplary Compositions and Kits

In another aspect, compositions are provided. In some embodiments, compositions are provided for use in the methods described herein.

In some embodiments, compositions are provided that comprise at least one target gene-specific primer. The terms “target gene-specific primer” and “target RNA-specific primer” are used interchangeably and encompass primers that have a region of contiguous nucleotides having a sequence that is (i) at least 85%, at least 90%, at least 95%, or 100% identical to a region of a target gene, or (ii) at least 85%, at least 90%, at least 95%, or 100% complementary to the sequence of a region of contiguous nucleotides found in a target gene. In some embodiments, a composition is provided that comprises at least one pair of target gene-specific primers. The term “pair of target gene-specific primers” encompasses pairs of primers that are suitable for amplifying a defined region of a target gene. A pair of target gene-specific primers typically comprises a first primer that comprises a sequence that is at least 85%, at least 90%, at least 95%, or 100% identical to the sequence of a region of a target gene and a second primer that comprises a sequence that is at least 85%, at least 90%, at least 95%, or 100% complementary to a region of a target gene. A pair of primers is typically suitable for amplifying a region of a target gene that is 50 to 1500 nucleotides long, 50 to 1000 nucleotides long, 50 to 750 nucleotides long, 50 to 500 nucleotides long, 50 to 400 nucleotides long, 50 to 300 nucleotides long, 50 to 200 nucleotides long, 50 to 150 nucleotides long, 100 to 300 nucleotides long, 100 to 200 nucleotides long, or 100 to 150 nucleotides long.

In some embodiments, a composition comprises at least one pair of target gene-specific primers. In some embodiments, a composition additionally comprises a pair of target gene-specific primers for amplifying an endogenous control (such as an SAC) and/or one pair of target gene-specific primers for amplifying an exogenous control (such as an SPC). For example, the composition can comprise one or more pair of target gene-specific primers selected from SEQ ID NO: 1, SEQ ID NO: 2, or a combination thereof.

In some embodiments, a composition comprises at least one target gene-specific probe. The terms “target gene-specific probe” and “target RNA-specific probe” are used interchangeably and encompass probes that have a region of contiguous nucleotides having a sequence that is (i) at least 85%, at least 90%, at least 95%, or 100% identical to a region of a target gene, or (ii) at least 85%, at least 90%, at least 95%, or 100% complementary to the sequence of a region of contiguous nucleotides found in a target gene.

In some embodiments, a composition (including a composition described above that comprises one or more pairs of target gene-specific primers) comprises one or more probes for detecting the target genes. In some embodiments, a composition comprises a probe for detecting an endogenous control (such as an SAC) and/or a probe for detecting an exogenous control (such as an SPC). For example, the composition can comprise one or more probes for detecting the target genes including SEQ ID NO: 3.

In some embodiments, a composition is an aqueous composition. In some embodiments, the aqueous composition comprises a buffering component, such as phosphate, tris, HEPES, etc., and/or additional components, as discussed below. In some embodiments, a composition is dry, for example, lyophilized, and suitable for reconstitution by addition of fluid. A dry composition may include one or more buffering components and/or additional components.

In some embodiments, a composition further comprises one or more additional components. Additional components include, but are not limited to, salts, such as NaCl, KCl, and MgCl₂; polymerases, including thermostable polymerases such as Taq; dNTPs; reverse transcriptases, such as MMLV reverse transcriptase; Rnase inhibitors; bovine serum albumin (BSA) and the like; reducing agents, such as β-mercaptoethanol; EDTA and the like; etc. One skilled in the art can select suitable composition components depending on the intended use of the composition.

In some embodiments, compositions are provided that comprise at least one polynucleotide for detecting at least one target gene. In some embodiments, the polynucleotide is used as a primer for a reverse transcriptase reaction. In some embodiments, the polynucleotide is used as a primer for amplification. In some embodiments, the polynucleotide is used as a primer for PCR. In some embodiments, the polynucleotide is used as a probe for detecting at least one target gene. In some embodiments, the polynucleotide is detectably labeled. In some embodiments, the polynucleotide is a FRET probe. In some embodiments, the polynucleotide is a TAQMAN® probe, a Molecular Beacon, or a Scorpions probe.

In some embodiments, a composition comprises at least one FRET probe having a sequence that is at least 85%, at least 90%, at least 95%, or 100% identical, or at least 85%, at least 90%, at least 95%, or 100% complementary, to a region of a target gene. In some embodiments, a FRET probe is labeled with a donor/acceptor pair such that when the probe is digested during the PCR reaction, it produces a unique fluorescence emission that is associated with a specific target gene. In some embodiments, when a composition comprises multiple FRET probes, each probe is labeled with a different donor/acceptor pair such that when the probe is digested during the PCR reaction, each one produces a unique fluorescence emission that is associated with a specific probe sequence and/or target gene. In some embodiments, the sequence of the FRET probe is complementary to a target region of a target gene. In other embodiments, the FRET probe has a sequence that comprises one or more base mismatches when compared to the sequence of the best-aligned target region of a target gene.

In some embodiments, a composition comprises a FRET probe consisting of at least 8, at least 9, at least 10, at least 11, at least 13, at least 14, at least 15, at least 16, at least 17, at least 18, at least 19, at least 20, at least 21, at least 22, at least 23, at least 24, or at least 25 nucleotides, wherein at least a portion of the sequence is at least 85%, at least 90%, at least 95%, or 100% identical, or at least 85%, at least 90%, at least 95%, or 100% complementary, to a region of, a target gene. In some embodiments, at least 8, at least 9, at least 10, at least 11, at least 13, at least 14, at least 15, at least 16, at least 17, at least 18, at least 19, at least 20, at least 21, at least 22, at least 23, at least 24, or at least 25 nucleotides of the FRET probe are identically present in, or complementary to a region of, a target gene. In some embodiments, the FRET probe has a sequence with one, two or three base mismatches when compared to the sequence or complement of the target gene.

In some embodiments, a kit comprises a polynucleotide discussed above. In some embodiments, a kit comprises at least one primer and/or probe discussed above. In some embodiments, a kit comprises at least one polymerase, such as a thermostable polymerase. In some embodiments, a kit comprises dNTPs. In some embodiments, kits for use in the real time RT-PCR methods described herein comprise one or more target gene-specific FRET probes and/or one or more primers for reverse transcription of target RNAs and/or one or more primers for amplification of target genes or cDNAs reverse transcribed therefrom.

In some embodiments, one or more of the primers and/or probes is “linear”. A “linear” primer refers to a polynucleotide that is a single stranded molecule, and typically does not comprise a short region of, for example, at least 3, 4 or 5 contiguous nucleotides, which are complementary to another region within the same polynucleotide such that the primer forms an internal duplex. In some embodiments, the primers for use in reverse transcription comprise a region of at least 4, such as at least 5, such as at least 6, such as at least 7 or more contiguous nucleotides at the 3′-end that has a sequence that is complementary to region of at least 4, such as at least 5, such as at least 6, such as at least 7 or more contiguous nucleotides at the 5′-end of a target gene.

In some embodiments, a kit comprises one or more pairs of linear primers (a “forward primer” and a “reverse primer”) for amplification of a target gene or cDNA reverse transcribed therefrom. Accordingly, in some embodiments, a first primer comprises a region of at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 contiguous nucleotides having a sequence that is at least 85%, at least 90%, at least 95%, or 100% identical to the sequence of a region of at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 contiguous nucleotides at a first location in the target gene. Furthermore, in some embodiments, a second primer comprises a region of at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 contiguous nucleotides having a sequence that is at least 85%, at least 90%, at least 95%, or 100% complementary to the sequence of a region of at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 contiguous nucleotides at a second location in the target gene, such that a PCR reaction using the two primers results in an amplicon extending from the first location of the target gene to the second location of the target gene.

In some embodiments, the kit comprises at least two, at least three, or at least four sets of primers, each of which is for amplification of a different target gene or cDNA reverse transcribed therefrom. In some embodiments, the kit further comprises at least one set of primers for amplifying a control RNA, such as an endogenous control and/or an exogenous control.

In some embodiments, probes and/or primers for use in the compositions described herein comprise deoxyribonucleotides. In some embodiments, probes and/or primers for use in the compositions described herein comprise deoxyribonucleotides and one or more nucleotide analogs, such as LNA analogs or other duplex-stabilizing nucleotide analogs described above. In some embodiments, probes and/or primers for use in the compositions described herein comprise all nucleotide analogs. In some embodiments, the probes and/or primers comprise one or more duplex-stabilizing nucleotide analogs, such as LNA analogs, in the region of complementarity.

In some embodiments, the kits for use in real time RT-PCR methods described herein further comprise reagents for use in the reverse transcription and amplification reactions. In some embodiments, the kits comprise enzymes, such as a reverse transcriptase or a heat stable DNA polymerase, such as Taq polymerase. In some embodiments, the kits further comprise deoxyribonucleotide triphosphates (dNTP) for use in reverse transcription and/or in amplification. In further embodiments, the kits comprise buffers optimized for specific hybridization of the probes and primers.

A kit generally includes a package with one or more containers holding the reagents, as one or more separate compositions or, optionally, as an admixture where the compatibility of the reagents will allow. The kit can also include other material(s) that may be desirable from a user standpoint, such as a buffer(s), a diluent(s), a standard(s), and/or any other material useful in sample processing, washing, or conducting any other step of the assay.

Kits preferably include instructions for carrying out one or more of the methods described herein. Instructions included in kits can be affixed to packaging material or can be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

In some embodiments, the kit can comprise the reagents described above provided in one or more GENEXPERT® cartridge(s). These cartridges permit extraction, amplification, and detection to be carried out within this self-contained “laboratory in a cartridge.” (See e.g., U.S. Pat. Nos. 5,958,349; 6,403,037; 6,440,725; 6,783,736; 6,818,185; 9,873,909; and 10,562,030; each of which is herein incorporated by reference in its entirety.) Reagents for measuring genomic copy number level and detecting a pathogen could be provided in separate cartridges within a kit or these reagents (adapted for multiplex detection) could be provide in a single cartridge.

Any of the kits described here can include, in some embodiments, a receptacle for a blood sample. The receptacle may contain one or more antigens or it may be a Li-heparin tube that does not include antigens.

In some aspects, a kit of the present disclosure can comprise at least about 1 agent, or at least about 2, or at least about 3, or at least about 4, or at least about 5, or at least about 6, or at least about 7, or at least about 8, or at least about 9, or at least about 10 agents specific to detect the expression of at least about one biomarker, or at least about 2, or at least about 3, or at least about 4, or at least about 5, or at least about 6, or at least about 7, or at least about 8, or at least about 9, or at least about 10 biomarkers, including, but not limited to DUSP3, GBP5, and TBP. In some aspects, an agent specific to detect the expression of at least one biomarker can comprise a primer, a pair of primers, a sense and anti-sense primer pair, a polynucleotide that specifically hybridizes to a biomarker or any combination thereof.

The following examples are for illustration purposes only, and are not meant to be limiting in any way.

### EXEMPLARY EMBODIMENTS

Embodiment 1. A method of treating a patient for tuberculosis, comprising:

(a) identifying the patient as having tuberculosis based on the expression levels of GBP5, DUSP3, and TBP biomarkers in a biological sample; and
(b) administering an effective amount of at least one antibiotic to the patient.

Embodiment 2. The method of embodiment 1, wherein the biological sample comprises whole blood, sputum, peripheral blood mononuclear cells, monocytes, or macrophages.

Embodiment 3. The method of any one of embodiments 1-2, wherein the biological sample comprises whole blood, whole blood supplemented with anticoagulant agents or whole blood supplemented with RNA-stabilization buffers.

Embodiment 4. The method of any one of embodiments 1-3, wherein the biological sample is stored at a temperature from 4° C. to 35° C. for up to 24 hours prior to measuring the expression levels of the biomarkers.

Embodiment 5. The method of any one of embodiments 1-4, wherein the biological sample is stored at room temperature up to 35° C. for up to 24 hours prior to measuring the expression levels of the biomarkers.

Embodiment 6. The method of any one of embodiments 1-5, wherein the biomarkers are RNA biomarkers quantified by PCR.

Embodiment 7. The method of embodiment 6, comprising:

contacting the biological sample from the patient with sets of primers that detect GBP5, DUSP3, and TBP biomarkers in the biological sample, wherein the set of primers that detects TBP includes a first primer and a second primer comprising nucleic acid sequences from 12 to 25 nucleotides long;

generating amplicons that are produced by the PCR for GBP5, DUSP3, and TBP; and

contacting the amplicons with at least one probe for each of said biomarkers, wherein each probe comprises nucleic acid sequences from 12 to 25 nucleotides long, and a detectable label.

Embodiment 8. The method of any one of embodiments 1-7, further comprising a step of comparing the level of expression of each biomarker to a reference value for that biomarker or to a control to distinguish active tuberculosis, incipient tuberculosis, and subclinical tuberculosis infected patients.

Embodiment 9. The method of any one of embodiments 1-8, wherein the patient is diagnosed as having active tuberculosis.

Embodiment 10. The method of any one of embodiments 1-9, wherein the patient is administered at least one antibiotic selected from the group consisting of rifampicin, isoniazid, pyrazinamide, ethambutol, rifapentine, ethionamide, moxifloxacin, and streptomycin.

Embodiment 11. The method of embodiment 9 or 10, further comprising administering an effective amount of a corticosteroid to the patient with active tuberculosis.

Embodiment 12. The method of any one of embodiments 1-11, further comprising monitoring the patient's response to treatment by:

(c) comparing expression levels of the biomarkers in the biological sample from step (a) to the expression levels of the biomarkers in a second biological sample, wherein the second biological sample is obtained from the patient at a second time point, or calculating TB scores based on the levels of expression of the biomarkers in the first biological sample and the second biological sample, to determine if the tuberculosis infection in the patient is improving or worsening; and

(d) optionally administering a second treatment regimen to the patient.

Embodiment 13. A method for diagnosing and treating active tuberculosis infection in a patient, the method comprising:

a) contacting a biological sample to be analyzed for the presence of said active tuberculosis infection with primer sets that detect GBP5, DUSP3, and TBP biomarkers, wherein each primer in the primer sets is at least 10 nucleotides in length;

b) generating amplicons of each GBP5, DUSP3, and TBP biomarker;

c) contacting the amplicons with at least one probe for each of said biomarkers, wherein each probe comprises a detectable label;

d) measuring the expression levels of each biomarker and diagnosing the patient as having an active tuberculosis infection based on the expression levels of the biomarkers; and

e) administering an effective amount of at least one antibiotic to the patient diagnosed with active tuberculosis.

Embodiment 14. The method of embodiment 13, wherein the set of primers for detecting TBP includes a first primer and a second primer comprising nucleic acid sequences from 12 to 25 nucleotides long; and wherein the probe for detecting TBP comprises a nucleic acid sequence from 12 to 25 nucleotides long.

Embodiment 15. A method for diagnosing and treating various stages of tuberculosis infection in a patient comprising:

(a) obtaining a first biological sample from the patient;
(b) measuring levels of expression of DUSP3, GBP5, and TBP biomarkers in the first biological sample;
(c) comparing the level of expression of each of the biomarkers to a reference value for that biomarker or to a control;
(d) diagnosing the patient as having active tuberculosis, incipient tuberculosis, or subclinical tuberculosis by analyzing the expression levels of each biomarker in conjunction with respective reference value ranges for each biomarker; and
(e) administering an effective amount of at least one antibiotic to the patient.

Embodiment 16. The method of embodiment 15, further comprising monitoring the patient's response to treatment by

f) measuring levels of expression of GBP5, DUSP3, and TBP biomarkers in a second biological sample from the patient, wherein the second biological sample is obtained from the patient at a second time point;

g) comparing the levels of expression of the biomarkers in the first biological sample to the levels of expression of the biomarkers in the second biological sample, or calculating TB scores based on the levels of expression of the biomarkers in the first biological sample and the second biological sample, to determine if the tuberculosis infection in the patient is improving or worsening; and

h) optionally administering a second treatment regimen to the patient.

Embodiment 17. The method of any one of embodiments 13-16, wherein the biological sample is stored at a temperature from 4° C. to 35° C. for up to 24 hours prior to measuring the expression levels of the biomarkers.

Embodiment 18. The method of any one of embodiments 13-17, wherein the biological sample is stored at room temperature up to 35° C. for 0.5 to 8 hours prior to measuring the expression levels of the biomarkers.

Embodiment 19. The method of any one of embodiments 13-18, wherein the patient is administered at least one antibiotic selected from the group consisting of rifampicin, isoniazid, pyrazinamide, ethambutol, rifapentine, ethionamide, moxifloxacin, and streptomycin.

Embodiment 20. The method of embodiment 19, further comprising administering an effective amount of a corticosteroid to the patient with active tuberculosis.

Embodiment 21. A kit comprising primers and probes for detecting and/or measuring expression levels of GBP5, DUSP3, and TBP biomarkers,

wherein the primers comprise a first PCR primer pair for detecting a GBP5 biomarker, a second PCR pair for detecting a DUSP3 biomarker, and a third PCR primer pair for detecting a TBP biomarker; and

wherein the probes comprise at least one probe for detecting a GBP5 biomarker, at least one probe for detecting a DUSP3 biomarker, and at least one probe for detecting a TBP biomarker, and

wherein each probe comprises a detectable label.

Embodiment 22. The kit of embodiment 21, wherein each probe comprises a fluorescent dye and a quencher molecule.

Embodiment 23. The kit of embodiments 21 or 22, wherein the third PCR primer pair for detecting TBP comprises nucleic acid sequences from 12 to 25 nucleotides long; and wherein the probe for detecting TBP comprises a nucleic acid sequence from 12 to 25 nucleotides long.

Embodiment 24. A method of monitoring a tuberculosis infection in a patient, the method comprising:

a) measuring levels of expression of GBP5, DUSP3, and TBP biomarkers in a first biological sample from the subject, wherein the first biological sample is obtained from the subject at a first time point;

b) measuring levels of expression of GBP5, DUSP3, and TBP biomarkers in a second biological sample from the subject, wherein the second biological sample is obtained from the subject at a second time point; and

c) comparing the levels of expression of the biomarkers in the first biological sample to the levels of expression of the biomarkers in the second biological sample,

wherein increased levels of expression of the GBP5, DUSP3, or TBP biomarkers in the second biological sample compared to the levels of expression of the biomarkers in the first biological sample indicate that the tuberculosis infection in the patient is improving and decreased levels of expression of the GBP5, DUSP3, or TBP biomarkers in the second biological sample compared to the levels of expression of the biomarkers in the first biological sample indicate that the tuberculosis infection in the patient is worsening, or

increased levels of expression of the GBP5, DUSP3, or TBP biomarkers in the second biological sample compared to the levels of expression of the biomarkers in the first biological sample indicate that the tuberculosis infection in the patient is worsening and decreased levels of expression of the GBP5, DUSP3, or TBP biomarkers in the second biological sample compared to the levels of expression of the biomarkers in the first biological sample indicate that the tuberculosis infection in the patient is improving.

Embodiment 25. A method of monitoring a tuberculosis infection in a subject, the method comprising:

a) measuring levels of expression of GBP5, DUSP3, and TBP biomarkers in a first biological sample from the subject, wherein the first biological sample is obtained from the subject at a first time point;

b) measuring levels of expression of GBP5, DUSP3, and TBP biomarkers in a second biological sample from the subject, wherein the second biological sample is obtained from the subject at a second time point; and

c) calculating TB scores based on the levels of expression of the GBP5, DUSP3, and TBP biomarkers in the first biological sample and the second biological sample,

wherein a lower TB score for the second biological sample compared to the TB score for the first biological sample indicates that the tuberculosis infection in the patient is improving, and a higher TB score for the second biological sample compared to the TB score for the first biological sample indicates that the tuberculosis infection in the patient is worsening; or

wherein a lower TB score for the second biological sample compared to the TB score for the first biological sample indicates that the tuberculosis infection in the patient is worsening, and a higher TB score for the second biological sample compared to the TB score for the first biological sample indicates that the tuberculosis infection in the patient is improving.

Embodiment 26. The method of embodiment 24 or 25, wherein the first time point is prior to treating the patient for tuberculosis and the second time point is after treating the patient with at least one antibiotic for treating tuberculosis.

Embodiment 27. The method of embodiment 24 or 25, wherein the first and second time points are after treating the patient with at least one antibiotic for treating tuberculosis.

Embodiment 28. A method for distinguishing active tuberculosis from not infected, latent tuberculosis and other pulmonary conditions or infectious diseases in a patient, the method comprising:

a) obtaining a biological sample from the patient;

b) measuring levels of expression of GBP5, DUSP3, and TBP biomarkers; and

c) analyzing the levels of expression of the GBP5, DUSP3, and TBP biomarkers in conjunction with respective reference value ranges for said biomarkers,

wherein similarity of the levels of expression of the GBP5, DUSP3, and TBP biomarkers to reference value ranges for a subject with active tuberculosis indicate that the patient has active tuberculosis, and

wherein similarity of the levels of expression of the GBP5, DUSP3, and TBP biomarkers to reference value ranges for a subject not-infected or with latent tuberculosis and other pulmonary conditions or infectious diseases indicate that the patient is not infected, has latent tuberculosis or other pulmonary conditions or infectious diseases.

Embodiment 29. A method for diagnosing tuberculosis in a patient, the method comprising:

a) measuring levels of expression of DUSP3, GBP5, and TBP biomarkers in a biological sample from the patient;
b) determining a score based on the levels of expression of the DUSP3, GBP5 and TBP biomarkers, wherein the score is calculated using the formula:

$$Score = \frac{( GBP 5 + DUSP 3 )}{2} - TBP$$

wherein GBP5 is the level of expression of the GBP5 biomarker measured in step (a), DUSP3 is the level of expression of the DUSP3 biomarker measured in step (a) and TBP is the level of expression of the TBP biomarker measured in step (a); and

c) identifying that the patient has tuberculosis or does not have tuberculosis based on the score.

Embodiment 30. The method of embodiment 29, further comprising administering to a patient identified as having tuberculosis an effective amount of at least one tuberculosis treatment, wherein the at least one tuberculosis treatment comprises at least one antibiotic, at least one corticosteroid or any combination thereof.

Embodiment 31. The method of embodiment 29, wherein the at least one antibiotic is selected from the group consisting of rifampicin, isoniazid, pyrazinamide, ethambutol, rifapentine, ethionamide, moxifloxacin, and streptomycin

Embodiment 32. The method of any one of embodiments 29-31, wherein step (c) comprises comparing the score to a predetermined cutoff value.

Embodiment 33. The method of embodiment 32, wherein:

i) the patient is identified as having tuberculosis when the score is greater than or equal to the predetermined cutoff value and the patient is identified as not having tuberculosis when the score is less than the predetermined cutoff value; or

ii) the patient is identified as having tuberculosis when the score is less than or equal to the predetermined cutoff value and the patient is identified as not having tuberculosis when the score is greater than the predetermined cutoff value.

Embodiment 34. The method of embodiment 32 or 33, wherein the predetermined cutoff value distinguishes between active tuberculosis, incipient tuberculosis, and subclinical tuberculosis infected patients.

Embodiment 35. The method of one of embodiments 32-34, wherein the predetermined cutoff value has a specificity of at least 85%, or at least 90%, or at least 95%, or at least 99.5%.

Embodiment 36. The method of one of embodiments 32-35, wherein the predetermined cutoff value has a sensitivity of at least 85%, or at least 90%, or at least 95%, or at least 99.5%.

Embodiment 37. The method of one of embodiments 32-36, wherein the predetermined cutoff value has a positive predictive value of at least 85%, or at least 90%, or at least 95%, or at least 99.5%.

Embodiment 38. The method of one of embodiments 32-37, wherein the predetermined cutoff value has a negative predictive value of at least 85%, or at least 90%, or at least 95%, or at least 99.5%.

Embodiment 39. The method of any one of embodiments 29-38, wherein the tuberculosis is active tuberculosis, incipient tuberculosis, and subclinical tuberculosis.

Embodiment 40. The method of any one of embodiments 29-39, wherein the biological sample comprises whole blood, sputum, peripheral blood mononuclear cells, monocytes, or macrophages.

Embodiment 41. The method of any one of embodiments 29-40, wherein the biological sample comprises whole blood, whole blood supplemented with anticoagulant agents or whole blood supplemented with RNA-stabilization buffers.

Embodiment 42. The method of any one of embodiments 29-41, wherein the biological sample is stored at a temperature from 4° C. to 35° C. for up to 24 hours prior to measuring the expression levels of the biomarkers.

Embodiment 43. The method of any one of embodiments 29-42, wherein the biological sample is stored at room temperature up to 35° C. for up to 24 hours prior to measuring the expression levels of the biomarkers.

Embodiment 44. The method of any one of embodiments 29-43, wherein the biomarkers are RNA biomarkers quantified by PCR.

Embodiment 45. The method of embodiment 44, comprising:

contacting the biological sample from the patient with sets of primers that detect GBP5, DUSP3, and TBP biomarkers in the biological sample, wherein the set of primers that detects TBP includes a first primer and a second primer comprising nucleic acid sequences from 12 to 25 nucleotides long;

generating amplicons that are produced by the PCR for GBP5, DUSP3, and TBP; and

contacting the amplicons with at least one probe for each of said biomarkers, wherein each probe comprises nucleic acid sequences from 12 to 25 nucleotides long, and a detectable label.

Embodiment 46. A cartridge for distinguishing active tuberculosis from not infected, latent tuberculosis and other pulmonary conditions or infectious diseases, or risk of progressing to active tuberculosis in a patient, the cartridge comprising:

a plurality of processing chambers in fluidic communication, and

a nucleic acid-binding substrate for binding nucleic acid in fluidic communication with the processing chambers,

wherein the processing chambers comprise reagents for lysing cells from a sample, amplification and detection of nucleic acid from the sample, and a composition comprising sets of primers for detecting GBP5, DUSP3, and TBP biomarkers.

Embodiment 47. The cartridge of embodiment 46, wherein the plurality of processing chambers comprises
a lysis chamber in fluidic communication with the nucleic acid-binding substrate, wherein the lysis chamber comprises one or more reagents for lysing cells, and
a reaction tube in fluidic communication with the lysis chamber and configured for amplification of nucleic acid and detection of amplification products.

Embodiment 48. The cartridge of embodiment 46 or 47, wherein the reagents for lysing cells comprise a chaotropic agent, a chelating agent, a buffer, and a detergent.

Embodiment 49. The cartridge of embodiment 48, wherein the chaotropic agent is selected from guanidinium thiocyanate, guanidinium hydrochloride, alkali perchlorate, alkali iodide, urea, formamide, or a combination thereof.

Embodiment 50. The cartridge of any one of embodiments 46-49, wherein
a) the sets of primers for detecting TBP is selected from:
   i) a forward and at least one reverse primer for detecting a sequence of the TBP gene; or
   ii) a forward primer comprising a region of at least 15 contiguous nucleotides having a sequence that is at least 85% identical to at least 15 contiguous nucleotides of the TBP gene at exons 3 and/or 4, and at least one reverse primer comprising a region of at least 15 contiguous nucleotides having a sequence that is at least 85% identical to at least 15 contiguous nucleotides of the TBP gene at exons 3 and/or 4, or
   iii) a forward primer comprising a region of at least 15 contiguous nucleotides having a sequence that is at least 85% identical to at least 15 contiguous nucleotides of SEQ ID NO: 1 and/or SEQ ID NO: 4, and at least one reverse primer comprising a region of at least 15 contiguous nucleotides having a sequence that is at least 85% identical to at least 15 contiguous nucleotides of SEQ ID NO: 2 and/or SEQ ID NO: 4,
b) the sets of primers for detecting GBP5 is selected from:
   i) a forward and at least one reverse primer for detecting a sequence of the GBP5 gene; or
   ii) a forward primer comprising a region of at least 15 contiguous nucleotides having a sequence that is at least 85% identical to at least 15 contiguous nucleotides of the GBP5 gene at exons 9 and/or 10, and at least one reverse primer comprising a region of at least 15 contiguous nucleotides having a sequence that is at least 85% identical to at least 15 contiguous nucleotides of the GBP5 gene at exons 9 and/or 10, and
c) the sets of primers for detecting DUSP3 is selected from:
   i) a forward and at least one reverse primer for detecting a sequence of the DUSP3 gene; or
   ii) a forward primer comprising a region of at least 15 contiguous nucleotides having a sequence that is at least 85% identical to at least 15 contiguous nucleotides of the DUSP3 gene at exons 2 and/or 3, and at least one reverse primer comprising a region of at least 15 contiguous nucleotides having a sequence that is at least 85% identical to at least 15 contiguous nucleotides of the DUSP3 gene at exons 2 and/or 3.

Embodiment 51. A method of identifying if a patient has tuberculosis or does not have tuberculosis, the method comprising:

a) measuring levels of expression of DUSP3, GBP5, and TBP biomarkers in a biological sample from the patient;

b) identifying that the patient has tuberculosis or does not have tuberculosis based on the expression levels measured in step (a).

Embodiment 52. The method of embodiment 51, wherein a patient that is identified as having tuberculosis is a patient that has one of active tuberculosis, subclinical tuberculosis or incipient tuberculosis.

Embodiment 53. The method of embodiment 51 or embodiment 52, wherein a patient that is identified as not having tuberculosis is a patient that has no tuberculosis infection or has latent tuberculosis.

Embodiment 54. The method of any one of embodiments 51-53, wherein identifying that the patient has tuberculosis or does not have tuberculosis based on the expression levels measured in step (a) comprises comparing the level of expression of each biomarker to a respective reference value for that biomarker or to a control to identify that the patient has tuberculosis or does not have tuberculosis.

Embodiment 55. The method of any one of embodiments 51-54, wherein identifying that the patient has tuberculosis or does not have tuberculosis based on the expression levels measured in step (a) comprises:

b₁) determining a score using the levels of expression measured in step (a);

b₂) identifying that the patient has tuberculosis or does not have tuberculosis based on the score.

Embodiment 56. The method of any one of embodiments 51-55, wherein identifying that the patient has tuberculosis or does not have tuberculosis based on the score comprises:

i) comparing the score to a predetermined cutoff value; and

ii) determining that the patient has tuberculosis or does not have tuberculosis based on the relationship between the score and the predetermined cutoff value.

Embodiment 57. The method of any one of embodiments 51-56, wherein determining that the patient has tuberculosis or does not have tuberculosis based on the relationship between the score and the predetermined cutoff value comprises determining that the patient has tuberculosis when the score is greater than or equal to the predetermined cutoff value and determining that the patient does not have tuberculosis when the score is less than the predetermined cutoff value.

Embodiment 58. The method of any one of embodiments 51-57, wherein determining that the patient has tuberculosis or does not have tuberculosis based on the relationship between the score and the predetermined cutoff value comprises determining that the patient has tuberculosis when the score is less than or equal to the predetermined cutoff value and determining that the patient does not have tuberculosis when the score is greater than the predetermined cutoff value.

Embodiment 59. The method of any one of embodiments 51-58, wherein the score is calculated using the formula:

$$Score = \frac{( GBP 5 + DUSP 3 )}{2} - TBP$$

wherein GBP5 is the level of expression of the GBP5 biomarker measured in step (a), DUSP3 is the level of expression of the DUSP3 biomarker measured in step (a) and TBP is the level of expression of the TBP biomarker measured in step (a).

Embodiment 60. The method of any one of embodiments 51-59, wherein the score is calculated using a formula derived from the analysis of the levels of expression of the DUSP3, GBP5, and TBP biomarkers in a plurality of biological samples obtained from a plurality of subject, wherein: i) at least one subject in the plurality of subjects has tuberculosis; and ii) at least one subject in the plurality of subjects does not have tuberculosis.

Embodiment 61. The method of any one of embodiments 51-60, wherein the at least one subject in the plurality of subjects that has tuberculosis has one of active tuberculosis, subclinical tuberculosis or incipient tuberculosis.

Embodiment 62. The method of any one of embodiments 51-61, wherein the at least one subject in the plurality of subjects that does not have tuberculosis has no tuberculosis infection or has latent tuberculosis.

Embodiment 63. The method of any one of embodiments 51-62, wherein when the patient is identified as having tuberculosis, the method further comprises identifying that the patient has active tuberculosis, subclinical tuberculosis or incipient tuberculosis based on the expression levels measured in step (a).

Embodiment 64. The method of any one of embodiments 51-63, wherein identifying that the patient has active tuberculosis, subclinical tuberculosis, or incipient tuberculosis based on the expression levels measured in step (a) comprises comparing the level of expression of each biomarker to one or more respective reference values for that biomarker or to one or more controls to identify that the patient has active tuberculosis, subclinical tuberculosis, or incipient tuberculosis.

Embodiment 65. The method of any one of embodiments 51-64, wherein identifying that the patient has active tuberculosis, subclinical tuberculosis, or incipient tuberculosis based on the expression levels measured in step (a) comprises:

b₁) determining a score using the levels of expression measured in step (a);

b₂) identifying that the patient has active tuberculosis, subclinical tuberculosis, or incipient tuberculosis based on the score.

Embodiment 66. The method of any one of embodiments 51-65, identifying that the patient has active tuberculosis, subclinical tuberculosis, or incipient tuberculosis based on the score comprises:

i) comparing the score to one or more predetermined cutoff values; and

ii) determining that the patient has active tuberculosis, subclinical tuberculosis or incipient tuberculosis based on the relationship between the score and the one or more predetermined cutoff values.

Embodiment 67. The method of any one of embodiments 51-66, wherein the score is calculated using a formula derived from the analysis of the levels of expression of the DUSP3, GBP5, and TBP biomarkers in a plurality of biological samples obtained from a plurality of subjects.

Embodiment 68. The method of any one of embodiments 51-67, wherein:

i) at least one subject in the plurality of subjects has active tuberculosis;

ii) at least one subject in the plurality of subjects has subclinical tuberculosis; and

iii) at least one subject in the plurality of subject has incipient tuberculosis.

Embodiment 69. The method of any one of embodiments 51-68, wherein when the patient is identified as not having tuberculosis, the method further comprises identifying that the patient has no tuberculosis infection or has latent tuberculosis based on the expression levels measured in step (a).

Embodiment 70. The method of any one of embodiments 51-69, wherein identifying that the patient has no tuberculosis infection or has latent tuberculosis based on the expression levels measured in step (a) comprises comparing the level of expression of each biomarker to one or more respective reference values for that biomarker or to one or more controls to identify that the patient has no tuberculosis infection or has latent tuberculosis.

Embodiment 71. The method of any one of embodiments 51-70, wherein identifying that the patient has no tuberculosis infection or has latent tuberculosis based on the expression levels measured in step (a) comprises:

b₁) determining a score using the levels of expression measured in step (a);

b₂) identifying that the patient has no tuberculosis infection or has latent tuberculosis based on the score.

Embodiment 72. The method of any one of embodiments 51-71, wherein identifying that the patient has no tuberculosis infection or has latent tuberculosis based on the score comprises:

i) comparing the score to one or more predetermined cutoff values; and

ii) determining that the patient has no tuberculosis infection or has latent tuberculosis based on the relationship between the score and the one or more predetermined cutoff values.

Embodiment 73. The method of any one of embodiments 51-72, wherein the score is calculated using a formula derived from the analysis of the levels of expression of the DUSP3, GBP5, and TBP biomarkers in a plurality of biological samples obtained from a plurality of subjects, wherein:

i) at least one subject in the plurality of subjects has no tuberculosis infection;

ii) at least one subject in the plurality of subjects has latent tuberculosis.

Embodiment 74. A method of identifying if a patient has tuberculosis, is at high risk of having tuberculosis, is at low risk of having tuberculosis, or does not have tuberculosis, the method comprising:

a) measuring levels of expression of DUSP3, GBP5, and TBP biomarkers in a biological sample from the patient;

b) identifying that the patient:
i) has tuberculosis;
ii) is at high risk of having tuberculosis;
iii) is at low risk of having tuberculosis; or
iv) does not have tuberculosis

based on the expression levels measured in step (a).

Embodiment 75. The method of any one of embodiments 51-74, wherein a patient that is identified as having tuberculosis is a patient that has one of active tuberculosis, subclinical tuberculosis or incipient tuberculosis.

Embodiment 76. The method of any one of embodiments 51-75, wherein a patient that is identified as being at high risk of having tuberculosis is a patient that has a high risk of having one of active tuberculosis, subclinical tuberculosis or incipient tuberculosis.

Embodiment 77. The method of any one of embodiments 51-76, wherein a patient that is identified of being at low risk of having tuberculosis is a patient that has a low risk of having one of active tuberculosis, subclinical tuberculosis or incipient tuberculosis.

Embodiment 78. The method of any one of embodiments 51-77, wherein a patient that is identified as not having tuberculosis is a patient that has no tuberculosis infection or has latent tuberculosis.

Embodiment 79. The method of any one of embodiments 51-79, wherein identifying that the patient has tuberculosis, is at high risk of having tuberculosis, is at low risk of having tuberculosis, or does not have tuberculosis based on the expression levels measured in step (a) comprises comparing the level of expression of each biomarker to one or more respective reference values for that biomarker or to a control.

Embodiment 80. The method of any one of embodiments 51-79, wherein identifying that the patient has tuberculosis, is at high risk of having tuberculosis, is at low risk of having tuberculosis, or does not have tuberculosis based on the expression levels measured in step (a) comprises:

b₁) determining a score using the levels of expression measured in step (a);

b₂) identifying that the patient has tuberculosis, is at high risk of having tuberculosis, is at low risk of having tuberculosis, or does not have tuberculosis based on the score.

Embodiment 81. The method of any one of embodiments 51-80, wherein identifying that the patient has tuberculosis, is at high risk of having tuberculosis, is at low risk of having tuberculosis, or does not have tuberculosis based on the score comprises:

i) comparing the score to a first, second and third predetermined cutoff value; and

ii) determining that the patient:
a) has tuberculosis when the score is greater than or equal to the third predetermined cutoff value;
b) is at high risk of having tuberculosis when the score is lead than the third predetermined cutoff value and greater than or equal to the second predetermined cutoff value;
c) is at low risk of having tuberculosis when the score is less than the second predetermined cutoff value and greater than or equal to the first predetermined cutoff value; or
d) does not have tuberculosis when the score is less than the first predetermined cutoff value.

Embodiment 82. The method of any one of embodiments 51-81, wherein the third predetermined cutoff value has a specificity of at least about 98% and a sensitivity of at least about 45%.

Embodiment 83. The method of any one of embodiments 51-82, wherein the second predetermined cutoff value has a specificity of at least about 70% and a sensitivity of at least about 90%.

Embodiment 84. The method of any one of embodiments 51-83, wherein the first predetermined cutoff value has a specificity of at least about 99% or a sensitivity of at least about 30%.

Embodiment 85. The method of any one of embodiments 51-84, further comprising administering at least one tuberculosis treatment to the patient when the patient is identified as having tuberculosis or is identified as having a high risk of having tuberculosis.

Embodiment 86. A method of monitoring the response of a patient having tuberculosis to a tuberculosis treatment, the method comprising:

a) measuring levels of expression of DUSP3, GBP5, and TBP biomarkers in a first biological sample from the patient obtained at a first time point;

b) measuring levels of expression of DUSP3, GBP5, and TBP biomarkers in a second biological sample from the patient obtained at a second time point; and

c) determining whether the patient is responding to the tuberculosis treatment by comparing the levels of expression from step (a) and the levels of expression from step (b).

Embodiment 87. The method of any one of embodiments 51-86, wherein the first time point is prior to the administration of the tuberculosis treatment to the patient and the second time point is after the administration of at least one amount of the tuberculosis treatment to the patient.

Embodiment 88. The method of any one of embodiments 51-87, wherein the patient has been administered at least one amount of the tuberculosis treatment prior to the first time point and the second time point is after the administration of at least one additional amount of the tuberculosis treatment.

Embodiment 89. The method of any one of embodiments 51-88, wherein the patient has active tuberculosis, subclinical tuberculosis, or incipient tuberculosis.

Embodiment 90. The method of any one of embodiments 51-89, wherein determining whether the patient is responding to the tuberculosis treatment by comparing the levels of expression from step (a) and the levels of expression from step (b) in step (c) comprises:

i) determining a first score using the levels of expression measured in step (a);

ii) determining a second score using the levels of expression measured in step (b);

iii) comparing the first score and the second score; and

iv) determining if the subject is responding to the treatment or not responding to the treatment based on the relationship between the first score and the second score.

Embodiment 91. The method of any one of embodiments 51-90, wherein the score is calculated using a formula derived from the analysis of the levels of expression of the DUSP3, GBP5, and TBP biomarkers in a plurality of biological samples obtained from a plurality of subjects, wherein:

i) at least one subject in the plurality of subjects responded to the tuberculosis treatment; and

ii) at least one subject in the plurality of subjects did not respond to the tuberculosis treatment.

Embodiment 92. A method of identifying if a patient has tuberculosis, a risk of having tuberculosis, or does not have tuberculosis, the method comprising:
a) measuring levels of expression of DUSP3, GBP5, and TBP biomarkers in a biological sample from the patient;
b) identifying the patient as having i) tuberculosis, ii) high risk for having tuberculosis, iii) low risk for having tuberculosis, or iv) no tuberculosis infection based on the expression levels measured in step (a).

Embodiment 93. The method of any one of the preceding embodiments, wherein identifying that the patient has tuberculosis, a risk of having tuberculosis, or does not have tuberculosis based on the expression levels measured in step (a) comprises comparing the level of expression of each biomarker to a respective reference value for that biomarker or to a control to identify that the patient has tuberculosis, high risk for having tuberculosis, low risk for having tuberculosis, or does not have tuberculosis.

Embodiment 94. The method of any one of the preceding embodiments, wherein identifying that the patient has tuberculosis, a risk of having tuberculosis, or does not have tuberculosis based on the expression levels measured in step (a) comprises:
b₁) determining a score using the levels of expression measured in step (a);
b₂) identifying that the patient has tuberculosis, high risk for having tuberculosis, low risk for having tuberculosis, or does not have tuberculosis based on the score.

Embodiment 95. The method of any one of the preceding embodiments, wherein the score is calculated using the formula:

$$Score = \frac{( GBP 5 + DUSP 3 )}{2} - TBP$$

wherein GBP5 is the level of expression of the GBP5 biomarker measured in step (a), DUSP3 is the level of expression of the DUSP3 biomarker measured in step (a) and TBP is the level of expression of the TBP biomarker measured in step (a).

Embodiment 96. The method of any one of the preceding embodiments, wherein the score is calculated using a formula derived from the analysis of the levels of expression of the DUSP3, GBP5, and TBP biomarkers in a plurality of biological samples obtained from a plurality of subject, preferably wherein:

i) at least one subject in the plurality of subjects has tuberculosis; and

ii) at least one subject in the plurality of subjects does not have tuberculosis, preferably:
wherein the at least one subject in the plurality of subjects that has tuberculosis has one of active tuberculosis, subclinical tuberculosis or incipient tuberculosis; and/or
at least one subject in the plurality of subjects that does not have tuberculosis has no tuberculosis infection or has latent tuberculosis.

Embodiment 97. The method of any one of the preceding embodiments, wherein when the patient is identified as having tuberculosis or high risk of having tuberculosis, the method further comprises identifying that the patient has or has a high risk of having active tuberculosis, subclinical tuberculosis or incipient tuberculosis based on the expression levels measured in step (a).

Embodiment 98. The method of any one of the preceding embodiments, wherein identifying that the patient has active tuberculosis, subclinical tuberculosis, or incipient tuberculosis based on the expression levels measured in step (a) comprises comparing the level of expression of each biomarker to one or more respective reference values for that biomarker or to one or more controls to identify that the patient has active tuberculosis, subclinical tuberculosis, or incipient tuberculosis.

Embodiment 99. The method of any one of the preceding embodiments, wherein identifying that the patient has active tuberculosis, subclinical tuberculosis, or incipient tuberculosis based on the expression levels measured in step (a) comprises:
b₁) determining a score using the levels of expression measured in step (a);
b₂) identifying that the patient has active tuberculosis, subclinical tuberculosis, or incipient tuberculosis based on the score.

Embodiment 100. The method of any one of the preceding embodiments, wherein the score is calculated using a formula derived from the analysis of the levels of expression of the DUSP3, GBP5, and TBP biomarkers in a plurality of biological samples obtained from a plurality of subjects, preferably wherein:

i) at least one subject in the plurality of subjects has active tuberculosis;

ii) at least one subject in the plurality of subjects has subclinical tuberculosis; and

iii) at least one subject in the plurality of subject has incipient tuberculosis.

Embodiment 101. A method of identifying if a patient has active tuberculosis, is at high risk of having active tuberculosis, is at low risk of having active tuberculosis, or does not have tuberculosis, the method comprising:
a) measuring levels of expression of DUSP3, GBP5, and TBP biomarkers in a biological sample from the patient;
b) identifying that the patient:
   i) has active tuberculosis;
   ii) is at high risk of having active tuberculosis;
   iii) is at low risk of having active tuberculosis; or
   iv) does not have tuberculosis
based on the expression levels measured in step (a).

Embodiment 102. The method of any one of the preceding embodiments, wherein a patient that is identified of being at low risk of having active tuberculosis is a patient that has subclinical tuberculosis, incipient tuberculosis, or latent tuberculosis.

Embodiment 103. The method of any one of the preceding embodiments, wherein a patient that is identified as not having tuberculosis is a patient that has no tuberculosis infection or has latent tuberculosis.

Embodiment 104. The method of any one of the preceding embodiments, wherein identifying that the patient has active tuberculosis, is at high risk of having active tuberculosis, is at low risk of having active tuberculosis, or does not have tuberculosis based on the expression levels measured in step (a) comprises:
b₁) determining a score using the levels of expression measured in step (a);
b₂) identifying that the patient has tuberculosis, is at high risk of having tuberculosis, is at low risk of having tuberculosis, or does not have tuberculosis based on the score.

Embodiment 105. The method of any one of the preceding embodiments, wherein the score is calculated using the formula:

$$Score = \frac{( GBP 5 + DUSP 3 )}{2} - TBP$$

wherein GBP5 is the level of expression of the GBP5 biomarker measured in step (a), DUSP3 is the level of expression of the DUSP3 biomarker measured in step (a) and TBP is the level of expression of the TBP biomarker measured in step (a).

Embodiment 106. The method of any one of the preceding embodiments, wherein the score is calculated using a formula derived from the analysis of the levels of expression of the DUSP3, GBP5, and TBP biomarkers in a plurality of biological samples obtained from a plurality of subject, preferably wherein:

i) at least one subject in the plurality of subjects has active or probable tuberculosis; and

ii) at least one subject in the plurality of subjects does not have tuberculosis or has latent tuberculosis.

Embodiment 107. The method of any one of the preceding embodiments, wherein identifying that the patient has active tuberculosis, is at high risk of having active tuberculosis, is at low risk of having active tuberculosis, or does not have tuberculosis based on the score comprises:
i) comparing the score to a first, second and third predetermined cutoff value; and
ii) determining that the patient:
   a) has active tuberculosis when the score is greater than or equal to the third predetermined (diagnostic) cutoff value;
   b) is at high risk of having active tuberculosis when the score is lower than the third predetermined cutoff value and greater than or equal to the second predetermined (triage) cutoff value;
   c) is at low risk of having active tuberculosis when the score is less than the second predetermined cutoff value and greater than or equal to the first predetermined (rule-out) cutoff value; or
   d) does not have tuberculosis when the score is less than the first predetermined cutoff value.

Embodiment 108. The method of any one of the preceding embodiments, wherein

i) the third predetermined cutoff value has a specificity of at least about 98% and a sensitivity of at least about 45%;

ii) the second predetermined cutoff value has a specificity of at least about 70% and a sensitivity of at least about 90%; and/or

iii) the first predetermined cutoff value has a specificity of at least about 99% or a sensitivity of at least about 30%.

Embodiment 109. The method of any one of the preceding embodiments, further comprising administering at least one tuberculosis treatment to the patient when the patient is identified as having active tuberculosis or is identified as having a high risk of having tuberculosis.

Embodiment 110. The method of any one of the preceding embodiments, further comprising monitoring responses to tuberculosis treatment and/or monitoring disease progression when the patient is identified as having active tuberculosis or is identified as having a high risk of having tuberculosis.

Embodiment 111. A method of monitoring the response of a patient having tuberculosis to a tuberculosis treatment, the method comprising:
a) measuring levels of expression of DUSP3, GBP5, and TBP biomarkers in a first biological sample from the patient obtained at a first time point;
b) measuring levels of expression of DUSP3, GBP5, and TBP biomarkers in a second biological sample from the patient obtained at a second time point; and
c) determining whether the patient is responding to the tuberculosis treatment by comparing the levels of expression from step (a) and the levels of expression from step (b).

Embodiment 112. The method of any one of the preceding embodiments, wherein:

(i) the first time point is prior to the administration of the tuberculosis treatment to the patient and the second time point is after the administration of at least one amount of the tuberculosis treatment to the patient; or

(ii) the patient has been administered at least one amount of the tuberculosis treatment prior to the first time point and the second time point is after the administration of at least one additional amount of the tuberculosis treatment.

Embodiment 113. The method of any one of the preceding embodiments, wherein the patient has active tuberculosis, high risk of having active tuberculosis, low risk of having active tuberculosis, subclinical tuberculosis, or incipient tuberculosis.

Embodiment 114. The method of any one of the preceding embodiments, wherein determining whether the patient is responding to the tuberculosis treatment by comparing the levels of expression from step (a) and the levels of expression from step (b) in step (c) comprises:

i) determining a first score using the levels of expression measured in step (a);

ii) determining a second score using the levels of expression measured in step (b);

iii) comparing the first score and the second score; and

iv) determining if the subject is responding to the treatment or not responding to the treatment based on the relationship between the first score and the second score.

Embodiment 115. The method of any one of the preceding embodiments, wherein the first score and the second score is calculated using the formula:

$$Score = \frac{( GBP 5 + DUSP 3 )}{2} - TBP$$

wherein GBP5 is the level of expression of the GBP5 biomarker measured in step (a), DUSP3 is the level of expression of the DUSP3 biomarker measured in step (a) and TBP is the level of expression of the TBP biomarker measured in step (a).

Embodiment 116. The method of any one of the preceding embodiments, wherein the subject is identified as responding to the treatment when the second score is less than the first score and the subject is identified as not responding to the treatment when the second score is equal to or greater than the first score.

Embodiment 117. The method of any one of the preceding embodiments, wherein the subject is identified as responding to the treatment when the second score is less than or equal to the first score and the subject is identified as not responding to the treatment when the second score is greater than the first score.

Embodiment 118. The method of any one of the preceding embodiments, wherein the score is calculated using a formula derived from the analysis of the levels of expression of the DUSP3, GBP5, and TBP biomarkers in a plurality of biological samples obtained from a plurality of subjects, wherein:

i) at least one subject in the plurality of subjects responded to the tuberculosis treatment; and

ii) at least one subject in the plurality of subjects did not respond to the tuberculosis treatment.

Embodiment 119. The method of any one of the preceding embodiments, wherein the at least one tuberculosis treatment comprises at least one antibiotic, at least one corticosteroid or any combination thereof, preferably wherein the at least one antibiotic is selected from the group consisting of rifampicin, isoniazid, pyrazinamide, ethambutol, rifapentine, ethionamide, moxifloxacin, and streptomycin.

Embodiment 120. The method of any one of the preceding embodiments, wherein the biological sample comprises:

i) whole blood, sputum, peripheral blood mononuclear cells, monocytes, or macrophages.

ii) whole blood, whole blood supplemented with anticoagulant agents or whole blood supplemented with RNA-stabilization buffers.

Embodiment 121. The method of any one of the preceding embodiments, wherein the biological sample is stored:

i) at a temperature from 4° C. to 35° C. for up to 24 hours prior to measuring the expression levels of the biomarkers;

ii) at room temperature up to 35° C. for up to 24 hours prior to measuring the expression levels of the biomarkers; or

iii) at room temperature up to 35° C. for 0.5 to 8 hours prior to measuring the expression levels of the biomarkers.

Embodiment 122. The method of any one of the preceding embodiments, wherein the biomarkers are RNA biomarkers quantified by PCR, preferably wherein measuring levels of expression of DUSP3, GBP5, and TBP biomarkers in a biological sample from the patient comprises:
contacting the biological sample from the patient with sets of primers that detect GBP5, DUSP3, and TBP biomarkers in the biological sample, wherein each primer in the primer sets is at least 10 nucleotides in length;
generating amplicons that are produced by the PCR for GBP5, DUSP3, and TBP; and contacting the amplicons with at least one probe for each of said biomarkers, wherein each probe comprises and a detectable label.

Embodiment 123. The method of any one of the preceding embodiments, wherein:

i) each probe comprises nucleic acid sequences from 12 to 25 nucleotides long; and/or

ii) the set of primers that detects TBP includes a first primer and a second primer comprising nucleic acid sequences from 12 to 25 nucleotides long.

Embodiment 124. The method of any one of the preceding embodiments, wherein the predetermined cutoff value:
i) has a specificity of at least 85%, or at least 90%, or at least 95%, or at least 99.5%.
ii) has a sensitivity of at least 85%, or at least 90%, or at least 95%, or at least 99.5%.
iii) has a positive predictive value of at least 85%, or at least 90%, or at least 95%, or at least 99.5%; and/or
iv) has a negative predictive value of at least 85%, or at least 90%, or at least 95%, or at least 99.5%.

Embodiment 125. The method of any one of the preceding embodiments, wherein the at least one tuberculosis treatment comprises at least one antibiotic, at least one corticosteroid or any combination thereof.

Embodiment 126. The method of any one of the preceding embodiments, wherein the at least one antibiotic is selected from the group consisting of rifampicin, isoniazid, pyrazinamide, ethambutol, rifapentine, ethionamide, moxifloxacin, and streptomycin.

Embodiment 127. The method of any one of the preceding embodiments, wherein the biological sample comprises whole blood, sputum, peripheral blood mononuclear cells, monocytes, or macrophages.

Embodiment 128. The method of any one of the preceding embodiments, wherein the biological sample comprises whole blood, whole blood supplemented with anticoagulant agents or whole blood supplemented with RNA-stabilization buffers.

Embodiment 129. The method of any one of the preceding embodiments, wherein the biological sample is stored at a temperature from 4° C. to 35° C. for up to 24 hours prior to measuring the expression levels of the biomarkers.

Embodiment 130. The method of any one of the preceding embodiments, wherein the biological sample is stored at room temperature up to 35° C. for up to 24 hours prior to measuring the expression levels of the biomarkers.

Embodiment 131. The method of any one of the preceding embodiments, wherein the biomarkers are RNA biomarkers quantified by PCR.

Embodiment 132. The method of any one of the preceding embodiments, wherein measuring levels of expression of DUSP3, GBP5, and TBP biomarkers in a biological sample from the patient comprises:

contacting the biological sample from the patient with sets of primers that detect GBP5, DUSP3, and TBP biomarkers in the biological sample, wherein each primer in the primer sets is at least 10 nucleotides in length;

generating amplicons that are produced by the PCR for GBP5, DUSP3, and TBP; and

contacting the amplicons with at least one probe for each of said biomarkers, wherein each probe comprises and a detectable label.

Embodiment 133. The method of any one of the preceding embodiments, wherein each probe comprises nucleic acid sequences from 12 to 25 nucleotides long.

Embodiment 134. The method of any one of the preceding embodiments, wherein the set of primers that detects TBP includes a first primer and a second primer comprising nucleic acid sequences from 12 to 25 nucleotides long

Embodiment 135. The method of any one of the preceding embodiments, wherein the biological sample is stored at a temperature from 4° C. to 35° C. for up to 24 hours prior to measuring the expression levels of the biomarkers.

Embodiment 136. The method of any one of the preceding embodiments, wherein the biological sample is stored at room temperature up to 35° C. for 0.5 to 8 hours prior to measuring the expression levels of the biomarkers.

Embodiment 137. The method of any one of the preceding embodiments, wherein the predetermined cutoff value has a specificity of at least 85%, or at least 90%, or at least 95%, or at least 99.5%.

Embodiment 138. The method of any one of the preceding embodiments, wherein the predetermined cutoff value has a sensitivity of at least 85%, or at least 90%, or at least 95%, or at least 99.5%.

Embodiment 139. The method of any one of the preceding embodiments, wherein the predetermined cutoff value has a positive predictive value of at least 85%, or at least 90%, or at least 95%, or at least 99.5%.

Embodiment 140. The method of any one of the preceding embodiments, wherein the predetermined cutoff value has a negative predictive value of at least 85%, or at least 90%, or at least 95%, or at least 99.5%.

Embodiment 141. A cartridge for identifying if a patient has tuberculosis, a risk of having tuberculosis, or does not have tuberculosis, the cartridge comprising:

a plurality of processing chambers in fluidic communication, and

a nucleic acid-binding substrate for binding nucleic acid in fluidic communication with the processing chambers,

wherein the processing chambers comprise reagents for lysing cells from a sample, amplification and detection of nucleic acid from the sample, and a composition comprising sets of primers for detecting GBP5, DUSP3, and TBP biomarkers.

Embodiment 142. The cartridge of any one of the preceding embodiments, wherein the plurality of processing chambers comprises
a lysis chamber in fluidic communication with the nucleic acid-binding substrate, wherein the lysis chamber comprises one or more reagents for lysing cells, and
a reaction tube in fluidic communication with the lysis chamber and configured for amplification of nucleic acid and detection of amplification products.

Embodiment 143. The cartridge of any one of the preceding embodiments, wherein the reagents for lysing cells comprise a chaotropic agent, a chelating agent, a buffer, and a detergent.

Embodiment 144. The cartridge of any one of the preceding embodiments, wherein the chaotropic agent is selected from guanidinium thiocyanate, guanidinium hydrochloride, alkali perchlorate, alkali iodide, urea, formamide, or a combination thereof.

Embodiment 146. The cartridge of any one of the preceding embodiments, wherein
a) the sets of primers for detecting TBP is selected from:
   i) a forward and at least one reverse primer for detecting a sequence of the TBP gene; or
   ii) a forward primer comprising a region of at least 15 contiguous nucleotides having a sequence that is at least 85% identical to at least 15 contiguous nucleotides of the TBP gene at exons 3 and/or 4, and at least one reverse primer comprising a region of at least 15 contiguous nucleotides having a sequence that is at least 85% identical to at least 15 contiguous nucleotides of the TBP gene at exons 3 and/or 4, or
   iii) a forward primer comprising a region of at least 15 contiguous nucleotides having a sequence that is at least 85% identical to at least 15 contiguous nucleotides of SEQ ID NO: 1 and/or SEQ ID NO: 4, and at least one reverse primer comprising a region of at least 15 contiguous nucleotides having a sequence that is at least 85% identical to at least 15 contiguous nucleotides of SEQ ID NO: 2 and/or SEQ ID NO: 4,
b) the sets of primers for detecting GBP5 is selected from:
   i) a forward and at least one reverse primer for detecting a sequence of the GBP5 gene; or
   ii) a forward primer comprising a region of at least 15 contiguous nucleotides having a sequence that is at least 85% identical to at least 15 contiguous nucleotides of the GBP5 gene at exons 9 and/or 10, and at least one reverse primer comprising a region of at least 15 contiguous nucleotides having a sequence that is at least 85% identical to at least 15 contiguous nucleotides of the GBP5 gene at exons 9 and/or 10, and
c) the sets of primers for detecting DUSP3 is selected from:
   i) a forward and at least one reverse primer for detecting a sequence of the DUSP3 gene; or
   ii) a forward primer comprising a region of at least 15 contiguous nucleotides having a sequence that is at least 85% identical to at least 15 contiguous nucleotides of the DUSP3 gene at exons 2 and/or 3, and at least one reverse primer comprising a region of at least 15 contiguous nucleotides having a sequence that is at least 85% identical to at least 15 contiguous nucleotides of the DUSP3 gene at exons 2 and/or 3.

### EXAMPLES

### Example 1: Identification of TBP as a Stably Expressed Gene in Patients with Latent and Active Tuberculosis

To identify genes with low mRNA expression variability, a set of mRNAs with a potential for low variation of expression in whole blood samples was first identified. Illumina probe IDs were obtained for each candidate gene, resulting in a total of 80 corresponding IDs. A GEO dataset GSE19491 (Berry et al 2010) with blood gene expression profiles from patients with active and latent tuberculosis was obtained, and expression data from the set of Illumina probe IDs were extracted. Expression variation was then calculated using the R-script Normfinder (Andersen et al 2004) (Table 1).

TABLE 1
Expression variation determined by Normfinder.

| | | | | | Average |
|---|---|---|---|---|---|
| Genes | | GroupDif | GroupSD | Stability | expression |
| KLF2 | ILMN_1735930 | 0.04 | 0.5 | 0.07 | 6953 |
| UBE2D2 | ILMN_1725644 | 0.14 | 0.51 | 0.1 | 55 |
| EEF1A1 | ILMN_1810810 | 0.1 | 0.74 | 0.12 | 21240 |
| TBP | ILMN_1697117 | 0.25 | 0.56 | 0.13 | 183 |
| SIRT5 | ILMN_1799598 | 0.16 | 0.87 | 0.15 | 25 |
| HPRT1 | ILMN_1736940 | 0.25 | 0.72 | 0.17 | 149 |
| YWHAZ | ILMN_1801928 | 0.4 | 0.34 | 0.17 | 4986 |
| UBC | ILMN_2038773 | 0.34 | 0.58 | 0.18 | 17022 |
| B2M | ILMN_2148459 | 0.41 | 0.57 | 0.2 | 10163 |
| GAPDH | ILMN_1343295 | 0.37 | 0.71 | 0.21 | 1392 |
| ACTB | ILMN_2038777 | 0.41 | 0.6 | 0.21 | 12160 |
| FAM48A | ILMN_1669555 | 0.51 | 0.47 | 0.23 | 202 |
| TRAP1 | ILMN_1699737 | 0.37 | 0.91 | 0.23 | 67 |
| DECR1 | ILMN_1720838 | 0.59 | 0.39 | 0.25 | 958 |
| RPLP0 | ILMN_1709880 | 0.53 | 0.72 | 0.27 | 5664 |
| RAB8B | ILMN_2173004 | 0.59 | 0.65 | 0.27 | 1574 |
| CDC37 | ILMN_1668369 | 0.64 | 0.65 | 0.29 | 2719 |
| DUSP3 | ILMN_1797522 | 2.01 | 1.12 | 0.8 | 774 |
| GBP5 | ILMN_2114568 | 7.57 | 3.31 | 2.7 | 1472 |

Amongst the examined Illumina IDs, the ID corresponding to KLF2 was found to show the lowest variation, whereas the IDs corresponding to GBP5 and DUSP3 were amongst the IDs with highest variation. As shown in the results below, expression variability of KLF2 fluctuates with time and changes in temperature. TBP was found to be amongst the IDs with lowest variation and was selected to go into the XPERT prototype design since 1) TBP displayed expression levels that were more similar to DUSP3 and GBP5 than other genes with low variability e.g. UBE2D2 and EEF1A1, and 2) TBP was a more suitable candidate for PCR optimization relative to e.g. YWHAZ where probe design was limited by the existence of highly similar sequences on multiple chromosomes. The prototype design included primer pairs and probes for TBP (e.g., SEQ ID NO: 1, SEQ ID NO: 2, and SEQ ID NO: 3), primer pairs and probes for GBP5, primer pairs and probes for DUSP3, and optionally primer pairs and probes for KLF2.

### Example 2: TBP Improves Signature Stability in EDTA Blood Compared to KLF2

Whole venous EDTA blood was sampled from donors (n=3-4). The samples were held at different temperatures (4, RT and 35° C.) for 0, 0.5, 1, 2, 4, 8 and 24 h before analysis of 6 replicates per timepoint and temperature with XPERT TB Host Response prototype on a GENEXPERT® instrument. Two scores were calculated, one according to the equation (GBP5+DUSP3)/2−KLF2 (“delta TB-score” used interchangeably with “TB-score”) and one according to (GBP5+DUSP3)/2−TBP (“delta TBP-score” used interchangeably with “TBP-score”). The average of the 6 replicates per time point and temperature was then normalized to the first time point (t0). The data shows that the TBP-score is more stable with time than the TB-score at RT (FIG. 1A) and at 35° C. (FIG. 2A). The TBP-score is stable down to 2 uL WB (data not shown).

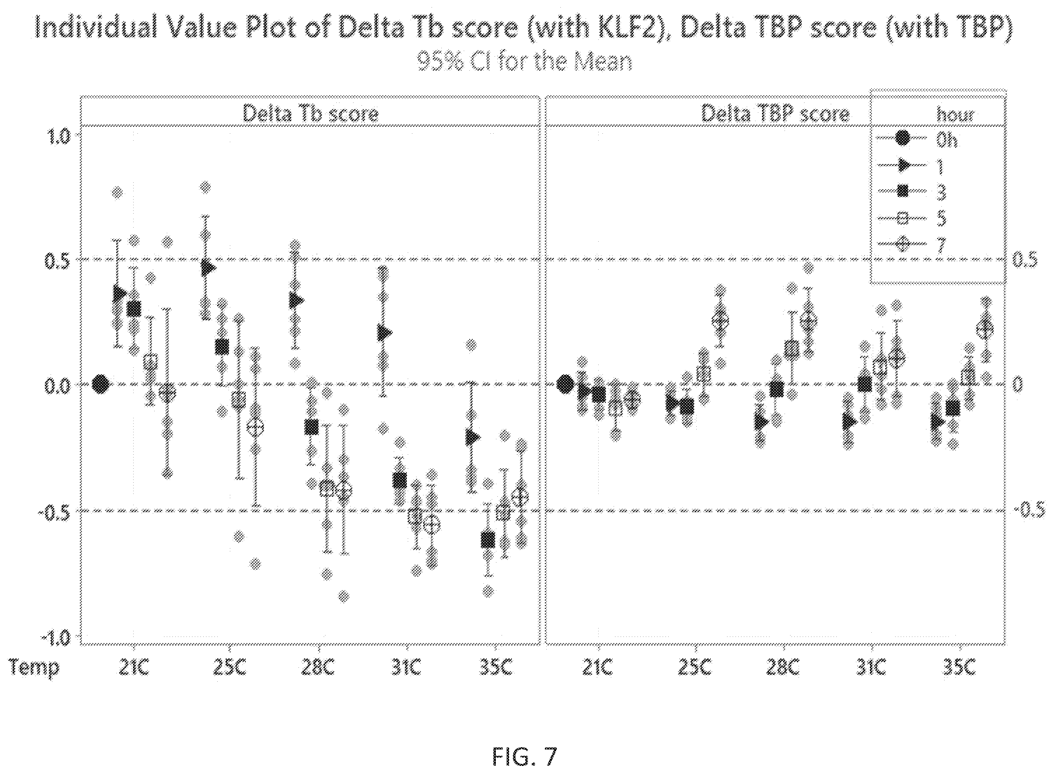

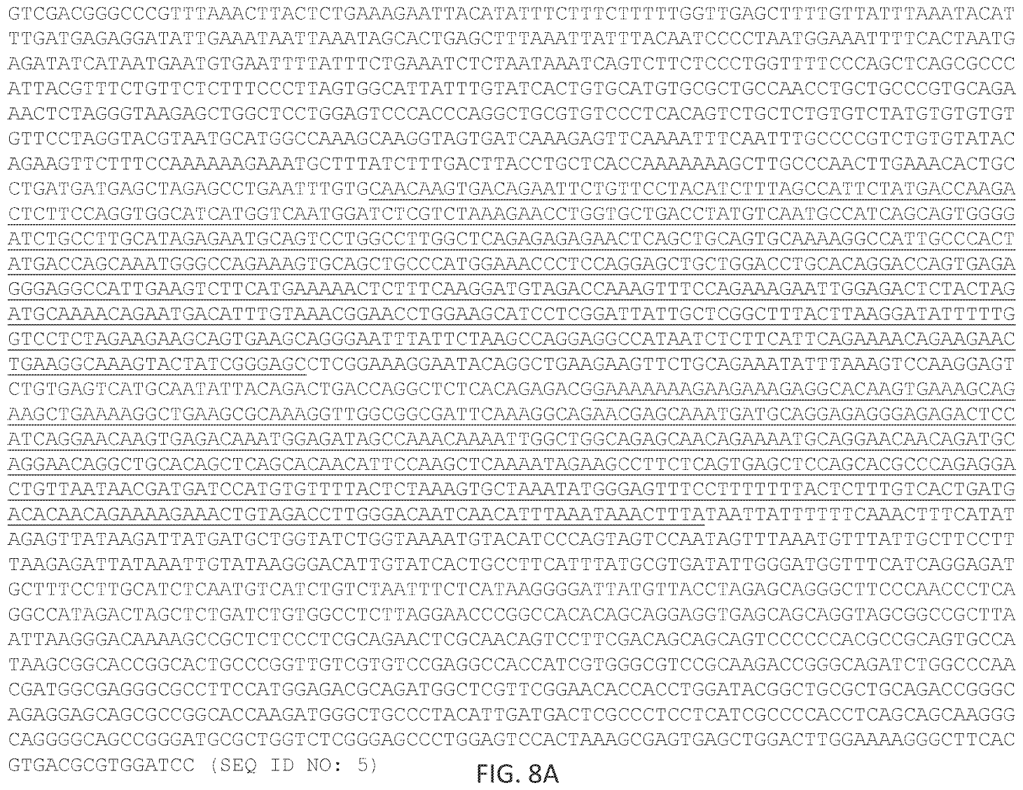

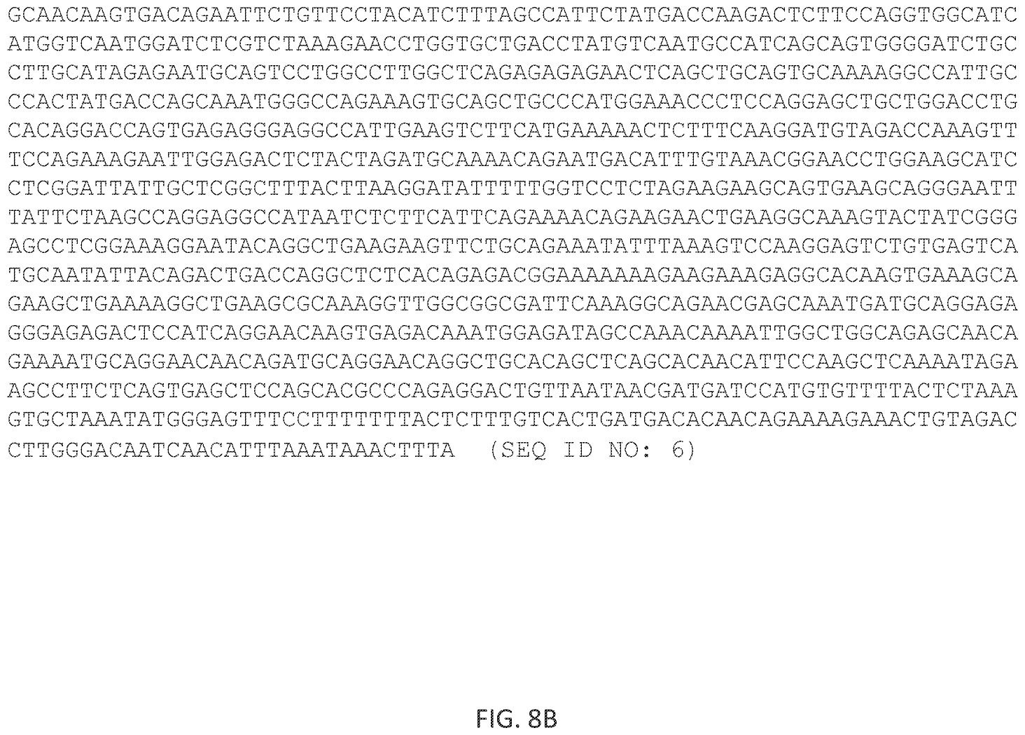

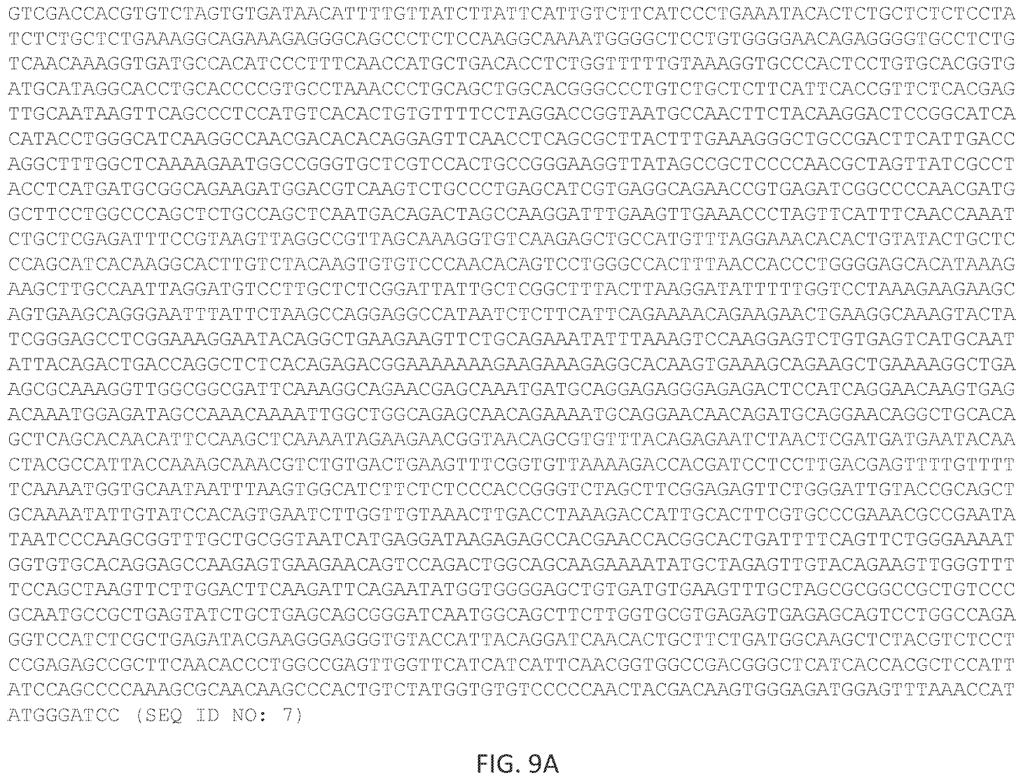

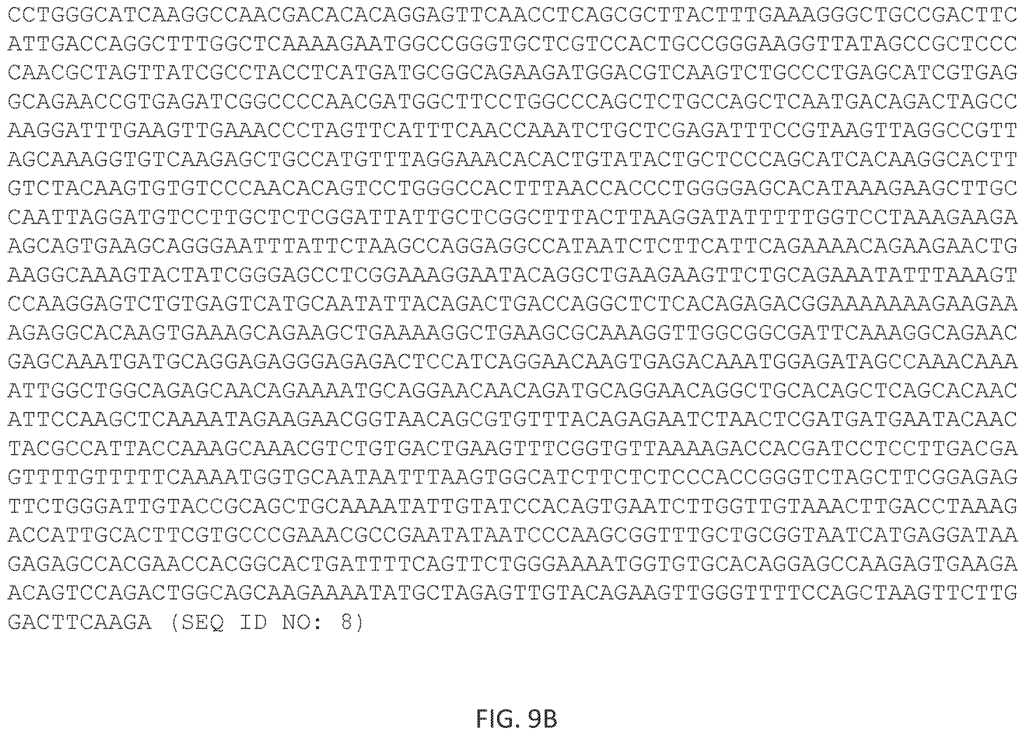

Additionally, venous blood samples were obtained from 6 donors and subsequently analyzed using a prototype GENEXPERT® cartridge at 0, 1, 3, 5 and 7 hours after draw and at 21° C., 25° C., 28° C. and 35° C. FIG. 7 shows the data from this analysis and demonstrates that the TBP score is more stable with time and temperature as compared to the TB score.

### Example 3: Performance Validation of the TBP-Score for Diagnosing Tuberculosis

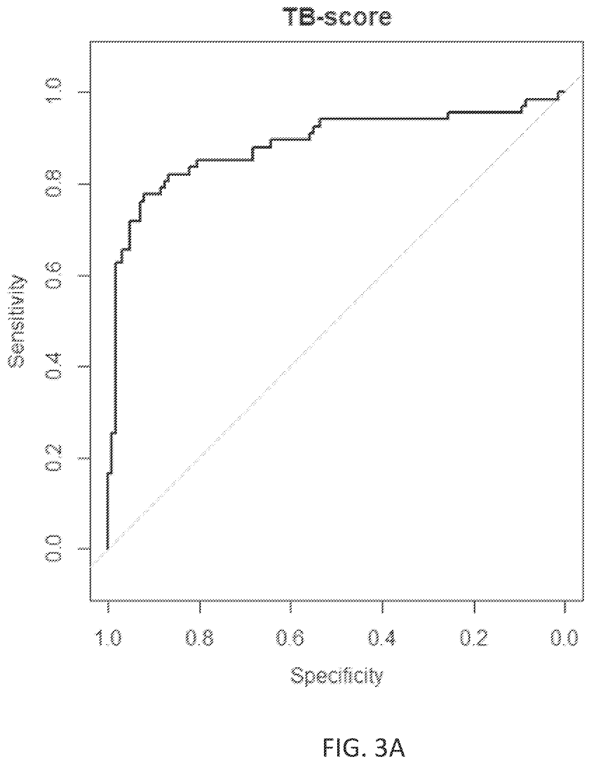

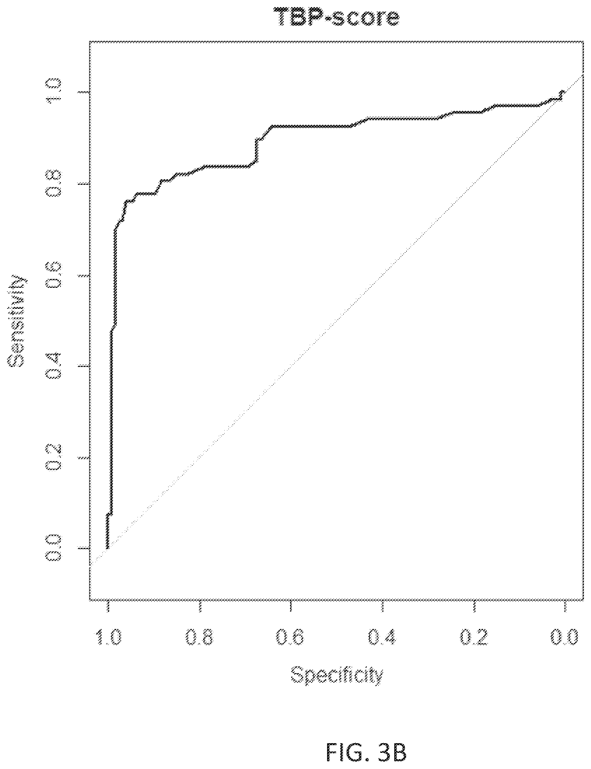

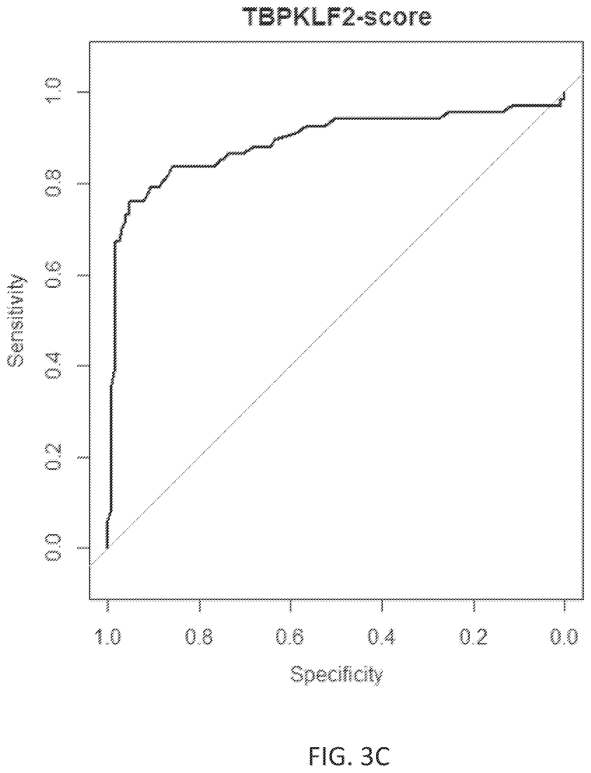

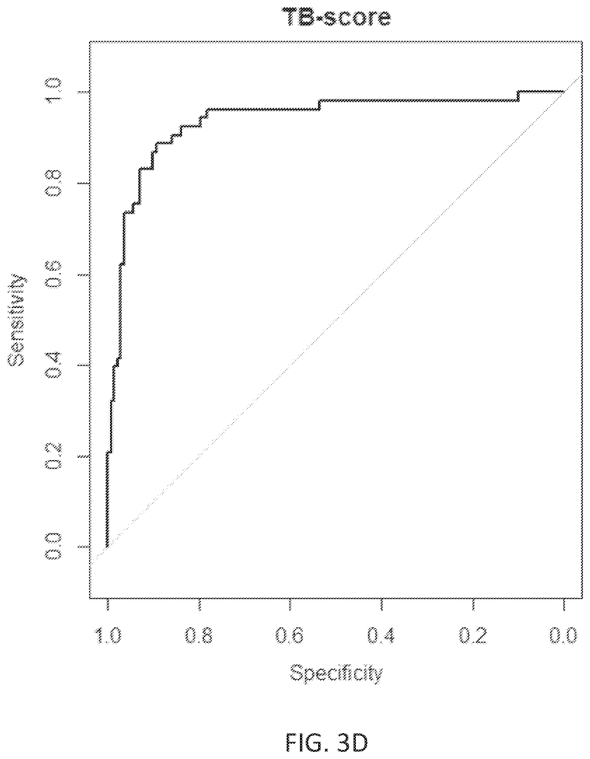

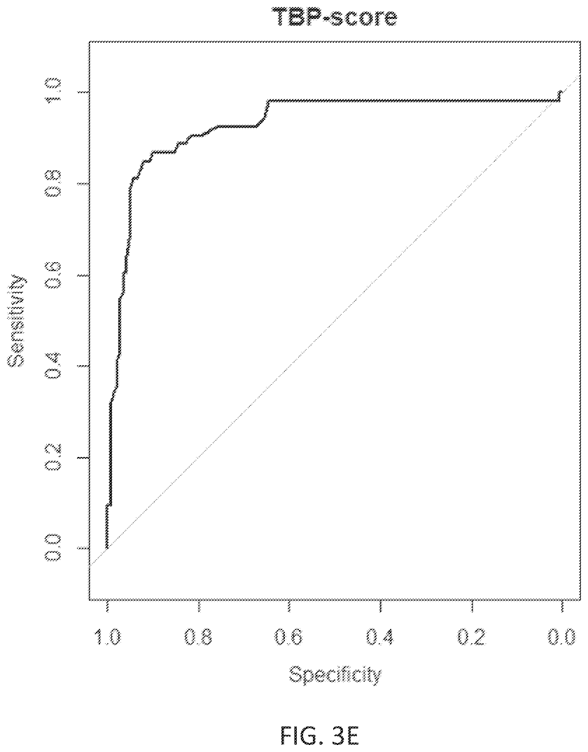

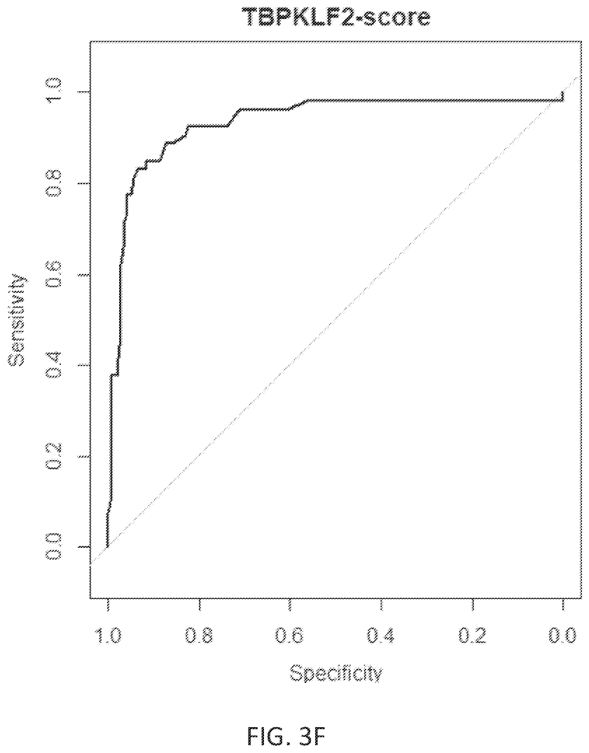

A total of 201 banked whole blood samples in PAXgene buffer from a South African and Peruvian cohort of patients diagnosed with HIV were included in a study by Södersten et al. (Journal of Clinical Microbiology, 2021, 59, 3:1-11). Of the 201 patients, 67 (33.3%) were diagnosed with TB of which 23 (34.3%) were smear negative/culture positive and 44 (65.7%) were smear positive/culture positive. Five patients were defined as “Subclinical TB” and 129 (64.2%) were categorized as “Non-TB” patients (46 LTBI, 83 non LTBI). No patient with possible TB was included. Xpert MTB/RIF on the first sputum sample was able to identify 53 patients (79.1%; smear positive 43/44 [97·7%] and smear negative 10/23 [43·5%]), while Xpert MTB/RIF from all sputum samples detected 57 patients (85%). The PAXgene™ blood samples were analyzed with manually assembled Xpert TB Host Response RUO prototype cartridges, with the same formulation as above. The performance of the Xpert TB Host Response RUO prototype cartridge was evaluated against MTB culture (see FIGS. 3A-C) and against Xpert MTB/RIF (FIGS. 3D-F) using receiver operating characteristic (ROC) analysis for different possible score equations; TB-score=(GBP5+DUSP3)/2-KLF2, TBP-score=(GBP5+DUSP3)/2-TBP or TBPKLF2score=(GBP5+DUSP3)/2−(TBP+KLF2)/2. The resulting AUCs were compared using Bonferroni multiple comparison. No significant difference in AUC (p=1) was observed, suggesting that TBP can fully replace KLF2.

### Example 4: Performance Validation of the TBP-Score for Monitoring Treatment for Active Tuberculosis

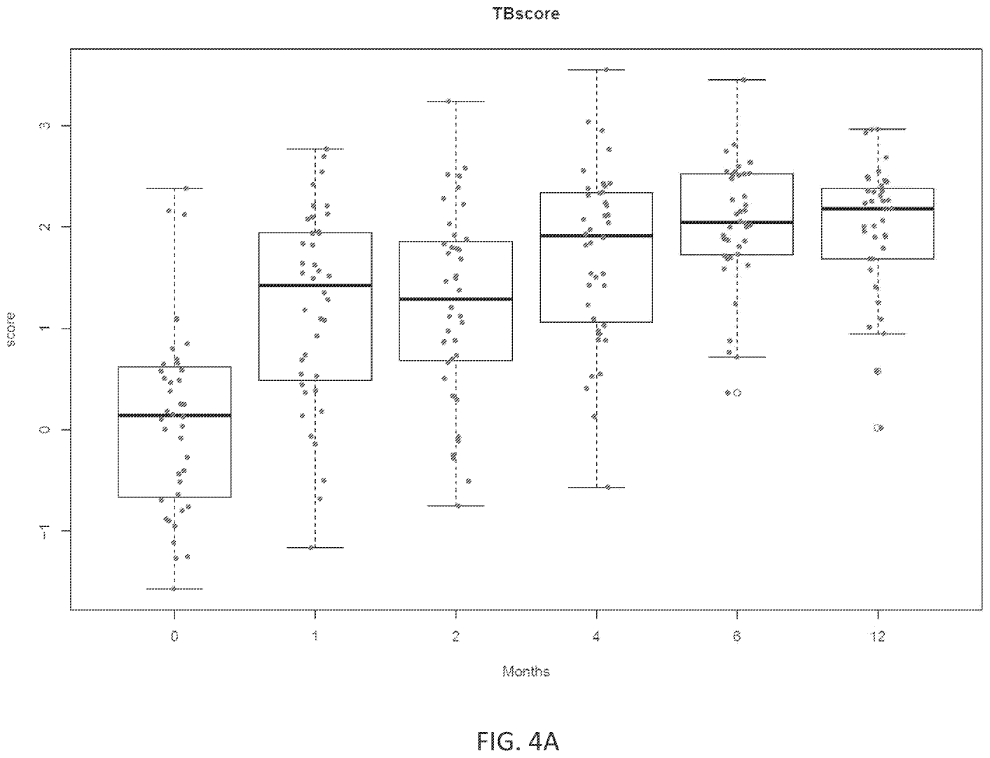

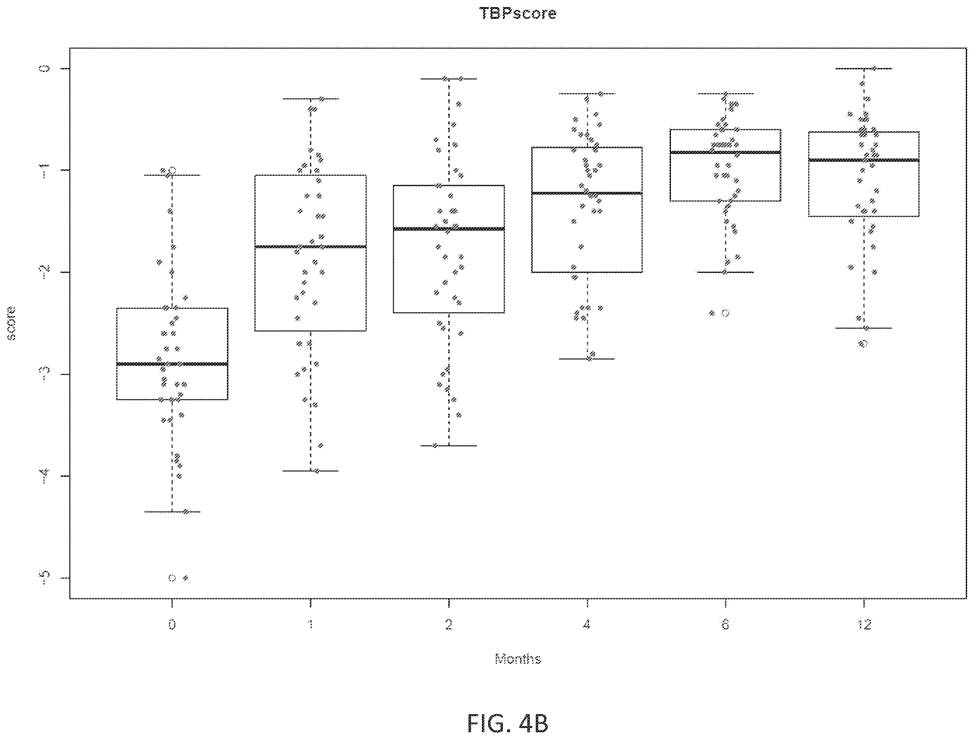

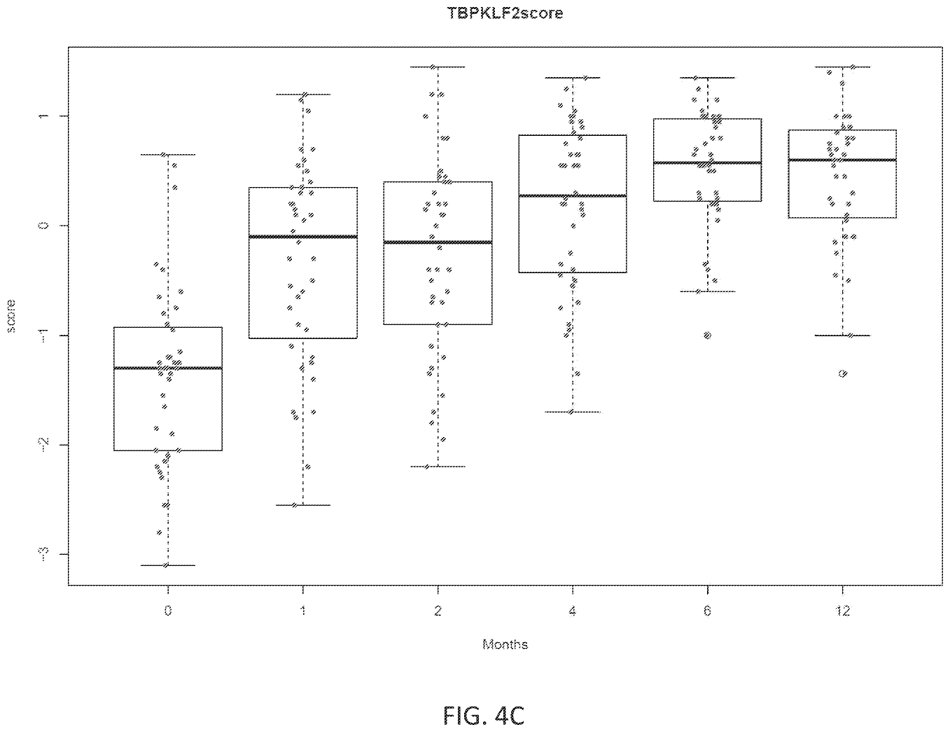

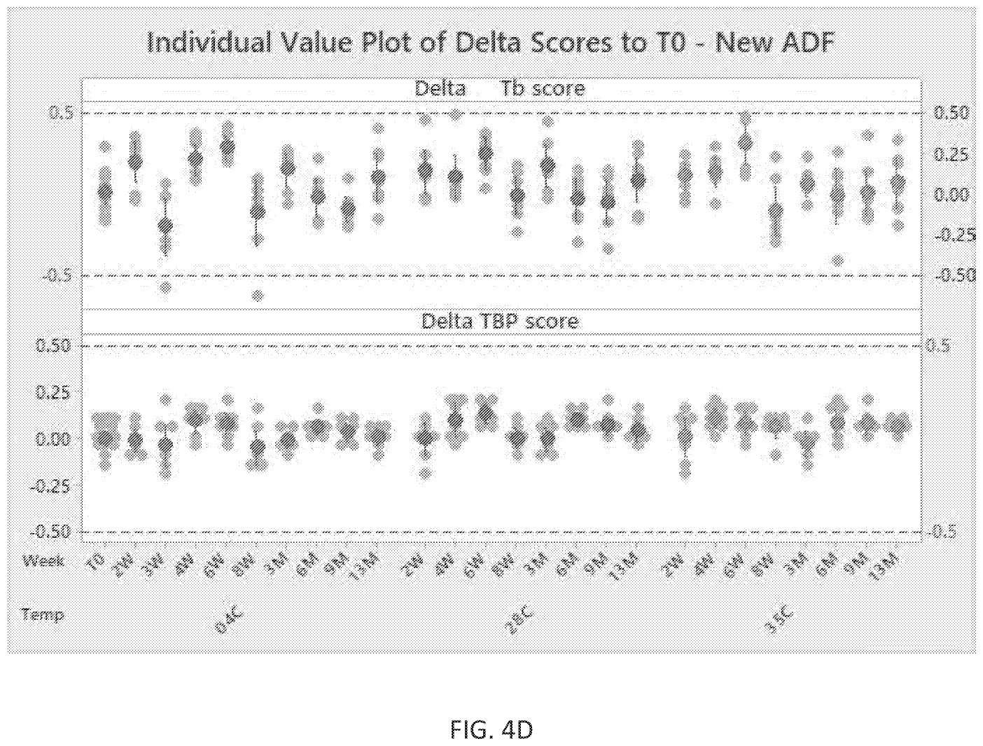

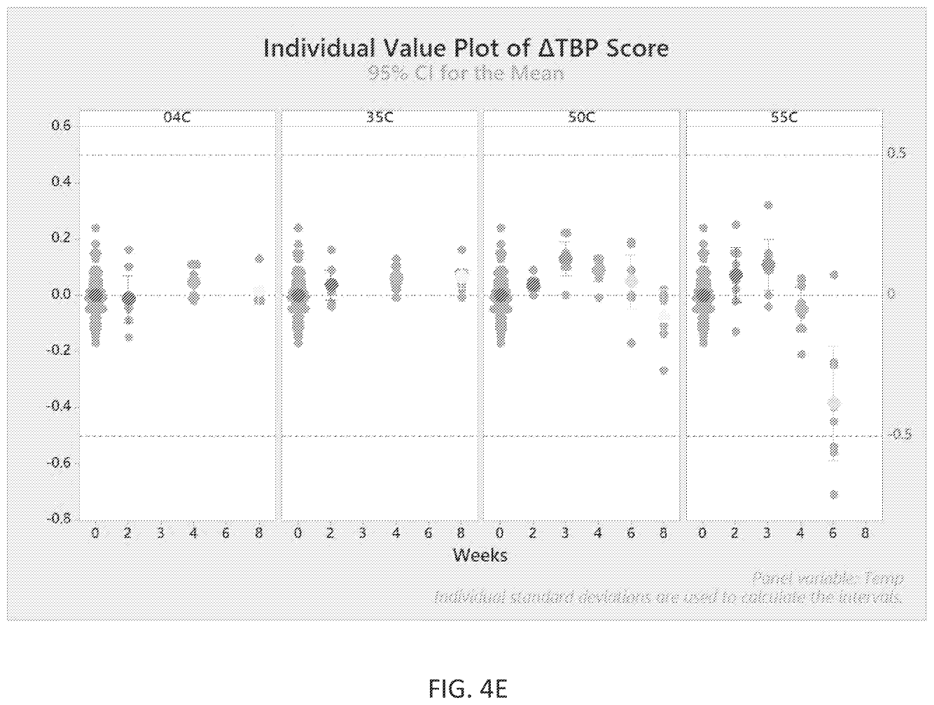

239 PAXgene™ blood samples were obtained from 40 patients prior to initiation of treatment and in follow-up at 1, 2, 4, 6, and 12 months (one 12-month follow-up missing from a cured patient) (now published as Zimmer et al., BMC Res Notes, 2021, 14(1):247). Patients (≥18 years) were enrolled with confirmed TB on MGIT culture. Participants were started on first-line TB treatment after enrollment with 6 (15%) completing treatment by 6 months and the remaining 34 (85%) by 12 months. The samples were analyzed using manually assembled Xpert TB Host Response RUO prototype cartridges. The TB-score (FIG. 4A), TBP-score (FIG. 4B) and TBPKLF2-score (FIG. 4C) changed on a group level with treatment time in a highly similar manner.

### Example 5: Performance Validation of Semi-Quantitative TB Fingerstick Assay

Tuberculosis (TB) is a leading cause of death and places severe pressure on health care services of the developing world. There is insufficient reduction in the number of TB cases to reach the 2020 End TB Strategy milestones, with 3.6 million people with TB still going undiagnosed each year resulting in a 36% gap between reported and estimated active cases. Approximately 30% of patients with TB are not treated and numerous shortfalls have been identified in the investigation and initiation of treatment for TB. The consequent high burden of undiagnosed TB fuels ongoing transmission and delayed treatment worsens treatment outcomes.

Two-thirds of suspected TB cases are seen at public health facilities, however many facilities in high TB prevalence areas still do not have access to efficient TB diagnostic services due to logistical and financial constraints. Currently available diagnostics include radiological and microbiological testing. The expense of and lack of access to test facilities mean that many who require diagnostic testing do not receive it. When testing is available and has been adequately performed, patients are frequently lost to follow-up due to the time lag between testing and availability of results, and overburdened health care systems may fail to respond appropriately to test results. While the highly sensitive and specific GeneXpert® MTB/RIF (GeneXpert®) test with a potential turn-around time of two hours, showed great promise for virtually eliminating this time lag, financial and logistical constraints with inability to fully decentralise testing means effective TB diagnosis remains a challenge. All other available diagnostic modalities have drawbacks for use in primary care facilities. Liquid culture is prone to contamination, takes up to 42 days to deliver a negative result, and is even less accessible than GeneXpert®. Availability of chest X-rays (CXR) in resource-limited settings is currently even lower than for GeneXpert®, is relatively expensive (>10 Euros/CXR), is dependent on skilled personnel for reading and interpretation and although sensitivity is high (98%; 95% CI 95-100%), it has a low specificity (75%, 95% CI 72-79%) (5).

The World Health Organisation (WHO) has recently re-emphasised the need for efficient TB screening. The EnDx: TriageTB Study aims to finalise a point-of-care fingerstick blood test to be used for TB screening. The ultimate goal of the Triage Consortium is to produce a highly sensitive, “rule-out” test for TB diagnosis that is low-cost, easy-to-use, inexpensive and effective for TB screening globally. This test, which has been conceptualised, created and evaluated using serum in African participants in previous EDCTP-funded studies, requires further refinement to ensure global applicability, and requires field-testing using fingerstick blood for operational testing prior to final lock-down for manufacturing. The test is based on measuring individual concentrations of a combination of biomarkers that are present in the blood and have been shown to be associated with active TB.

From previous study experience, only approximately 30% of patients presenting with signs and symptoms consistent with active TB actually test positive for TB. The POC-MBT would identify patients with the highest risk for active TB to be prioritized for further testing, while ruling out the majority of low risk patients with respiratory illnesses other than active TB. Streamlining the diagnostic process would likely significantly reduce overall expenditure in resource-limited settings by reducing unnecessary tests. In addition, healthcare workers would be enabled to focus on testing a smaller number of patients with high risk for active TB. This may increase the rigour with which the diagnostic workup is pursued. Similarly, patients aware of having a high likelihood of TB may show better adherence, improving attendance of return visits for GeneXpert®, culture results or other confirmatory tests to establish a definitive diagnosis and initiate treatment.

Overall, earlier detection and treatment of TB disease should reduce further disease transmission, including that of resistant strains. Thus, in addition to contributing to Sustainable Goal 3.3—towards ending the TB epidemic, the study will contribute to Goal 3.8—towards achieving universal health care coverage and access to quality essential health care services.

Cut-off definition and technical validation of a RT PCR in vitro diagnostic semi quantitative TB Fingerstick test with qualitative cut off values for the detection of a specific human host response in individuals presumptive for active tuberculosis (TB) disease from human capillary or venous EDTA whole blood was assessed. The TB Fingerstick test includes assessment of expression levels of messenger ribonucleic acid (mRNA) from a three gene signature (GBP5, DUSP3, TBP). Cutoffs were empirically determined from proof-of-concept study data from a wide geographical coverage. All data were obtained from POC settings and represented fresh EDTA blood sampling from venous or fingerstick.

A preliminary performance of the assay was conducted using 722 samples (68 Fingerstick samples obtained from North Africa and Middle East hospitalized patients in Sweden; 195 Fingerstick samples obtained from South Africa, Uganda, Gambia, India, and Vietnam candidates patients enrolled in ENDxTB program; and 459 venous samples obtained from South Africa, Uganda, Philippines, India, Gambia, Vietnam candidates in TB clinical trial). The 722 samples were also tested using the MTB/RIF Ultra and the results compared. Results from the MTB/RIF Ultra included 539 negative and 183 positive for TB.

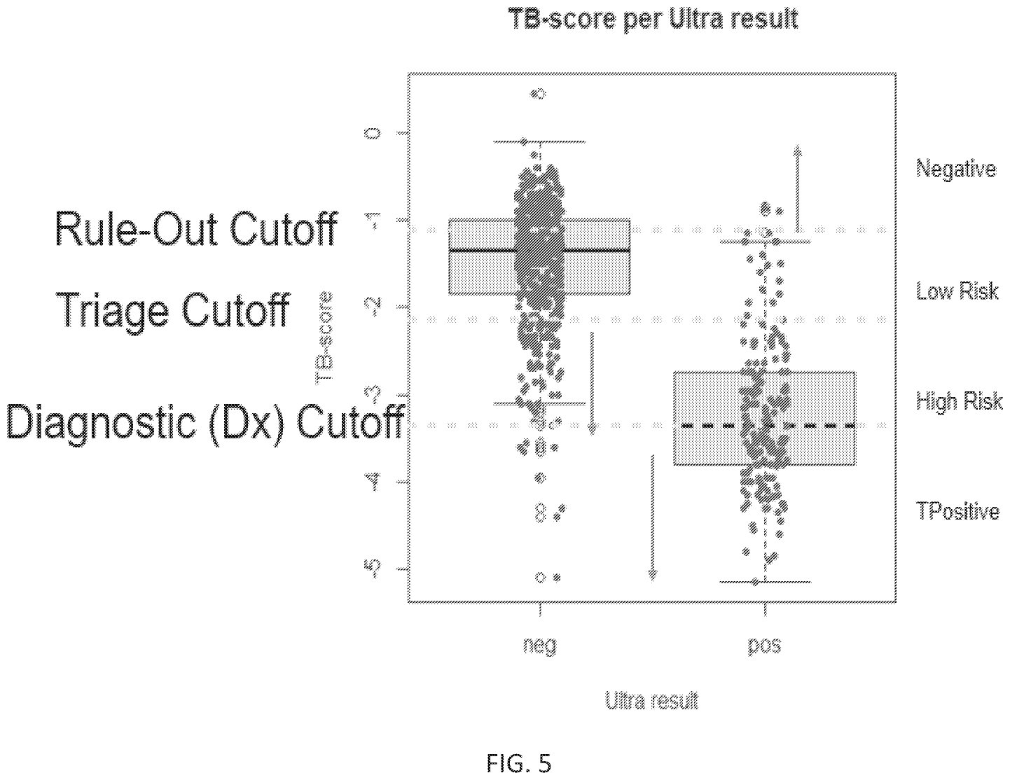

A score was computed based on the obtained expression values which is assessed against three cut-off values in turn separating the patients into 4 risk-based categories: Positive, High Risk, Low Risk and Negative (see FIG. 5). The cut-off values have the following performance:
A diagnostic cut-off with ≥98% specificity and ≥45% sensitivity for active TB.
A triage cut-off with ≥70% specificity and ≥90% sensitivity for active TB and
A rule-out cut-off with ≥99% specificity and ≥30% sensitivity for identifying TB negatives.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that changes can be made without departing from the spirit and scope of the invention(s).

1. A method of identifying if a patient has tuberculosis, a risk of having tuberculosis, or does not have tuberculosis, the method comprising:
a) measuring levels of expression of DUSP3, GBP5, and TBP biomarkers in a biological sample from the patient;
b) identifying the patient as having i) tuberculosis, ii) high risk for having tuberculosis, iii) low risk for having tuberculosis, or iv) no tuberculosis infection based on the expression levels measured in step (a).

2. A method of treating tuberculosis in a patient, the method comprising:
a) measuring levels of expression of DUSP3, GBP5, and TBP biomarkers in a biological sample from the patient;
b) identifying that the patient has tuberculosis or has a high risk for having tuberculosis based on the expression levels measured in step (a); and
c) administering at least one tuberculosis treatment to the patient.

3. The method of claim 2, wherein identifying that the patient has tuberculosis or has a high risk for having tuberculosis based on the expression levels measured in step (a) comprises comparing the level of expression of each biomarker to a respective reference value for that biomarker or to a control to identify that the patient has tuberculosis or has a high risk for having tuberculosis.

4. The method of claim 2, wherein identifying that the patient has tuberculosis or has a high risk for having tuberculosis based on the expression levels measured in step (a) comprises:
b₁) determining a score using the levels of expression measured in step (a);
b₂) identifying that the patient has tuberculosis or has a high risk for having tuberculosis based on the score.

5. The method of claim 4, wherein the score is calculated using the formula:
$$Score = \frac{( GBP 5 + DUSP 3 )}{2} - TBPi$$
wherein GBP5 is the level of expression of the GBP5 biomarker measured in step (a), DUSP3 is the level of expression of the DUSP3 biomarker measured in step (a) and TBP is the level of expression of the TBP biomarker measured in step (a).

6. The method of claim 4, wherein the score is calculated using a formula derived from the analysis of the levels of expression of the DUSP3, GBP5, and TBP biomarkers in a plurality of biological samples obtained from a plurality of subject, preferably wherein:
i) at least one subject in the plurality of subjects has tuberculosis; and
ii) at least one subject in the plurality of subjects does not have tuberculosis, wherein
the at least one subject in the plurality of subjects that has tuberculosis has one of active tuberculosis, subclinical tuberculosis or incipient tuberculosis; and/or
at least one subject in the plurality of subjects that does not have tuberculosis has no tuberculosis infection or has latent tuberculosis.

7. The method of claim 2, wherein when the patient is identified as having tuberculosis or high risk of having tuberculosis, the method further comprises identifying that the patient has or has a high risk of having active tuberculosis, subclinical tuberculosis or incipient tuberculosis based on the expression levels measured in step (a).

8. The method of claim 7, wherein identifying that the patient has active tuberculosis, subclinical tuberculosis, or incipient tuberculosis based on the expression levels measured in step (a) comprises comparing the level of expression of each biomarker to one or more respective reference values for that biomarker or to one or more controls to identify that the patient has active tuberculosis, subclinical tuberculosis, or incipient tuberculosis.

9. The method of claim 7, wherein identifying that the patient has active tuberculosis, subclinical tuberculosis, or incipient tuberculosis based on the expression levels measured in step (a) comprises:
b₁) determining a score using the levels of expression measured in step (a);
b₂) identifying that the patient has active tuberculosis, subclinical tuberculosis, or incipient tuberculosis based on the score,
wherein the score is calculated using a formula derived from the analysis of the levels of expression of the DUSP3, GBP5, and TBP biomarkers in a plurality of biological samples obtained from a plurality of subjects, preferably wherein:
i) at least one subject in the plurality of subjects has active tuberculosis;
ii) at least one subject in the plurality of subjects has subclinical tuberculosis; and
iii) at least one subject in the plurality of subject has incipient tuberculosis.

10. A method of identifying if a patient has active tuberculosis, is at high risk of having active tuberculosis, is at low risk of having active tuberculosis, or does not have tuberculosis, the method comprising:
a) measuring levels of expression of DUSP3, GBP5, and TBP biomarkers in a biological sample from the patient;
b) identifying that the patient:
i) has active tuberculosis;
ii) is at high risk of having active tuberculosis;
iii) is at low risk of having active tuberculosis; or
iv) does not have tuberculosis
based on the expression levels measured in step (a),

11. The method of claim 10, wherein identifying that the patient has active tuberculosis, is at high risk of having active tuberculosis, is at low risk of having active tuberculosis, or does not have tuberculosis based on the expression levels measured in step (a) comprises:
b₁) determining a score using the levels of expression measured in step (a);
b₂) identifying that the patient has tuberculosis, is at high risk of having tuberculosis, is at low risk of having tuberculosis, or does not have tuberculosis based on the score.

12. The method of claim 11, wherein the score is calculated using:
i) the formula:
$$Score = \frac{( GBP 5 + DUSP 3 )}{2} - TBP$$
wherein GBP5 is the level of expression of the GBP5 biomarker measured in step (a), DUSP3 is the level of expression of the DUSP3 biomarker measured in step (a) and TBP is the level of expression of the TBP biomarker measured in step (a); or
ii) a formula derived from the analysis of the levels of expression of the DUSP3, GBP5, and TBP biomarkers in a plurality of biological samples obtained from a plurality of subject, preferably wherein:
i) at least one subject in the plurality of subjects has active or probable tuberculosis; and
ii) at least one subject in the plurality of subjects does not have tuberculosis or has latent tuberculosis.

13. The method of claim 12, wherein identifying that the patient has active tuberculosis, is at high risk of having active tuberculosis, is at low risk of having active tuberculosis, or does not have tuberculosis based on the score comprises:
i) comparing the score to a first, second and third predetermined cutoff value; and
ii) determining that the patient:
a) has active tuberculosis when the score is greater than or equal to the third predetermined (diagnostic) cutoff value;
b) is at high risk of having active tuberculosis when the score is lower than the third predetermined cutoff value and greater than or equal to the second predetermined (triage) cutoff value;
c) is at low risk of having active tuberculosis when the score is less than the second predetermined cutoff value and greater than or equal to the first predetermined (rule-out) cutoff value; or
d) does not have tuberculosis when the score is less than the first predetermined cutoff value.

14. The method of claim 2, further comprising monitoring the response of the patient having tuberculosis to the at least one tuberculosis treatment, the method comprising:
a) measuring levels of expression of DUSP3, GBP5, and TBP biomarkers in a first biological sample from the patient obtained at a first time point;
b) measuring levels of expression of DUSP3, GBP5, and TBP biomarkers in a second biological sample from the patient obtained at a second time point; and
c) determining whether the patient is responding to the at least one tuberculosis treatment by comparing the levels of expression from step (a) and the levels of expression from step (b),
wherein:
(i) the first time point is prior to the administration of the at least one tuberculosis treatment to the patient and the second time point is after the administration of at least one amount of the at least one tuberculosis treatment to the patient; or
(ii) the patient has been administered at least one amount of the at least one tuberculosis treatment prior to the first time point and the second time point is after the administration of at least one additional amount of the at least one tuberculosis treatment.

15. The method of claim 2, wherein the at least one tuberculosis treatment comprises at least one antibiotic, at least one corticosteroid or any combination thereof, preferably wherein the at least one antibiotic is selected from the group consisting of rifampicin, isoniazid, pyrazinamide, ethambutol, rifapentine, ethionamide, moxifloxacin, and streptomycin.

16. The method of claim 2, wherein the biological sample comprises:
i) whole blood, sputum, peripheral blood mononuclear cells, monocytes, or macrophages.
ii) whole blood, whole blood supplemented with anticoagulant agents or whole blood supplemented with RNA-stabilization buffers.

17. The method of claim 2, wherein the biological sample is stored:
i) at a temperature from 4° C. to 35° C. for up to 24 hours prior to measuring the expression levels of the biomarkers;
ii) at room temperature up to 35° C. for up to 24 hours prior to measuring the expression levels of the biomarkers; or
iii) at room temperature up to 35° C. for 0.5 to 8 hours prior to measuring the expression levels of the biomarkers.

18. The method of claim 2, wherein the biomarkers are RNA biomarkers quantified by PCR.

19. A cartridge for identifying if a patient has tuberculosis, a risk of having tuberculosis, or does not have tuberculosis, according to the method of claim 1, the cartridge comprising:
a plurality of processing chambers in fluidic communication, and
a nucleic acid-binding substrate for binding nucleic acid in fluidic communication with the processing chambers,
wherein the processing chambers comprise reagents for lysing cells from a sample, amplification and detection of nucleic acid from the sample, and a composition comprising sets of primers for detecting GBP5, DUSP3, and TBP biomarkers.

20. The cartridge of claim 19, wherein
a) the sets of primers for detecting TBP is selected from:
i) a forward and at least one reverse primer for detecting a sequence of the TBP gene; or
ii) a forward primer comprising a region of at least 15 contiguous nucleotides having a sequence that is at least 85% identical to at least 15 contiguous nucleotides of the TBP gene at exons 3 and/or 4, and at least one reverse primer comprising a region of at least 15 contiguous nucleotides having a sequence that is at least 85% identical to at least 15 contiguous nucleotides of the TBP gene at exons 3 and/or 4, or
iii) a forward primer comprising a region of at least 15 contiguous nucleotides having a sequence that is at least 85% identical to at least 15 contiguous nucleotides of SEQ ID NO: 1 and/or SEQ ID NO: 4, and at least one reverse primer comprising a region of at least 15 contiguous nucleotides having a sequence that is at least 85% identical to at least 15 contiguous nucleotides of SEQ ID NO: 2 and/or SEQ ID NO: 4,
b) the sets of primers for detecting GBP5 is selected from:
i) a forward and at least one reverse primer for detecting a sequence of the GBP5 gene; or
ii) a forward primer comprising a region of at least 15 contiguous nucleotides having a sequence that is at least 85% identical to at least 15 contiguous nucleotides of the GBP5 gene at exons 9 and/or 10, and at least one reverse primer comprising a region of at least 15 contiguous nucleotides having a sequence that is at least 85% identical to at least 15 contiguous nucleotides of the GBP5 gene at exons 9 and/or 10, and
c) the sets of primers for detecting DUSP3 is selected from:
i) a forward and at least one reverse primer for detecting a sequence of the DUSP3 gene; or
ii) a forward primer comprising a region of at least 15 contiguous nucleotides having a sequence that is at least 85% identical to at least 15 contiguous nucleotides of the DUSP3 gene at exons 2 and/or 3, and at least one reverse primer comprising a region of at least 15 contiguous nucleotides having a sequence that is at least 85% identical to at least 15 contiguous nucleotides of the DUSP3 gene at exons 2 and/or 3.
